(19) United States
(12) Patent Application Publication
(10) Pub. No.: US 2023/0131352 A1
(43) Pub. Date: Apr. 27, 2023

(54) REDIRECTION OF TROPISM OF AAV CAPSIDS

(71) Applicant: VOYAGER THERAPEUTICS, INC., Cambridge, MA (US)

(72) Inventors: Mathieu E. NONNENMACHER, Boston, MA (US); Jinzhao HOU, Lexington, MA (US); Wei WANG, Arlington, MA (US); Matthew CHILD, Ashland, MA (US)

(21) Appl. No.: 17/915,393

(22) PCT Filed: Mar. 31, 2021
(86) PCT No.: PCT/US2021/025072

§ 371 Date: Sep. 28, 2022

Related U.S. Application Data
(60) Provisional application No. 63/003,523, filed on Apr. 1, 2020.

(51) Int. Cl. C12N 15/86; C12N 15/10

(52) U.S. Cl. CPC C12N 15/86; C12N 15/1082; C12N 2750/14122; C12N 2750/14143; C12N 2750/14145

(57) ABSTRACT

The disclosure relates to compositions, methods, and processes for the preparation, use, and/or formulation of adeno-associated virus capsid proteins, wherein the capsid proteins comprise targeting peptide inserts for enhanced tropism to a target tissue.

### REFERENCE TO RELEVANT APPLICATIONS

This application claims the benefit of U.S. Provisional Patent Application No. 63/003,523, entitled “REDIRECTION OF TROPISM OF AAV CAPSIDS”, filed Apr. 1, 2020; the contents of which are herein incorporated by reference in their entirety.

### REFERENCE TO THE SEQUENCE LISTING

The present application is being filed along with a Sequence Listing in electronic format. The Sequence Listing is provided as a file entitled 2057_1090PCT_SL.txt, created on Mar. 31, 2021 which is 146,950 bytes in size. The information in the electronic format of the sequence listing is incorporated herein by reference in its entirety.

### FIELD OF THE DISCLOSURE

The disclosure relates to compositions, methods, and processes for the preparation, use, and/or formulation of adeno-associated virus capsid proteins, wherein the capsid proteins comprise targeting peptide inserts for enhanced tropism to a target tissue.

### BACKGROUND

Gene delivery to the adult central nervous system (CNS) remains a major challenge in gene therapy, and engineered AAV capsids with improved brain tropism represent an attractive solution.

Adeno-associated virus (AAV)-derived vectors are promising tools for clinical gene transfer because of their non-pathogenic nature, their low immunogenic profile, low rate of integration into the host genome and long-term transgene expression in non-dividing cells. However, the transduction efficiency of AAV natural variants in certain organs is too low for clinical applications, and capsid neutralization by pre-existing neutralizing antibodies may prevent treatment of a large proportion of patients. For these reasons, major efforts have been devoted to obtaining novel capsid variants with enhanced properties. Of many approaches tested so far, the most significant advances have resulted from directed evolution of AAV capsids using in vitro or in vivo selection of capsid variants created by capsid sequence randomization using either error-prone PCR, shuffling of various parent serotypes or insertion of fully randomized short peptides at defined positions.

In order to perform directed evolution of AAV capsids, the sequence encoding the viral capsid is itself flanked by inverted terminal repeats (ITR) so it can be packaged into its own capsid shell. Following infection of cultured cells or animals by the mixed population of capsids, the DNA encoding capsid variants that have successfully homed into the tissue of interest is recovered by PCR for further rounds of selection. In this approach, all viral DNA species present in a given tissue are recovered, with no discrimination for specific cell types or for vectors able to perform complete transduction (cell surface binding, endocytosis, trafficking, nuclear import, uncoating, second-strand synthesis, transcription). For example, in the case of highly complex tissues containing multiple cell types, such as the central nervous system (CNS), it would be highly preferable to apply a more stringent selective pressure aimed at recovering capsid variants capable of transducing neuron and/or astrocyte rather than microglia or blood vessel endothelial cells.

Attempts at improving the CNS tropism of AAV capsids upon systemic administration have been met with limited success.

Two previous approaches have been used to address this issue. The first strategy used co-infection of cultured cells (Grimm et al., 2008, the contents of which are herein incorporated by reference in its entirety) or in situ animal tissue (Lisowski et al., 2014, the contents of which are herein incorporated by reference in its entirety) with adenovirus, in order to trigger exponential replication of infectious AAV DNA. Another successful approach involved the use of cell-specific CRE transgenic mice (Deverman et al., 2016. the contents of which are herein incorporated by reference in its entirety) allowing viral DNA recombination specifically in astrocytes, followed by recovery of CRE-recombined capsid variants. Both approaches proved successful, allowing the isolation of several capsid variants with enhanced transduction of target cell populations.

This finding suggested that cell type-specific library selection could improve the outcome of directed evolution. However, the transgenic CRE system used by Deverman et al. (2016) is not tractable in other animal species and AAV variants selected by directed evolution in mouse tissue do not show similar properties in large animals. Therefore, it would be necessary to perform the entire directed evolution process directly in non-human primates to increase the probability of translatability in human subjects. None of the previously described transduction-specific approaches are amenable to large animal studies because: 1) many tissues of interest (e.g. CNS) are not readily accessible to adenovirus co-infection, 2) the specific Ad tropism itself would bias the library distribution, and 3) large animals are typically not amenable to transgenesis and cannot be genetically engineered to express CRE recombinase in defined cell types.

To address this problem, we have developed a broadly-applicable functional AAV capsid library screening platform for cell type-specific biopanning in non-transgenic animals. In the TRACER (Tropism Redirection of AAV by Cell type-specific Expression of RNA) platform system, the capsid gene is placed under the control of a cell type-specific promoter to drive capsid mRNA expression in the absence of helper virus co-infection. This RNA-driven screen increases the selective pressure in favor of capsid variants which transduce a specific cell type.

The TRACER platform allows generation of AAV capsid libraries whereby specific recovery and subcloning of capsid mRNA expressed in transduced cells is achieved with no need for transgenic animals or helper virus co-infection. Since mRNA transcription is a hallmark of full transduction, these methods will allow identification of fully infectious AAV capsid mutants. In addition to its higher stringency, this method allows identification of capsids with high tropism for particular cell types using libraries designed to express CAP mRNA under the control of any cell-specific promoter such as, but not limited to, synapsin-1 promoter (neurons), GFAP promoter (astrocytes), TBG promoter (liver), CAMK promoter (skeletal muscle), MYH6 promoter (cardiomyocytes).

### SUMMARY OF THE DISCLOSURE

The present disclosure provides compositions and methods for the engineering and/or redirecting the tropism of AAV capsids. Also provided herein are peptides which may be inserted into AAV capsid sequences to increase the tropism of the capsid for a particular tissue. In one aspect, the peptides may be used to target the capsids to brain or regions of the brain or the spinal cord. In one aspect, the present disclosure provides the adaptation of the rodent TRACER method for use in non-human primate (NHP). In one aspect, the present disclosure provides methods for orthogonal studies for the engineering and/or redirecting the tropism of AAV capsids.

The present disclosure presents methods for generating one or more variant AAV capsid polypeptides. In certain embodiments, the variant AAV capsid polypeptides exhibit at least one of improved transduction or increased cell or tissue specificity, relative to a parental AAV capsid polypeptide. In certain embodiments, the method includes: (a) generating a library of variant AAV capsid polypeptides, wherein said library includes (i) a plurality of capsid polypeptides having a region of randomized sequence of 2, 3, 4, 5, 6, 7, 8, or 9 consecutive amino acids, or (ii) a plurality of capsid polypeptides from more than one parental AAV capsid polypeptide; (b) generating an AAV vector library by cloning the capsid polypeptides of libraries (a)(i) or (a)(ii) into AAV vectors, wherein the AAV vectors include a first promoter and a second promoter, wherein said second promoter drives capsid mRNA expression in the absence of helper virus co-infection.

In certain embodiments, the first promoter is AAV2 P40. In certain embodiments, the second promoter is a ubiquitous promoter. In certain embodiments, the first promoter is AAV2 P40 and the second promoter is a ubiquitous promoter.

In certain embodiments, the first promoter is AAV2 P40. In certain embodiments, the second promoter is a cell-type-specific promoter. In certain embodiments, the first promoter is AAV2 P40 and the second promoter is a cell-type-specific promoter.

In certain embodiments, the promoter is selected from any promoter listed in Table 2. In certain embodiments, the ubiquitous or cell-specific promoter allows the expression of RNA encoding the capsid polypeptides.

In certain embodiments, the method includes recovery of the RNA encoding the capsid polypeptides. In certain embodiments, the method includes determining the sequence of the capsid polypeptides. In certain embodiments, the capsid polypeptides recovered exhibit increased target cell transduction or target cell specificity (tropism) as compared to a parental capsid polypeptide.

In certain embodiments, the target cell is a primate cell. In certain embodiments, the primate cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

In certain embodiments, the AAV vectors comprise a first promoter and a second promoter, wherein the second promoter is located the downstream of the capsid gene and drives its anti-sense RNA expression in the absence of helper virus co-infection.

In certain embodiments, the first promoter is AAV2 P40 and the second promoter is a ubiquitous promoter. In certain embodiments, the first promoter is AAV2 P40 and the second promoter is a cell-specific promoter. In certain embodiments, the ubiquitous or cell-specific promoter allows the expression of gene encoding the capsid polypeptide of variant AAV in an anti-sense direction, resulting in the anti-sense RNA. In certain embodiments, the method included the recovery of the anti-sense RNA that can be converted to RNA encoding the variant AAV capsid polypeptide that is used to determine the sequence of the variant AAV capsid polypeptides.

In certain embodiments, the variant AAV capsid polypeptide exhibits increased target cell transduction or target cell specificity (tropism) as compared to a parental capsid polypeptide.

The present disclosure provides a method for generating a variant AAV capsid polypeptides, wherein the method is conducted in a non-human primate (NHP) and, relative to a parental AAV capsid polypeptide said variant AAV capsid polypeptides exhibit at least one of improved transduction or increased cell or tissue specificity, said method comprising: (a) generating a library of variant AAV capsid polypeptides, wherein said library comprises (i) a plurality of capsid polypeptides having a contiguous region of randomized sequence of 2, 3, 4, 5, 6, 7, 8, or 9 amino acids, or (ii) a plurality of capsid polypeptides having a noncontiguous (e.g., split) region of randomized sequence of 2, 3, 4, 5, 6, 7, 8, or 9 amino acids, or (iii) a plurality of capsid polypeptides from more than one parental AAV capsid polypeptide; (b) generating an AAV vector library by cloning the capsid polypeptides of libraries (i) or (ii) or (iii) into AAV vectors, wherein said AAV vectors comprise a first promoter and a second promoter, wherein said second promoter drives capsid mRNA expression in the absence of helper virus co-infection.

In certain embodiments, the first promoter used for the methods described herein is an AAV2 P40 promoter and the second promoter is a ubiquitous promoter. In certain embodiments, the first promoter used for the methods described herein is an AAV2 P40 promoter and the second promoter is a cell-type specific promoter.

In certain embodiments, each of the promoters used in the methods described herein may be a promoter such as, but not limited to, a B29 promoter, Immunoglobulin heavy chain promoter, CD45 promoter, Mouse INF-β promoter, CD45 SV40/CD45 promoter, WASP promoter, CD43 promoter, CD43 SV40/CD43 promoter, CD68 promoter, GPIIb promoter, CD14 promoter, CD2 promoter, Osteocalcin, Bone sialoprotein, OG-2 promoter, GFAP promoter, Vga, Vglut2, NSE/RU5′ promoter, SYN1 promoter, Neurofilament light chain, VGF, Nestin, Chx10, PrP, Dkk3, Math5, Ptf1a, Pcp2, Nefh, gamma-synuclein gene (SNCG), Grik4, Pdgfra, Chat, Thy1.2, hVmd2, Thy1, Modified αA-crystallin, hRgp, mMo, Opn4, RLBP1, Glast, Foxg1, hVmd2, Trp1, Six3, ex36, Grm6-SV40 eukaryotic promoter, hVmd2, Dct, Rpc65, mRho, Irbp, hRho, Pcp2, Rhodopsin, mSo, MLC2v promoter, αMHC promoter, rat troponin T (Tnnt2), Tie2, Tcf21, ECAD, NKCC2, KSPC, NPHS1, SGLT2, SV40/bAlb promoter, SV40/hAlb promoter, Hepatitis B virus core promoter, Alpha fetoprotein, Surfactant protein B promoter, Surfactant protein C promoter, Desmin, Mb promoter, Myosin, Dystrophin, dMCK and tMCK, Elastase-1 promoter, PDX1 promoter, Insulin promoter, Slco1c1, tie, cadherin, ICAM-2, claudin 1, Cldn5, Flt-1 promoter, Endoglin promoter.

In certain embodiments, the promoter(s) of the method (e.g., ubiquitous or cell-specific) allow for the expression of RNA encoding the capsid polypeptides. In certain embodiments, the method described herein comprises recovering the RNA encoding the capsid polypeptides from a target tissue and determining the sequence of the capsid polypeptides.

In certain embodiments, the recovered capsid polypeptides exhibit increased target cell transduction or target cell specificity (tropism) as compared to a parental capsid polypeptide. In some embodiments, the capsid polypeptide may demonstrate increased target cell transduction or target cell specificity (tropism), wherein the target cell is a primate cell. In some embodiments, the capsid polypeptide may demonstrate increased target cell transduction or target cell specificity (tropism), wherein the target cell is a neuronal cell, a neural stem cell, an astrocyte, an oligodendrocyte, a microglial cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

In some embodiments, the AAV vectors used in the methods described herein comprise a first promoter and a second promoter, wherein the second promoter is located downstream of the capsid gene and drives its anti-sense RNA expression in the absence of helper virus co-infection. In such embodiments, the first promoter may be an AAV2 P40 promoter and the second promoter a ubiquitous promoter. In other embodiments, the first promoter is an AAV2 P40 promoter and the second promoter is a cell-specific promoter.

In some embodiments, the promoter (e.g., ubiquitous or cell-specific) allows the expression of a gene encoding the capsid polypeptide of variant AAV in an anti-sense direction, resulting in the anti-sense RNA. In certain embodiments, the methods described herein comprise recovering the anti-sense RNA that can be converted to RNA encoding the variant AAV capsid polypeptide that is used for determining the sequence of the variant AAV capsid polypeptides. In some embodiments, the resulting variant AAV capsid polypeptide exhibits increased target cell transduction or target cell specificity (tropism) as compared to a parental capsid polypeptide. In some embodiments, the capsid polypeptide may demonstrate increased target cell transduction or target cell specificity (tropism), wherein the target cell is a primate cell. In some embodiments, the capsid polypeptide may demonstrate increased target cell transduction or target cell specificity (tropism), wherein the target cell is a neuronal cell, a neural stem cell, an astrocyte, an oligodendrocyte, a microglial cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

In some embodiments, the methods described herein are conducted in one or more non-human primates (NHP) and simultaneously or subsequently conducted orthogonally in one or more additional species or cell types. In certain embodiments, the one or more additional species is selected from the group consisting of mouse, rat, rabbit, guinea pig, ferret, fish, hamster, bird, pig, sheep, dog, cat, insect, or worm. In certain embodiments, the one or more cell types is a brain microvascular endothelial cell (BMVEC), optionally of human origin.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A method of making a vector or plurality of vectors. e.g., a vector library, comprising:

(a) a first plurality of nucleic acid molecules comprising a first promoter and a second promoter, wherein the second promoter expresses capsid RNA, e.g., mRNA, in the absence of helper virus co-infection; and a second plurality of nucleic acid molecules encoding:
(i) a plurality of variant AAV capsid polypeptides having a region of randomized sequence of at least 2, 3, 4, 5, 6, 7, 8, or 9 consecutive amino acids, or
(ii) a plurality of variant AAV capsid polypeptides from more than one parental AAV capsid polypeptide; and

incubating the first plurality of nucleic acids and second plurality of nucleic acids under conditions suitable to generate the vector or plurality of vectors, e.g., vector library:

thereby making the vector or plurality of vectors, e.g., vector library.

2. The method of embodiment 1, wherein the first plurality of nucleic acid molecules is introduced into a host cell, e.g., a prokaryotic cell, optionally wherein the first plurality of nucleic acid molecules is introduced prior to, concurrently with, or subsequently to the second plurality of nucleic acid molecules.

3. The method of embodiment 1 or 2, wherein the second plurality of nucleic acid molecules encodes a plurality of variant AAV capsid polypeptides having a region of randomized sequence of at least 5, 6, 7, 8, or 9 consecutive amino acids.

4. The method of embodiment 1 or 2, wherein the second plurality of nucleic acid molecules encodes a plurality of variant AAV capsid polypeptides from more than one parental AAV capsid polypeptide.

5. The method of any one of the preceding embodiments, wherein the method further comprises (b) generating an AAV particle comprising the vector or plurality of vectors.

6. The method of any one of the preceding embodiments, wherein the method further comprises (c) administering the AAV particle or plurality of AAV particles to a non-human subject (e.g., a non-human primate (NHP), mouse, and/or rat), or a eukaryotic cell (e.g., a HEK293 cell, a human brain microvascular endothelial cell (hBMVEC), and/or an NHP BMVEC).

7. A method of making an AAV particle or plurality of AAV particles, comprising:

(a) providing a host cell comprising a vector or a plurality of vectors, e.g., a vector library, comprising nucleic acid molecules encoding:
(i) a plurality of variant AAV capsid polypeptides having a region of randomized sequence of at least 2, 3, 4, 5, 6, 7, 8, or 9 consecutive amino acids, or
(ii) a plurality of variant AAV capsid polypeptides from more than one parental AAV capsid polypeptide;

wherein the vector or a plurality of vectors comprise a first promoter and a second promoter, wherein the second promoter expresses capsid RNA, e.g., mRNA, in the absence of helper virus co-infection; and

(b) incubating the host cell under conditions suitable to enclose the vector or plurality of vectors in a capsid polypeptide; thereby making the AAV particle or plurality of AAV particles.

8. The method of embodiment 7, wherein the host cell is an insect cell (e.g., a Sf9 cell) or a mammalian cell (e.g., a HEK293 cell).

9. The method of embodiment 7 or 8, wherein the method further comprises (c) administering the AAV particle or plurality of AAV particles to a non-human subject (e.g., an NHP, mouse, and/or rat), or a eukaryotic cell (e.g., a HEK293 cell, an hBMVEC, and/or an NHP BMVEC).

10. A method of making a variant AAV capsid polypeptide, comprising:

(a) providing a plurality of vectors, e.g., a vector library, comprising nucleic acid molecules encoding:
(i) a plurality of variant AAV capsid polypeptides having a region of randomized sequence of at least 2, 3, 4, 5, 6, 7, 8, or 9 consecutive amino acids, or
(ii) a plurality of variant AAV capsid polypeptides from more than one parental AAV capsid polypeptide;

wherein the vectors comprise a first promoter and a second promoter, wherein the second promoter expresses capsid RNA, e.g., mRNA, in the absence of helper virus co-infection; and

(b) generating a plurality of AAV particles comprising the plurality of vectors, e.g., the vector library, of (a);

thereby making the variant AAV capsid polypeptide.

11. The method of embodiment 9, wherein the method further comprises (c) administering the AAV particle or plurality of AAV particles to a non-human subject (e.g., an NHP, a mouse, and/or a rat), or a eukaryotic cell (e.g., a HEK293 cell, an hBMVEC cell, and/or a NHP BMVEC cell)

12. A method of making a variant AAV capsid polypeptide, comprising:

(a) providing a plurality of vectors, e.g., a vector library, comprising nucleic acid molecules encoding:
(i) a plurality of variant AAV capsid polypeptides having a region of randomized sequence of at least 2, 3, 4, 5, 6, 7, 8, or 9 consecutive amino acids, or
(ii) a plurality of variant AAV capsid polypeptides from more than one parental AAV capsid polypeptide;

wherein the vectors comprise a first promoter and a second promoter, wherein the second promoter expresses capsid RNA, e.g., mRNA, in the absence of helper virus co-infection;

(b) generating a plurality of AAV particles comprising the plurality of vectors, e.g., the vector library, of (a); and

(c) administering the plurality of AAV particles to an NHP.

13. The method of any one of embodiments 5-12, wherein the step (b) of generating the AAV particle or the plurality of AAV particles, comprises: (i) providing a host cell comprising the vector library; and (ii) incubating the host cell under conditions suitable to enclose the vectors in a capsid polypeptide, optionally wherein the host cell is an insect cell (e.g., a Sf9 cell) or a mammalian cell (e.g., a HEK293 cell).

14. A method of making a variant AAV capsid polypeptide, comprising administering an AAV particle or plurality of AAV particles to an NHP, which comprise a plurality of vectors. e.g., a vector library, comprising nucleic acid molecules encoding:

(i) a plurality of variant AAV capsid polypeptides having a region of randomized sequence of at least 2, 3, 4, 5, 6, 7, 8, or 9 consecutive amino acids, or

(ii) a plurality of variant AAV capsid polypeptides from more than one parental AAV capsid polypeptide;

wherein the vectors comprise a first promoter and a second promoter, wherein the second promoter expresses capsid RNA, e.g., mRNA, in the absence of helper virus co-infection.

15. The method of any one of embodiments 7-14, wherein the plurality of vectors, e.g., vector library, comprises nucleic acid molecules encoding a plurality of variant AAV capsid polypeptides having a region of randomized sequence of at least 5, 6, 7, 8, or 9 consecutive amino acids.

16. The method of any one of the preceding embodiments, wherein the region of randomized sequence comprises a peptide insert of at least 4, 5, 6, 7, 8, or 9 consecutive amino acids.

17. The method of embodiment 16, wherein the insert is present in a surface-exposed hypervariable loop chosen from loop I, loop IV, loop VI, and/or loop VIII.

18. The method of embodiment 16 or 17, wherein the insert is present in loop IV and/or loop VIII of the parental AAV capsid polypeptide, optionally wherein the parental AAV capsid polypeptide comprises an AAV5 capsid polypeptide or an AAV9 capsid polypeptide.

19. The method of any one of embodiments 16-18, wherein the insert is present immediately subsequent to a position selected from 454-461 of the parental sequence.

20. The method of any one of embodiments 16-19, wherein the insert is present immediately subsequent to a position selected from 586-588 of the parental sequence.

21. The method of any one of embodiments 7-14, wherein the plurality of vectors. e.g., vector library, comprises nucleic acid molecules encoding a plurality of variant AAV capsid polypeptides from more than one parental AAV capsid polypeptide.

22. The method of any one of the preceding embodiments, wherein the parental AAV capsid polypeptide comprises an AAV5 capsid polypeptide or an AAV9 capsid polypeptide, e.g., an AAV9 capsid polypeptide of SEQ ID NO: 2.

23. The method of any one of embodiments 6, 9, or 11-22, wherein the AAV particle or plurality of particles is administered via intravenous administration, intraventricular administration, or intra-cisternal magna (ICM) injection.

24. The method of any one of the preceding embodiments, wherein the second promoter is located 3′ relative to a transgene encoding the variant AAV capsid polypeptide and results in anti-sense RNA expression of the variant AAV capsid in the absence of helper virus co-infection.

25. The method of any one of the preceding embodiments, wherein the method further comprises (d) collection and/or isolation of a target cell or tissue from the non-human subject (e.g., a NHP, mouse, and/or rat), the eukaryotic cell (e.g., a HEK293 cell, an hBMVEC cell, and/or a NHP BMVEC cell), or the NHP.

26. The method of embodiment 25, wherein the target cell or tissue is collected and/or isolated at least about 5 to 21 days, e.g., about 5-10 days, 5-14 days, 7-10 days, 7-14 days, 7-21 days, 10-14 days, 10-21 days, 14-17 days, 5 days, 7 days, 10 days, 14 days, or 21 days, following administration of the AAV particles.

27. The method of embodiment 25 or 26, wherein the target cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

28. The method of any one of embodiments 25-27, wherein the target tissue is:
(i) a CNS tissue, a PNS tissue, and/or a peripheral tissue; and/or
(ii) a brain tissue (e.g., a cortex (e.g., frontal, parietal, occipital, temporal), thalamus, hypothalamus, striatum, putamen, caudate nucleus, hippocampus, entorhinal cortex, basal ganglia, deep cerebellar nuclei), a spinal cord tissue, a dorsal root ganglion, a muscle tissue, a liver tissue, a heart tissue, a gastrocnemius muscle tissue, a soleus muscle tissue, a pancreas tissue, a kidney tissue, a spleen tissue, a lung tissue, an adrenal glands tissue, a stomach tissue, a sciatic nerve tissue, a saphenous nerve tissue, a thyroid gland tissue, an eye tissue (with or without optic nerve), a pituitary gland tissue, a skeletal muscle tissue (rectus femoris), a colon tissue, a duodenum tissue, an ileum tissue, a jejunum tissue, a skin tissue of the leg, a superior cervical ganglia tissue, a urinary bladder tissue, an ovary tissue, a uterus tissue, a prostate gland tissue, and/or a testes tissue.

29. The method of any one of the preceding embodiments, wherein the method further comprises (e) recovery of RNA and/or antisense RNA encoding the variant AAV capsid polypeptides from the target cell or tissue, e.g., as described in Examples 15-17.

30. The method of embodiment 29, wherein the RNA encoding the variant AAV capsid polypeptides is enriched and/or reverse transcribed to cDNA, optionally wherein the cDNA is amplified, e.g., by PCR, e.g., as described in Examples 15-17.

31. The method of any one of the preceding embodiments, wherein the method further comprises (f) determination of the sequence of the variant AAV capsid polypeptides, e.g., by next generation sequence (NGS), e.g., as described in Examples 15-17.

32. The method of any one of the preceding embodiments, wherein the method further comprises (g) evaluating, e.g., measuring, the amount of the variant AAV capsid polypeptides (e.g., the amount of DNA encoding the variant AAV capsid polypeptides, the amount of RNA encoding the variant AAV capsid polypeptides, or the amount of the variant AAV capsid polypeptides), e.g., by NGS, e.g., as described in Examples 15-17, in a target cell, or tissue.

33. The method of embodiment 32, wherein the amount of the variant AAV capsid polypeptide in the target cell or tissue is increased relative to a reference level, wherein the reference level comprises the amount of a wild-type AAV capsid polypeptide (e.g., a wild-type AAV9 or AAV5 capsid polypeptide), or a parental capsid polypeptide (e.g., a parental capsid polypeptide comprising SEQ ID NO: 2) in the target cell or tissue.

34. The method of embodiment 32 or 33, wherein an increase in the amount of the variant AAV capsid polypeptide in the target cell or tissue is indicative of or predictive of an increased level of transduction of the target cell or tissue, relative to a wild-type AAV capsid polypeptide (e.g., a wild-type AAV9 or AAV5 capsid polypeptide), or parental capsid polypeptide (e.g., a parental capsid polypeptide comprising SEQ ID NO: 2).

35. The method of any one of embodiments 32-34, wherein an increase in the amount of the variant AAV capsid polypeptide in the target cell or tissue is indicative of or predictive of increased tropism for the target cell or tissue, relative to a wild-type AAV capsid polypeptide (e.g., a wild-type AAV9 or AAV5 capsid polypeptide), or parental capsid polypeptide (e.g., a parental capsid polypeptide comprising SEQ ID NO: 2).

36. The method of any one of the preceding embodiments, which comprises repeating one, two, three, four, five, six, or all of steps (a)-(g), at least 1-5 times, e.g., at least 1-3 times, 2-3 times, 2-4 times, 3-5 times, 4-5 times, 1 time, 2 times, or 3 times.

37. The method of any one of the preceding embodiments, wherein responsive to an increase in one, two, or all of:

(i) the amount of the variant AAV capsid polypeptide in the target cell or tissue;

(ii) the level of transduction of the variant AAV capsid polypeptide in the target cell or tissue;

(iii) the tropism of the variant AAV capsid polypeptide for the target cell or tissue

as compared to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2),

(f) selecting the variant AAV capsid polypeptide, e.g., for use in an AAV particle for delivering a payload to the target cell or tissue, e.g., of a subject, e.g. a human subject.

38. The method of any one of embodiments 29-37, wherein the target cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

39. The method of any one of embodiments 29-38, wherein the target tissue is:
(i) a CNS tissue, a PNS tissue, and/or a peripheral tissue; and/or
(ii) a brain tissue (e.g., a cortex (e.g., frontal, parietal, occipital, temporal), thalamus, hypothalamus, striatum, putamen, caudate nucleus, hippocampus, entorhinal cortex, basal ganglia, deep cerebellar nuclei), a spinal cord tissue, a dorsal root ganglion, a muscle tissue, a liver tissue, a heart tissue, a gastrocnemius muscle tissue, a soleus muscle tissue, a pancreas tissue, a kidney tissue, a spleen tissue, a lung tissue, an adrenal glands tissue, a stomach tissue, a sciatic nerve tissue, a saphenous nerve tissue, a thyroid gland tissue, an eye tissue (with or without optic nerve), a pituitary gland tissue, a skeletal muscle tissue (rectus femoris), a colon tissue, a duodenum tissue, an ileum tissue, a jejunum tissue, a skin tissue of the leg, a superior cervical ganglia tissue, a urinary bladder tissue, an ovary tissue, a uterus tissue, a prostate gland tissue, and/or a testes tissue.

40. A variant AAV capsid polypeptide or plurality of variant AAV capsid polypeptides made by the method of any one of the preceding embodiments.

41. The variant AAV capsid polypeptide or plurality of variant AAV capsid polypeptides of embodiment 40, which transduces a target cell or tissue at an increased level relative to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2).

42. The variant AAV capsid polypeptide or plurality of variant AAV capsid polypeptides of embodiment 40 or 41, which has increased tropism for a target cell or tissue relative to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2).

43. The variant AAV capsid polypeptide or plurality of variant AAV capsid polypeptides of any one of embodiments 40-42, wherein the target cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

44. The variant AAV capsid polypeptide or plurality of variant AAV capsid polypeptides of any one of embodiments 40-43, wherein the target tissue is:
(i) a CNS tissue, a PNS tissue, and/or a peripheral tissue; and/or
(ii) a brain tissue (e.g., a cortex (e.g., frontal, parietal, occipital, temporal), thalamus, hypothalamus, striatum, putamen, caudate nucleus, hippocampus, entorhinal cortex, basal ganglia, deep cerebellar nuclei), a spinal cord tissue, a dorsal root ganglion, a muscle tissue, a liver tissue, a heart tissue, a gastrocnemius muscle tissue, a soleus muscle tissue, a pancreas tissue, a kidney tissue, a spleen tissue, a lung tissue, an adrenal glands tissue, a stomach tissue, a sciatic nerve tissue, a saphenous nerve tissue, a thyroid gland tissue, an eye tissue (with or without optic nerve), a pituitary gland tissue, a skeletal muscle tissue (rectus femoris), a colon tissue, a duodenum tissue, an ileum tissue, a jejunum tissue, a skin tissue of the leg, a superior cervical ganglia tissue, a urinary bladder tissue, an ovary tissue, a uterus tissue, a prostate gland tissue, and/or a testes tissue.

45. The method or variant AAV capsid polypeptide or plurality of variant AAV capsid polypeptides of any one of the preceding embodiments, wherein the variant AAV capsid polypeptide further comprises:
(i) an amino acid substitution at position K449, e.g., a K449R substitution, numbered according to SEQ ID NO: 2;
(ii) the amino acid sequence of SEQ ID NO: 2, or an amino acid sequence at least 90% (e.g., at least 92, 95, 96, 97, 98, or 99%) identical thereto;
(iii) an amino acid sequence encoded by the nucleotide sequence of SEQ ID NO: 1, or a nucleotide at least 90% (e.g., at least 92, 95, 96, 97, 98, or 99%) identical thereto; and/or
(iv) the amino acid sequence of SEQ ID NO: 3, or an amino acid sequence at least 90% (e.g., at least 92, 95, 96, 97, 98, or 99%) identical thereto, optionally provided that position 449 is not K. e.g., is R.

46. A vector or plurality of vectors, e.g., a vector library, made by the method of embodiment 1 or 2.

47. A vector or plurality of vectors. e.g., a vector library, comprising nucleic acid molecules encoding:

(i) a plurality of variant AAV capsid polypeptides having a region of randomized sequence of at least 2, 3, 4, 5, 6, 7, 8, or 9 consecutive amino acids, or

(ii) a plurality of variant AAV capsid polypeptides from more than one parental AAV capsid polypeptide;

wherein the vector or a plurality of vectors comprise a first promoter and a second promoter, wherein said second promoter expresses capsid RNA, e.g., mRNA, in the absence of helper virus co-infection.

48. The method of any one of embodiments 1-39 or 45, or the vector or plurality of vectors of embodiment 46 or 47, wherein:

(i) the first and/or second promoter is located 5′ relative to a transgene encoding the variant AAV capsid polypeptide;

(ii) the first and/or second promoter is located 3′ relative to a transgene encoding the variant AAV capsid polypeptide;

(iii) the first promoter is located 5′ relative to a transgene encoding the variant AAV capsid polypeptide and the second promoter is located 3′ relative to the transgene encoding the variant AAV capsid polypeptide; or

(iv) the first promoter is located 3′ relative to a transgene encoding the variant AAV capsid polypeptide and the second promoter is located Y relative to the transgene encoding the variant AAV capsid polypeptide.

49. The method of any one of embodiments 1-39, 45, or 48, or the vector or plurality of vectors of any one of embodiments 46-48, wherein the first promoter is AAV2 P40.

50. The method of any one of embodiments 1-39, 45, or 4849, or the vector or plurality of vectors of any one of embodiments 46-49, wherein the second promoter is a ubiquitous promoter, a cell-type-specific promoter, or a tissue-specific promoter, or a functional variant thereof.

51. The method of embodiment 50, wherein the cell-type specific promoter or tissue-specific promoter is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof.

52. The method of any one of embodiments 1-39, 45, or 48-51, or the vector or plurality of vectors of any one of embodiments 46-51, wherein the second promoter is selected from any of those listed in Table 2, or a functional variant thereof.

53. The method of any one of embodiments 1-39, 45, or 48-52, or the vector or plurality of vectors of any one of embodiments 46-52, wherein the second promoter is a neuron specific promoter or an astrocyte-specific promoter, optionally wherein:
(i) the neuron specific promoter is a synapsin promoter; and/or
(ii) the astrocyte-specific promoter is a GFAP promoter.

54. The method of any one of embodiments 1-39, 45, or 48-53, or the vector or plurality of vectors of any one of embodiments 46-53, wherein the second promoter is chosen from a human elongation factor 1α-subunit (EF1α) promoter; cytomegalovirus (CMV) immediate-early enhancer and/or promoter; a chicken β-actin (CBA)) and/or its derivative CAG promoter; a β glucuronidase (GUSB) promoter; a ubiquitin C (UBC) promoter; or a functional variant thereof.

55. The method of any one of embodiments 1-39, 45, or 48-54, or the vector or plurality of vectors of any one of embodiments 46-54, wherein the first promoter is AAV2 P40 and the second promoter is a ubiquitous promoter, e.g., a human elongation factor 1α-subunit (EF1α) promoter; cytomegalovirus (CMV) immediate-early enhancer and/or promoter; a chicken β-actin (CBA)) and/or its derivative CAG promoter; a β glucuronidase (GUSB) promoter; a ubiquitin C (UBC) promoter; or a functional variant thereof.

56. The method of any one of embodiments 1-39, 45, or 48-55, or the vector or plurality of vectors of any one of embodiments 46-55, wherein the first promoter is AAV2 P40 and the second promoter is a cell-type-specific promoter, e.g., a neuron-specific promoter or an astrocyte-specific promoter, or functional variant thereof.

57. The method of any one of embodiments 1-39, 45, or 48-56, or the vector or plurality of vectors of any one of embodiments 46-56, wherein:
(i) the first promoter is AAV2 P40 and the second promoter is a neuron-specific promoter;
(ii) the first promoter is AAV2 P40 and the second promoter is a synapsin promoter;
(iii) the first promoter is AAV2 P40 and the second promoter is an astrocyte-specific promoter; and/or
(iv) the first promoter is AAV2 P40 and the second promoter is a GFAP promoter.

58. The method of any one of embodiments 1-39, 45, or 48-57, or the vector or plurality of vectors of any one of embodiments 46-57, wherein the use of a ubiquitous or a cell-specific promoter results in the expression of RNA encoding the capsid polypeptides.

59. The method of any one of embodiments 1-39, 45, or 48-58, or the vector or plurality of vectors of any one of embodiments 46-58, wherein the use of a ubiquitous or a cell-specific promoter results in the expression of a gene encoding the variant AAV capsid polypeptide in an anti-sense direction, resulting in the anti-sense RNA.

60. The method of any one of embodiments 1-39, 45, or 48-59, or the vector or plurality of vectors of any one of embodiments 46-59, wherein the first and/or second promoters is operably linked to a transgene encoding the variant AAV capsid polypeptide.

61. The method of any one of embodiments 1-39, 45, or 48-60, or the vector or plurality of vectors of any one of embodiments 46-60, wherein the vector or plurality of vectors further comprise an inverted terminal repeat (ITR) sequence.

62. The method or the vector or plurality of vectors of embodiment 61, wherein the ITR sequence is positioned 5′ relative to the encoded variant AAV capsid polypeptide.

63. The method or the vector or plurality of vectors of embodiment 61 or 62, wherein the ITR sequence is positioned 3′ relative to the encoded variant AAV capsid polypeptide.

64. The method of any one of embodiments 1-39, 45, or 48-63, or the vector or plurality of vectors of any one of embodiments 46-63, wherein the vector or plurality of vectors comprises an ITR sequence positioned 5′ relative to the encoded variant AAV capsid polypeptide, and an ITR sequence positioned 3′ relative to the encoded variant AAV capsid polypeptide.

65. The method of any one of embodiments 1-39, 45, or 48-64, or the vector or plurality of vectors of any one of embodiments 46-64, wherein the vector or plurality of vectors further comprises a poly A signal sequence.

66. The method of any one of embodiments 1-39, 45, or 48-65, or the vector or plurality of vectors of any one of embodiments 46-65, wherein the vector or plurality of vectors comprise in 5′ to 3′ order:

(i) a 5′ adeno-associated (AAV) ITR;

(ii) a ubiquitous promoter or a tissue specific promoter, optionally wherein:
(a) the ubiquitous promoter or the tissue specific promoter is selected from any of those listed in Table 2, or a functional variant thereof;
(b) the ubiquitous promoter is a human elongation factor 1α-subunit (EF1α) promoter; cytomegalovirus (CMV) immediate-early enhancer and/or promoter; a chicken β-actin (CBA)) and/or its derivative CAG promoter; a β glucuronidase (GUSB) promoter; a ubiquitin C (UBC) promoter; or a functional variant thereof;
(c) the tissue-specific promoter is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof;
(d) the tissue-specific promoter is a neuron-specific promoter, optionally a snyapsin promoter; and/or
(e) the tissue-specific promoter is an astrocyte-specific promoter, optionally a GFAP promoter;

(iii) an AAV2 P40 promoter

(iv) a transgene encoding the variant AAV capsid polypeptide;

(v) a poly A signal sequence; and

(vi) a 3′ AAV ITR.

67. The method of any one of embodiments 1-39, 45, or 48-65, or the vector or plurality of vectors of any one of embodiments 46-65, wherein the vector or plurality of vectors comprise in 5′ to 3′ order:

(i) a 5′ adeno-associated (AAV) ITR;

(ii) an AAV2 P40 promoter;

(iii) a ubiquitous promoter or a tissue specific promoter, optionally wherein:
(a) the ubiquitous promoter or the tissue specific promoter is selected from any of those listed in Table 2, or a functional variant thereof,
(b) the ubiquitous promoter is a human elongation factor 1α-subunit (EF1α) promoter; cytomegalovirus (CMV) immediate-early enhancer and/or promoter; a chicken pi-actin (CBA)) and/or its derivative CAG promoter; a D glucuronidase (GUSB) promoter; a ubiquitin C (UBC) promoter; or a functional variant thereof;
(c) the tissue-specific promoter is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof;
(d) the tissue-specific promoter is a neuron-specific promoter, optionally a snyapsin promoter; and/or
(e) the tissue-specific promoter is an astrocyte-specific promoter, optionally a GFAP promoter;

(iv) a transgene encoding the variant AAV capsid polypeptide;

(v) a poly A signal sequence; and

(vi) a 3′ AAV ITR.

68. A library comprising a plurality of variant AAV capsid polypeptides generated according to the method of any one of embodiments 10-39, 45, or 48-67.

69. An AAV particle or plurality of AAV particles made by the method of embodiment 7 or 8.

70. A cell comprising the variant AAV capsid polypeptide or plurality of variant AAV capsid polypeptides of any one of embodiments 40-45, the vector or plurality of vectors, e.g., vector library of any one of embodiments 46-65, the library of variant AAV capsid polypeptides of embodiment 68, or the AAV particle or plurality of AAV particles of embodiment 69.

71. The cell of embodiment 70, which is an insect cell (e.g., an Sf9 cell), a prokaryotic cell, or a eukaryotic cell (e.g., a mammalian cell, a human cell, an NHP cell, an HEK293 cell, an hBMVEC, and/or an NHP BMVEC).

72. The cell of embodiment 70, which is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

73. A method for generating a variant AAV capsid polypeptides, wherein relative to a parental AAV capsid polypeptide said variant AAV capsid polypeptides exhibit at least one of improved transduction or increased cell or tissue specificity, said method comprising:

(a) generating a library of variant AAV capsid polypeptides, wherein said library comprises
(i) a plurality of capsid polypeptides having a region of randomized sequence of 2, 3, 4, 5, 6, 7, 8, or 9 consecutive amino acids, or
(ii) a plurality of capsid polypeptides from more than one parental AAV capsid polypeptide;

(b) generating an AAV vector library by cloning the capsid polypeptides of libraries (i) or (ii) into AAV vectors, wherein said AAV vectors comprise a first promoter and a second promoter, wherein said second promoter drives capsid mRNA expression in the absence of helper virus co-infection.

74. The method of embodiment 73, wherein the first promoter is AAV2 P40 and the second promoter is a ubiquitous promoter.

75. The method of embodiment 73, wherein the first promoter is AAV2 P40 and the second promoter is a cell-type-specific promoter.

76. The method of embodiment 74 or embodiment 75, wherein the promoter is selected from any of those listed in Table 2.

77. The method of embodiment 76, wherein the ubiquitous or cell-specific promoter allows the expression of RNA encoding the capsid polypeptides.

78. The method of embodiment 77, further comprising the recovery of said RNA encoding the capsid polypeptides and determining the sequence of said capsid polypeptides.

79. The method of embodiment 78, wherein the capsid polypeptides recovered exhibit increased target cell transduction or target cell specificity (tropism) as compared to a parental capsid polypeptide.

80. The method of embodiment 79, wherein the target cell is a primate cell.

81. The method of embodiment 80, wherein the primate cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

82. The method of embodiment 73, wherein said AAV vectors comprise a first promoter and a second promoter, wherein said second promoter is located downstream of the capsid gene and drives its anti-sense RNA expression in the absence of helper virus co-infection.

83. The method of embodiment 82, wherein the first promoter is AAV2 P40 and the second promoter is a ubiquitous promoter.

84. The method of embodiment 82, wherein the first promoter is AAV2 P40 and the second promoter is a cell-specific promoter.

85. The method of embodiment 83 or embodiment 84, wherein the ubiquitous or cell-specific promoter allows the expression of a gene encoding the capsid polypeptide of variant AAV in an anti-sense direction, resulting in the anti-sense RNA.

86. The method of embodiment 85, wherein said method further comprises the recovery of said anti-sense RNA that can be converted to RNA encoding said variant AAV capsid polypeptide that is used for determining the sequence of said variant AAV capsid polypeptides.

87. The method of embodiment 86, wherein said variant AAV capsid polypeptide exhibits increased target cell transduction or target cell specificity (tropism) as compared to a parental capsid polypeptide.

88 The method of embodiment 87, wherein the target cell is a primate cell.

89. The method of embodiment 88, wherein the target cell is a neuronal cell, a neural stem cell, an astrocyte, a oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.

### BRIEF DESCRIPTION OF THE DRAWINGS

The foregoing and other objects, features, and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1A and FIG. 1B are maps of wild-type AAV capsid gene transcription and CMV-CAP vectors. FIG. 1A shows transcription of VP1, VP2 and VP3 AAV transcripts from wildtype AAV genome. Transcription start sites of each viral promoter are indicated. SD, splice donor, SA, splice acceptor. Sequence of start codons for each reading frame is indicated. Translation of AAP and VP3 is performed by leaky scanning of the major mRNA.

FIG. 1B shows the structure of the CMV-p40 dual promoter vectors used to determine the minimal regulatory sequences necessary for efficient virus production. The pREP2ΔCAP vector shown at the bottom is obtained by deletion of most CAP reading frame and is used to provide the REP protein in trans.

FIG. 2 shows the design of improved pREP helper vectors. The MscI fragment deletion removes the C-terminal part of VP proteins, which is necessary for capsid formation. Asterisks represent early stop codons introduced to disrupt the coding potential of VP1, VP2 and VP3 reading frames.

FIG. 3 shows the design of Pro9 vectors for the in vivo analysis of the second-generation vectors. Architecture of all three vectors is based on the BstEII construct. AAV9 capsid RNA is placed under control of P40 and CMV, hSyn1 or GFAP promoters, respectively.

FIG. 4 shows the design of intronic Pro9 vectors harboring a hybrid CMV/Globin intron for the in vitro analysis of intronic second generation vectors. AAV9 capsid RNA is placed under control of P40 and CBA, hSyn1 or GFAP promoters in a tandem (forward) configuration (top) or in an inverted configuration (bottom). In the inverted promoter vectors, an extra SV40 polyadenylation site is added at the 3′ extremity to allow polyadenylation of antisense CAP9 transcripts.

FIG. 5A, FIG. 5B, and FIG. 5C provide in vitro evidence that the presence of the P40 promoter downstream of Synapsin or Gfabc1D promoters does not relieve the repression of either promoter in HEK-293T cells.

FIG. 6 illustrates the basic tenets of the TRACER platform.

FIG. 7 illustrates features of the TRACER platform including the use of a tissue specific promoter and RNA recovery.

FIG. 8 provides some embodiments of the TRACER production architecture.

FIG. 9A and FIG. 9B provide diagrams representing capsid gene transcription of natural AAV (FIG. 9A) and TRACER libraries (FIG. 9B).

FIG. 10 provides a comparison between traditional vDNA recovery and 2^{nd} generation vRNA recovery.

FIG. 11 provides an overview of the use of cell-specific RNA expression for targeted evolution.

FIG. 12 is a diagram of the AAV6. AAV5 and AAV-DJ capsid peptide display libraries used for in vivo evolution (SEQ ID NOS 27-32, respectively, in order of appearance).

FIG. 13 is a diagram of the Gibson assembly library cloning procedure.

FIG. 14 is a diagram of the AAV9 capsid peptide display libraries used for in vivo evolution (SEQ ID NOS 33-42, respectively, in order of appearance).

FIG. 15A and FIG. 15B present the method used for library construction. FIG. 15A shows the sequence of the insertion site used to introduce random libraries (SEQ ID NOS 43-46, respectively, in order of appearance). FIG. 15B provides a description of the assembly procedure.

FIG. 16 provides an exemplary diagram of cloning-free rolling circle procedure used for library amplification (SEQ ID NO 47; NNK₇).

FIG. 17 provides a diagram comparing the traditional and cloning-free methods.

FIG. 18 provides a diagram of protelomerase monomer processing (SEQ ID NOS 59-61, respectively, in order of appearance).

FIG. 19 provides an exemplary diagram of cloning-free DNA amplification by rolling circle amplification.

FIG. 20 provides the sequence of the codon-mutant AAV9 library shuttle designed to minimize wild-type contamination (SEQ ID NOS 33-34 and 48-52, respectively, in order of appearance).

FIG. 21 provides a description of AAV9 peptide libraries biopanning.

FIG. 22 illustrates the recovery process from an initial pool with recovery at 50%.

FIG. 23 provides the results of the astrocyte synthetic library NGS analysis (SEQ ID NOS 53-58, 53-58, and 53-58, respectively, in order of appearance).

FIG. 24 illustrates some embodiments of a multi-species (e.g., rodent) study followed by next generation sequencing (NGS).

FIG. 25A, FIG. 25B and FIG. 25C provide diagrams of external barcoding for NGS analysis and recovery of full-length capsid variants. A general barcode pair is shown (FIG. 25A). Full ITR-to-ITR constructs are shown with the barcode pair 5′ of the CAP sequence (FIG. 25B) and 3′ of the CAP sequence (FIG. 25C).

FIG. 26 provides virus production and RNA splicing with several configurations of intronic barcoded platforms. A general ITR-to-ITR construct with intronic barcode examples is shown (SEQ ID NO: 62-66).

FIG. 27 provides peptide display capsid library configurations. FIG. 27 discloses left “6mer full scan” sequences as SEQ ID NOS 69-76. “3-position scan” sequences as SEQ ID NOS 77-79, “7mer full scan” sequences as SEQ ID NOS 80-82, 78, 79, and 83, and right “6mer full scan” sequences as SEQ ID NOS 84-89, respectively, in order of appearance.

FIG. 28A and FIG. 28B provide diagrams of identification and design of non-human primate (NHP) TRACER AAV capsid libraries with de novo synthesis of lead candidates (FIG. 28A) and pooling of capsid libraries (FIG. 28B).

FIG. 29 provides a diagram of identification and design of orthogonal evolution TRACER AAV capsid libraries.

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

According to the present disclosure, AAV particles with enhanced tropism for a target tissue (e.g., CNS) are provided, as well as associated processes for their targeting, preparation, formulation and use. Targeting peptides and nucleic acid sequences encoding the targeting peptides are provided. These targeting peptides may be inserted into an AAV capsid protein sequence to alter tropism to a particular cell-type, tissue, organ or organism, n vivo, ex vivo or in vitro.

As used herein, an “AAV particle” or “AAV vector” comprises a capsid protein and a viral genome, wherein the viral genome comprises at least one payload region and at least one inverted terminal repeat (ITR). The AAV particle and/or its component capsid and viral genome may be engineered to alter tropism to a particular cell-type, tissue, organ or organism.

As used herein, “viral genome” or “vector genome” refers to the nucleic acid sequence(s) encapsulated in an AAV particle. A viral genome comprises a nucleic acid sequence with at least one payload region encoding a payload and at least one ITR.

As used herein, a “payload region” is any nucleic acid molecule which encodes one or more “payloads” of the disclosure. As non-limiting examples, a payload region may be a nucleic acid sequence encoding a payload comprising an RNAi agent or a polypeptide.

As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism.

The AAV particles and payloads of the disclosure may be delivered to one or more target cells, tissues, organs, or organisms. In a preferred embodiment, the AAV particles of the disclosure demonstrate enhanced tropism for a target cell type, tissue or organ. As a non-limiting example, the AAV particle may have enhanced tropism for cells and tissues of the central or peripheral nervous systems (CNS and PNS, respectively). The AAV particles of the disclosure may, in addition, or alternatively, have decreased tropism for an undesired target cell-type, tissue or organ.

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

AAV vectors of the present disclosure may be produced recombinantly and may be based on adeno-associated virus (AAV) parent or reference sequences. As used herein, a ‘vector’ is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell.

In some embodiments, the AAV particle of the present disclosure is an scAAV.

In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

In some embodiments, the AAV particles of the disclosure comprising a capsid with an inserted targeting peptide and a viral genome, may have enhanced tropism for a cell-type or tissue of the human CNS.

### AAV Capsids

AAV particles of the present disclosure may comprise or be derived from any natural or recombinant AAV serotype. AAV serotypes may differ in characteristics such as, but not limited to, packaging, tropism, transduction and immunogenic profiles. While not wishing to be bound by theory, the AAV capsid protein is often considered to be the driver of AAV particle tropism to a particular tissue.

In some embodiments, an AAV particle may have a capsid protein and ITR sequences derived from the same parent serotype (e.g., AAV2 capsid and AAV2 ITRs). In another embodiment, the AAV particle may be a pseudo-typed AAV particle, wherein the capsid protein and ITR sequences are derived from different parent serotypes (e.g., AAV9 capsid and AAV2 ITRs; AAV2/9).

The AAV particles of the present disclosure may comprise an AAV capsid protein with a targeting peptide inserted into the parent sequence. The parent capsid or serotype may comprise or be derived from any natural or recombinant AAV serotype. As used herein, a “parent” sequence is a nucleotide or amino acid sequence into which a targeting sequence is inserted (i.e., nucleotide insertion into nucleic acid sequence or amino acid sequence insertion into amino acid sequence).

In preferred embodiments, the parent AAV capsid nucleotide sequence is as set forth in SEQ ID NO: 1.

In some embodiments, the parent AAV capsid nucleotide sequence is a K449R variant of SEQ ID NO: 1, wherein the codon encoding a lysine (e.g., AAA or AAG) at position 449 in the amino acid sequence (nucleotides 1345-1347) is exchanged for one encoding an arginine (CGT, CGC, CGA, CGG, AGA, AGG). The K449R variant has the same function as wild-type AAV9.

In some embodiments, the parent AAV capsid amino acid sequence is as set forth in SEQ ID NO: 2.

In some embodiments, the parent AAV capsid amino acid sequence is as set forth in SEQ ID NO: 3.

In some embodiments the parent AAV capsid sequence is any of those shown in Table 1.

TABLE 1
AAV Capsid Sequences

| | SEQ ID | |
|---|---|---|
| Serotype | NO | Reference Information |
| AAV9/hu.14 (nt) | 1 | U.S. Pat. No. 7,906,111 SEQ ID NO: 3; WO2015038958 SEQ ID NO: 11 |
| AAV9/hu.14 (aa) | 2 | U.S. Pat. No. 7,906,111 SEQ ID NO: 123; WO2015038958 SEQ ID NO: 2 |
| AAV9/hu.14 K449R (aa) | 3 | WO2017100671 SEQ ID NO: 45 |

Each of the patents, applications and or publications listed in Table 1 are hereby incorporated by reference in their entirety.

The parent AAV serotype and associated capsid sequence may be any of those known in the art. Non-limiting examples of such AAV serotypes include, AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN. AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a. AAV3b, AAV3-3, AAV4, AAV4-4, AAV6, AAV6.1. AAV6.2, AAV6.1.2, AAV7, AAV7.2, AAV8, AAV9.11. AAV9.13, AAV9.16, AAV9.24, AAV9.45, AAV9.47, AAV9.61, AAV9.68, AAV9.84, AAV9.9, AAV10, AAV I1, AAV12, AAV16.3, AAV24.1, AAV27.3, AAV42.12, AAV42-1b, AAV42-2, AAV42-3a, AAV42-3b, AAV42-4. AAV42-5a, AAV42-5b. AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1. AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61. AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19. AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrb.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21. AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1. AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVth8R A586R mutant, AAVrh8R R533A mutant, AAAV, BAAV, caprine AAV, bovine AAV, AAVhE1.1. AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23. AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5. AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2. AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4. AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5. AAVF6/HSC6, AAVF7/HSC7, AAVF8/HSC8, and/or AAVF9/HSC9 and variants thereof.

In some embodiments, the serotype may be AAVDJ or a variant thereof, such as AAVDJ8 (or AAV-DJ8), as described by Grimm et al. (Journal of Virology 82(12): 5887-5911 (2008), US Publication US20140359799 and U.S. Pat. No. 7,588,772, each of which is herein incorporated by reference in its entirety). The amino acid sequence of AAVDJ8 may comprise two or more mutations in order to remove the heparin binding domain (HBD). As a non-limiting example, the AAV-DJ sequence is as described by SEQ ID NO: 1 in U.S. Pat. No. 7,588,772, the contents of which are herein incorporated by reference in their entirety, and the AAVDJ8 sequence may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, the AAVDJ8 sequence may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the parent AAV capsid sequence comprises an AAV9 sequence.

In some embodiments, the parent AAV capsid sequence comprises an K449R AAV9 sequence.

In some embodiments, the parent AAV capsid sequence comprises an AAVDJ sequence.

In some embodiments, the parent AAV capsid sequence comprises an AAVDJ8 sequence.

In some embodiments, the parent AAV capsid sequence comprises an AAVrh10 sequence.

In some embodiments, the parent AAV capsid sequence comprises an AAV1 sequence.

In some embodiments, the parent AAV capsid sequence comprises AAVF7/HSC7 (SEQ ID NO: 8 and 27 of WO2016049230).

In some embodiments, the parent AAV capsid sequence comprises AAVF15/HSC15 (SEQ ID NO: 16 and 33 of WO2016049230).

In some embodiments, the parent AAV capsid sequence comprises AAVF17/HSC17 (SEQ ID NO: 13 and 35 of WO2016049230).

In some embodiments, the parent AAV capsid sequence comprises an AAV5 sequence. As a non-limiting example, the AAV5 sequence is SEQ ID NO: 4 of U.S. Pat. No. 6,984,517, the contents of which are herein incorporated by reference in their entirety.

While not wishing to be bound by theory, it is understood that a parent AAV capsid sequence comprises a VP1 region. In some embodiments, a parent AAV capsid sequence comprises a VP1, VP2 and/or VP3 region, or any combination thereof. A parent VP1 sequence may be considered synonymous with a parent AAV capsid sequence.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the parent AAV capsid sequence may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, the parent AAV capsid sequence may be encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above.

In some embodiments, the parent sequence is not an AAV capsid sequence and is instead a different vector (e.g., lentivirus, plasmid, etc.). In another embodiment, the parent sequence is a delivery vehicle (e.g., a nanoparticle) and the targeting peptide is attached thereto.

### Targeting Peptides

Disclosed herein are targeting peptides and associated AAV particles comprising a capsid protein with one or more targeting peptide inserts, for enhanced or improved transduction of a target tissue (e.g., cells of the CNS or PNS).

In some embodiments, the targeting peptide may direct an AAV particle to a cell or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei.

In some embodiments, the targeting peptide may direct an AAV particle to a cell or tissue of the PNS. The cell or tissue of the PNS may be, but is not limited to, a dorsal root ganglion (DRG).

The targeting peptide may direct an AAV particle to the CNS (e.g., the cortex) after intravenous administration.

The targeting peptide may direct an AAV particle to the PNS (e.g., DRG) after intravenous administration.

A targeting peptide may vary in length. In some embodiments, the targeting peptide is 3-20 amino acids in length. As non-limiting examples, the targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

A targeting peptide may be contiguous (or continuous) or noncontiguous (or not continuous), or split, or divided across two or more amino acid sequences by intervening amino acid sequences that may vary in length. The contiguous targeting peptide may vary in length. As non-limiting examples, the contiguous targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length. The noncontiguous, or split, targeting peptide may vary in length. As non-limiting examples, the noncontiguous, or split, targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length. The intervening amino acid sequence may vary in length. As non-limiting examples, the intervening targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

Targeting peptides of the present disclosure may be identified and/or designed by any method known in the art. As a non-limiting example, the CREATE system as described in Deverman et al., (Nature Biotechnology 34(2):204-209 (2016)), Chan et al., (Nature Neuroscience 20(8):1172-1179 (2017)), and in International Patent Application Publication Nos. WO2015038958 and WO2017100671, the contents of each of which are herein incorporated by reference in their entirety, may be used as a means of identifying targeting peptides, in either mice or other research animals, such as, but not limited to, non-human primates. As a non-limiting example, the iTransduce system designed in Hanlon et al., 2019 (Hanlon et al., Mol Ther Methods Clin Dev. 2019 Oct. 23; 15:320-332, the contents of which are herein incorporated by reference in its entirety) may be used as a means of identifying targeting peptides, in either mice or other research animals, such as, but not limited to, non-human primates.

Targeting peptides of the present disclosure may be identified and/or designed by any sliding window algorithm known in the art.

Targeting peptides and associated AAV particles may be identified from libraries of AAV capsids comprised of targeting peptide variants. In some embodiments, the targeting peptides may be 5 amino acid sequences (5-mers). In some embodiments, the targeting peptides may be 6 amino acid sequences (6-mers). In some embodiments, the targeting peptides may be 7 amino acid sequences (7-mers). In some embodiments, the targeting peptides may be 9 amino acid sequences (9-mers). The targeting peptides may also differ in their method of creation or design, with non-limiting examples including, random peptide selection, site saturation mutagenesis, and/or optimization of a particular region of the peptide (e.g., flanking regions or central core).

In some embodiments, a targeting peptide library comprises targeting peptides of 7 amino acids (7-mer) in length randomly generated by PCR.

In some embodiments, a targeting peptide library comprises targeting peptides with 3 mutated amino acids. In some embodiments, these 3 mutated amino acids are consecutive, or contiguous, amino acids. In another embodiment, these 3 mutated amino acids are not consecutive, or noncontiguous, or split, amino acids. In some embodiments, the targeting peptide is a 5-mer. In some embodiments, the targeting peptide is a 6-mer. In some embodiments, the parent targeting peptide is a 7-mer. In another embodiment, the parent peptide is a 9-mer.

In some embodiments, a targeting peptide library comprises 7-mer targeting peptides, wherein the amino acids of the targeting peptide and/or the flanking sequences are evolved through site saturation mutagenesis of 3 consecutive amino acids. In some embodiments, NNK (N=any base; K=G or T) codons are used to generate the site saturated mutation sequences.

AAV particles comprising capsid proteins with targeting peptide inserts are generated and viral genomes encoding a reporter (e.g., GFP) encapsulated within. These AAV particles (or AAV capsid library) are then administered to a transgenic rodent (e.g. mouse) by intravenous delivery to the tail vein. Administration of these capsid libraries to cre-expressing mice results in expression of the reporter payload in the target tissue, due to the expression of Cre.

In some embodiments, AAV capsid mRNA expression may be under the control of, or driven by, a cell-type specific promoter. Such capsids, which may compromise targeting peptide inserts and viral genomes encoding a reporter encapsulated within, may be administered, e.g., by intravenous delivery to the tail vein, to a non-transgenic rodent (e.g. mouse), such as but not limited to a C57BL/6 mouse, a BALB/C mouse and a rat. Administration of such capsid libraries to a non-transgenic rodent may result in the expression of the reporter payload in the target tissue, due to the cell-type specific promoter.

In some embodiments, AAV particles comprising capsid proteins with targeting peptide inserts may hereinafter also be referred to as peptide display capsid libraries.

AAV particles and/or viral genomes may be recovered from the target tissue for identification of targeting peptides and associated AAV particles that are enriched, indicating enhanced transduction of target tissue. Standard methods in the art, such as, but not limited to next generation sequencing (NGS), viral genome quantification, biochemical assays, immunohistochemistry and/or imaging of target tissue samples may be used to determine enrichment.

A target tissue may be any cell, tissue or organ of a subject. As non-limiting examples, samples may be collected from brain, spinal cord, dorsal root ganglia and associated roots, liver, heart, gastrocnemius muscle, soleus muscle, pancreas, kidney, spleen, lung, adrenal glands, stomach, sciatic nerve, saphenous nerve, thyroid gland, eyes (with or without optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, or being of interest.

### Targeting Peptide Sequences

In some embodiments, the AAV particle of the disclosure may comprise an AAV capsid polynucleotide with a targeting nucleic acid insert, wherein the targeting nucleic acid insert has a nucleotide sequence substantially comprising any of those as described in Hanlon et al., 2019 (Hanlon et al., Mol Ther Methods Clin Dev. 2019 Oct. 23; 15:320-332, the contents of which are herein incorporated by reference in its entirety). As a non-limiting example, the targeting nucleic acid insert has a nucleotide sequence substantially comprising AAV-S. As a non-limiting example, the targeting nucleic acid insert has a nucleotide sequence substantially comprising AAV-F.

The AAV particle of the disclosure comprising a targeting nucleic acid insert, may have a polynucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

The AAV particle of the disclosure comprising a targeting peptide insert, may have an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

### Use of Targeting Peptides in AAV Particles

Targeting peptides may be stand-alone peptides or may be inserted into or conjugated to a parent sequence. In some embodiments, the targeting peptides are inserted into the capsid protein of an AAV particle.

One or more targeting peptides may be inserted into a parent AAV capsid sequence to generate the AAV particles of the disclosure.

Targeting peptides may be inserted into a parent AAV capsid sequence in any location that results in fully functional AAV particles. The targeting peptide may be inserted in VP1, VP2 and/or VP3. Numbering of the amino acid residues differs across AAV serotypes, and so the exact amino acid position of the targeting peptide insertion may not be critical. As used herein, amino acid positions of the parent AAV capsid sequence are described using AAV9 (SEQ ID NO: 2) as reference.

In some embodiments, the targeting peptides are inserted in a hypervariable region of the AAV capsid sequence. Non-limiting examples of such hypervariable regions include Loop I, Loop IV, Loop VI, and Loop VIII of the parent AAV capsid. While not wishing to be bound by theory, these surface exposed loops, which may hereinafter also be referred to as surface loops, are unstructured and poorly conserved, making them ideal regions for insertion of targeting peptides.

In some embodiments, the targeting peptide is inserted into Loop I. In another embodiment, the targeting peptide is used to replace a portion, or all of Loop I. As a non-limiting example, addition of the targeting peptide to the parent AAV capsid sequence may result in the replacement or mutation of at least one amino acid of the parent AAV capsid.

In some embodiments, the targeting peptide is inserted into Loop IV. In another embodiment, the targeting peptide is used to replace a portion, or all of Loop IV. As a non-limiting example, addition of the targeting peptide to the parent AAV capsid sequence may result in the replacement or mutation of at least one amino acid of the parent AAV capsid.

In some embodiments, the targeting peptide is inserted into Loop VI. In another embodiment, the targeting peptide is used to replace a portion, or all of Loop VI. As a non-limiting example, addition of the targeting peptide to the parent AAV capsid sequence may result in the replacement or mutation of at least one amino acid of the parent AAV capsid.

In some embodiments, the targeting peptide is inserted into Loop VIII. In another embodiment, the targeting peptide is used to replace a portion, or all of Loop VIII. As a non-limiting example, addition of the targeting peptide to the parent AAV capsid sequence may result in the replacement or mutation of at least one amino acid of the parent AAV capsid.

In some embodiments, more than one targeting peptide is inserted into a parent AAV capsid sequence. As a non-limiting example, targeting peptides may be inserted at both Loop IV and Loop VIII in the same parent AAV capsid sequence.

Targeting peptides may be inserted at any amino acid position of the parent AAV capsid sequence, such as, but not limited to, between amino acids at positions 586-592, 588-589, 586-589, 452-458, 262-269, 464-473, 491-495, 546-557 and/or 659-668.

In a preferred embodiment, the targeting peptides are inserted into a parent AAV capsid sequence between amino acids at positions 588 and 589 (Loop VIII). In some embodiments, the parent AAV capsid is AAV9 (SEQ ID NO: 2). In a second embodiment, the parent AAV capsid is K449R AAV9 (SEQ ID NO: 3).

In some embodiments, the targeting peptides are inserted into a parent AAV capsid sequence between amino acids at positions 454, 455, 457, 457, 458, 459, 460, and/or 461 (Loop IV).

In some embodiments, the targeting peptides are inserted into a parent AAV capsid sequence between amino acids at positions 586, 587, 588, 589, and/or 590 (Loop VIII).

In some embodiments, the targeting peptides are inserted into a parent AAV capsid sequence Loop IV. As a non-limiting example, the parent AAV capsid is the AAV5 capsid sequence.

In some embodiments, the targeting peptides are inserted into a parent AAV capsid sequence Loop VIII. As a non-limiting example, the parent AAV capsid is the AAV5 capsid.

The targeting peptides described herein may increase the transduction of the AAV particles of the disclosure to a target tissue as compared to the parent AAV particle lacking a targeting peptide insert. In some embodiments, the targeting peptide increases the transduction of an AAV particle to a target tissue by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In some embodiments, the targeting peptide increases the transduction of an AAV particle to a cell or tissue of the CNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In some embodiments, the targeting peptide increases the transduction of an AAV particle to a cell or tissue of the PNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In some embodiments, the targeting peptide increases the transduction of an AAV particle to a cell or tissue of the DRG by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

### Promoters

AAV particles comprising the novel capsids defined by the present disclosure, which may hereinafter also be referred to as TRACER AAV particles, may optionally comprise at least one element to enhance target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In some embodiments, the promoter is deemed to be efficient when it drives expression of the polypeptide(s) encoded by AAV capsid mRNA described herein. In some embodiments, the promoter is deemed to be efficient when it drives expression of the polypeptide(s) encoded by viral genomes encapsulated within a capsid described herein.

In some embodiments, the promoter drives expression of the polypeptides (e.g., AAV capsid polypeptides) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years, Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiments, the promoter drives expression of the polypeptides (e.g., AAV capsid polypeptides) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA; such as, but not limited to, a CBA promotor as described in Miyazaki et al. (Gene. 1989 Jul. 15; 79(2):269-77, the contents of which are herein incorporated by reference in its entirety)) and its derivative CAG, pi glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes. Non-limiting examples of promotors are listed in Table 2.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety). Muscle specific promotors may also include Mb promoter, myosin promotor, dystrophin promotor, dMCK and tMCK. As a non-limiting example, the muscle-specific promotor may be used to drive or promote expression in certain cell types, such as, but not limited to, myocytes and muscle stem cells.

Non-limiting examples of blood-specific promoters include B29 promoter, immunoglobulin heavy chain promoter, CD45 promoter, mouse INF-β promoter, CD45 SV40/CD45 promoter, WASP promoter, CD43 promoter. CD43 SV40/CD43 promoter, CD68 promoter, GPIIb promoter, CD14 promoter, and CD2 promoter. As a non-limiting example, the blood-specific promotor may be used to drive or promote expression in certain cell types, such as, but not limited to, in B cells, hematopoietic cells, leukocytes, platelets, macrophages, megakaryocytes, monocytes and/or T cells.

Non-limiting examples of bone-specific promotors include osteocalcin, bone sialoprotein, and OG-2 promoter. As a non-limiting example, the bone-specific promotor may be used to drive or promote expression in certain cell types, such as, but not limited to, osteoblasts and odontoblasts.

Non-limiting examples of eye-specific promotors include Chx10, PrP, Dkk3, Math5, Ptf1a, Pcp2, Nefh, gamma-synuclein gene (SNCG), Grik4, Pdgfra, Chat, Thy1.2, hVmd2, Thy1, Modified αA-crystallin, hRgp, mMo, Opn4, RLBP1, Glast, Foxg1, hVmd2, Trp1, Six3, ex36, Grm6-SV40 eukaryotic promoter, hVmd2, Dct Rpc65, mRho, Irbp, hRho, Pcp2, Rhodopsin, and mSo, As a non-limiting example, the eye-specific promotor may be used to drive or promote expression in certain cell types, such as, but not limited, to retinal neurons, horizontal cells, bipolar cells, ganglion cells (GCs), ONL Müller cells, amacrine cells, lens cells, S-cone cells, M-cone cells, melanopsin-expressing GCs, neurons, ON bipolar, optic nerve cells, pigmented cells, retinal pigment epithelial cells, rod cells, rod bipolar cells, and rod photoreceptors.

Non-limiting examples of heart-specific promotors include MLC2v promoter, αMHC promoter, rat troponin T (Tnnt2), Tie2, and Tcf21. As a non-limiting example, the heart-specific promotor may be used to drive or promote expression in certain cell types, such as, but not limited to, cardiomyocytes, endothelial cells, and fibroblasts.

Non-limiting examples of kidney-specific promotors include, ECAD, NKCC2, KSPC, NPHS1, and SGLT2. As a non-limiting example, the kidney-specific promotor may be used to drive or promote expression in certain cell types, such as, but not limited to, collecting duct cells, loop of Henle cells, nephron cells, podocytes and proximal tubular cells.

Non-limiting examples of liver-specific promotors include, SV40/bAlb promoter, SV40/hAlb promoter, Hepatitis B virus core promoter, and Alpha fetoprotein. As a non-limiting example, the liver-specific promotor may be used to drive or promote expression in certain cell types, such as, but not limited to, hepatocytes.

Non-limiting examples of lung-specific promotors include Surfactant protein B promoter and Surfactant protein C promoter. As a non-limiting example, the lung-specific promotor may be used to drive or promote expression in certain cell types, such as, but not limited to, AT II cells and Clara cells.

Non-limiting examples of pancreas-specific promotors include elastase-1 promoter, PDX1 promoter, and insulin promoter. As a non-limiting example, the pancreas-specific promotor may be used to drive or promote expression in certain cell types, such as, but not limited to, acinar cells, beta cells, and Langerhans cells.

Non-limiting examples of vascular- or vasculature-specific promotors include Slco1c1, tie, cadherin, ICAM-2, claudin 1, Cldn5, Flt-1 promoter, and Endoglin promoter. As a non-limiting example, the vascular-specific promotor may be used to drive or promote expression in certain cell types, such as, endothelial cells. As a non-limiting example, the endothelial cell is a blood-brain barrier endothelial cell.

Non-limiting examples of tissue-specific expression elements for neurons include neuron-specific enolase (NSE), platelet-derived growth factor (PDGF), platelet-derived growth factor B-chain (PDGF-β), synapsin (Syn or Syn1), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH) chain, β-globin minigene nβ2, preproenkephalin (PPE), enkephalin (Enk), VGF, and excitatory amino acid transporter 2 (EAAT2) promoters. A non-limiting examples of tissue-specific expression elements for neuroectodermal stem cells is nestin.

Non-limiting examples of tissue-specific expression elements for astrocytes include glial fibrillary acidic protein (GFAP, GFabc1D) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the TRACER AAV particle comprises a ubiquitous promoter. Non-limiting examples of ubiquitous promoters include CMV, CBA (including derivatives CAG, CB6, CBh, etc.), EF-1α, PGK, UBC, GUSB (hGBp), and UCOE (promoter of HNRPA2B1-CBX3).

Yu et al. (Molecular Pain 2011, 7:63: the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HβH construct with a hGUSB promoter, an HSV-1LAT promoter and an NSE promoter and found that the HOH construct showed weaker expression than NSE in mouse brain. Passini and Wolfe (J. Virol. 2001, 12382-12392, the contents of which are herein incorporated by reference in its entirety) evaluated the long-term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920-nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus, and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes. Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews, or Raymond may be used in the present disclosures.

In some embodiments, the promoter is ubiquitous.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a β-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides.

In some embodiments, the promoter is a neurofilament heavy (NFH) promoter. The NFH promoter may have a size of 900-950 nucleotides. As a non-limiting example, the NFH promoter is 920 nucleotides.

In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken p-actin (CBA) promoter, or a variant thereof.

In some embodiments, the promoter is a CB6 promoter.

In some embodiments, the promoter is a minimal CB promoter.

In some embodiments, the promoter is a P40 promoter. In some embodiments, the P40 promoter is located in the 3′ of the AAV capsid REP gene.

In some embodiments, the promoter is a cytomegalovirus (CMV) promoter.

In some embodiments, the CMV promoter is a hybrid CMV enhancer/Chicken beta-actin promoter sequence such as described by Niwa et al., 1991, the contents of which are incorporated herein by reference in their entirety.

In some embodiments, the promoter is a CAG promoter.

In some embodiments, the promoter is a GFAP promoter.

In some embodiments, the promoter is a synapsin (syn or syn1) promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In some embodiments, the promoter is an RNA pol III promoter. As a non-limiting example, the RNA pol III promoter is U6. As a non-limiting example, the RNA pol III promoter is H1.

In some embodiments, the promoter may be selected depending on the desired tropism. Examples of such promoters are found in Table 2.

In some embodiments, the promoter drives capsid mRNA expression in the absence of helper virus co-infection.

In some embodiments, the TRACER AAV particle comprises two promoters. As a non-limiting example, the promoters are an P40 promoter and a CMV promoter. As another non-limiting example, the promoters are an P40 promoter and a cell-type specific promoter (e.g. synapsin).

In some embodiments, the TRACER AAV particle comprises an engineered promoter.

In another embodiment, the TRACER AAV particle comprises a promoter from a naturally expressed protein.

In some embodiments, a portion of the TRACER AAV particle REP gene is deleted to accommodate the promoter insertion. The promoter may be inserted upstream or downstream of the TRACER AAV particle CAP gene.

In some embodiments, the TRACER AAV particles of the present disclosure comprise a cell type-specific promoter to drive capsid mRNA expression. As a non-limiting example, the promotor is cell-type specific. The cell-type specific promotor may be synapsin. The cell-type specific promotor may be glial fibrillary acidic protein (GFAP). The TRACER AAV particle may comprise a P40 promoter and a cell-type specific promotor.

### AAV Production

Viral production disclosed herein describes processes and methods for producing AAV particles (with enhanced, improved and/or increased tropism for a target tissue) that may be used to contact a target cell to deliver a payload.

The present disclosure provides methods for the generation of AAV particles comprising targeting peptides. In some embodiments, the AAV particles are prepared by viral genome replication in a viral replication cell. Any method known in the art may be used for the preparation of AAV particles. In some embodiments, AAV particles are produced in mammalian cells (e.g., HEK293). In another embodiment, AAV particles are produced in insect cells (e.g., Sf9).

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, N J (1995); O'Reilly et al., Baculovirus Expression Vectors, A Laboratory Manual, Oxford Univ. Press (1994); Samulski et al., J Vir. 63:3822-8 (1989); Kajigaya et al., Proc. Nat'l. Acad. Sci. USA 88: 4646-50 (1991); Ruffing et al., J Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in International Patent Publication WO2015191508, the contents of which are herein incorporated by reference in their entirety.

### AAV Selection

The present disclosure provides methods of AAV selection for tissue- and/or cell type-specific transduction, whereby TRACER AAV particles with high tropism for a tissue(s) and/or cell type(s) are identified and selected for use. In some embodiments, the TRACER method used may be, in whole, or in part, as described in WO2020072683 or Nonnenmacher et al., Mol Ther Methods Clin Dev. 20:366-378 (2020), the contents of each of which are herein incorporated by reference in their entirety. In some embodiments, the AAV selection comprises administration of the TRACER AAV particles to a cell and/or a subject by standard methods known in the art (e.g. intravenously). In some embodiments, the AAV selection may comprise extraction of polynucleotides, e.g., capsid RNA, encoded by TRACER AAV particles, from a specific tissue and/or cell type. In some embodiments, the tissue may be non-nervous system tissue such as, but not limited to, liver, spleen and heart. The cells type may be, e.g., hepatocytes, Islets of Langerhans cells, and cardiomyocytes. In some embodiments, the tissue may be nervous system tissue such as, but not limited to, brain tissue, spinal cord tissue, and dorsal root ganglion tissue. The cell type may be, e.g., neurons, astrocytes, or oligodendrocytes. In some embodiments, the extracted RNA is enriched, reverse transcribed, and/or amplified. In some embodiments, the extracted RNA allows for recovery of full-length capsid “amplicon(s)” from a specific tissue and/or cell type, using various production methods, e.g., reverse transcription polymerase chain reaction (RT-PCR). As used herein, amplicon may refer to any piece of RNA or DNA formed as the product of amplification events, e.g. PCR. In some embodiments, full-length capsid amplicons may be used as templates for next generation sequencing (NGS) library generation. Full-length capsid amplicons may be used for cloning into a DNA library for the generation of AAV TRACER particles for any number of additional rounds of AAV selection as described above. In some embodiments, the AAV selection may be performed iteratively, or repeated, any number of times, or rounds. The above-described selection of AAV TRACER particles may also be more generally referred to herein as biopanning. As used herein, “biopanning” refers to an AAV capsid library selection process comprising administration of an AAV particle with enhanced tissue- and/or cell type-specific transduction to a cell and/or subject; extraction of nucleotides encoded by said AAV particle from said transduced tissue- and/or cell type-specific; and, use of the extracted nucleotides for cloning into a nucleotide library for the generation of AAV particles for subsequent rounds of the same.

In some embodiments, the AAV selection comprises administration of the TRACER AAV particles to a cell by standard methods known in the art (e.g. infection). As a non-limiting example, the cell is a HEK293 cell. As another non-limiting example, the cell is a nervous system cell such as, but not limited to, a neuron and/or a glial cell. As yet another non-limiting example, the cell is a brain microvascular endothelial cells (BMVEC). The BMVEC may be a human BMVEC (hBMVEC). The BMVEC may be a non-human primate (NHP) BMVEC.

In some embodiments, the AAV selection comprises administration of the TRACER AAV particles to a rodent by standard methods known in the art (e.g. intravenously). The rodent may be a transgenic rodent or a non-transgenic (i.e., wild type) rodent. As a non-limiting example, the rodent is a rat or a mouse. Non-limiting examples of rats include Sprague Dawley, Wistar Albino, and Long Evans rats. Non-limiting examples of mice include BALB/C, FVB and C57BL/6 mice.

In some embodiments, the AAV selection comprises administration of the TRACER AAV particles to a non-human primate (NHP) by standard methods known in the art (e.g. intravenously). Non-limiting examples of NHPs include rhesus macaques (Macaca mulatta) and cynomolgus macaques (Macaca fascicularis).

In some embodiments, the AAV selection comprises administration of TRACER AAV particles to a rodent, non-human primate, and/or human cells. In some embodiments, the AAV selection comprises administration of TRACER AAV particles to a rodent, non-human primate, and/or human subjects.

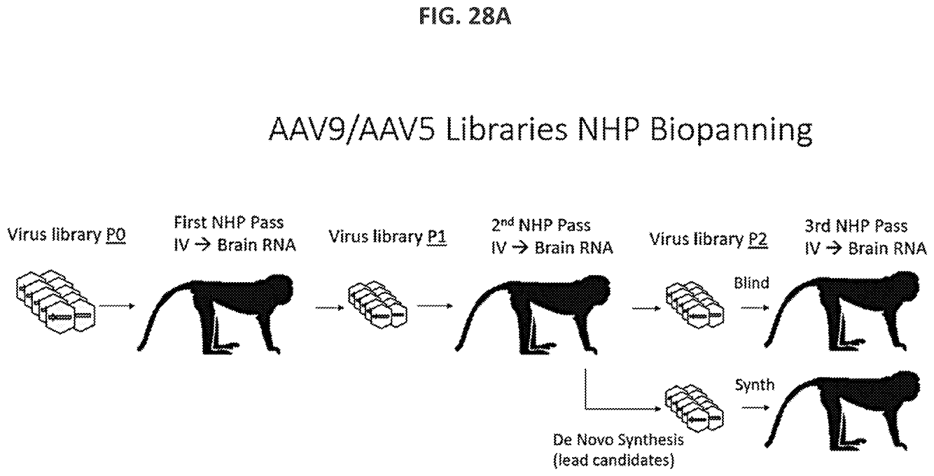

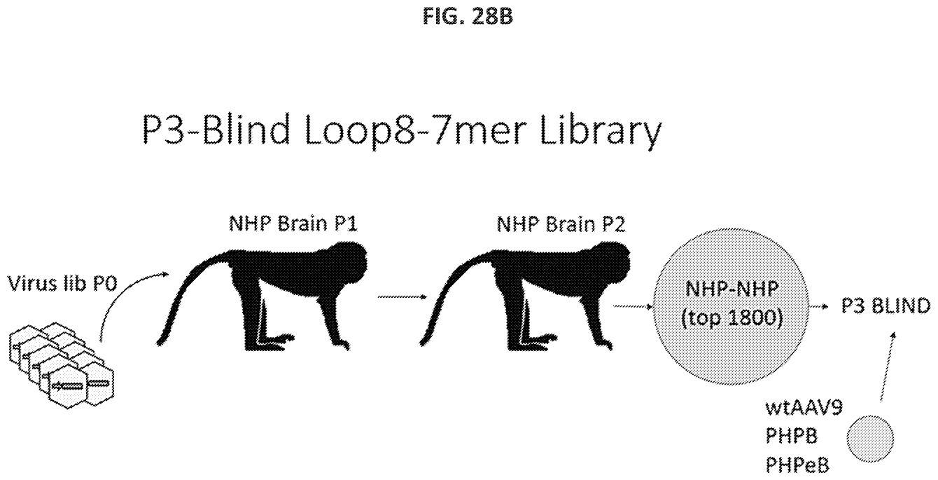

In some embodiments, the AAV selection may be performed iteratively, or repeated, any number of times, or rounds, within a single cell- or subject-type, wherein the single cell- or subject-type may remain unchanged, or the same, across AAV selection rounds. Cell types may be, e.g., HEK293 cells, hBMVECs, and NHP BMVECs. Subject types may be, e.g., Sprague Dawley rats, Wistar Albino rats, Long-Evans rats, BALB/C mice, FVB mice, C57BL/6 mice, rhesus macaques, cynomolgus macaques, and humans. As a non-limiting example, AAV selection is performed across one, two and/or three or more AAV selection rounds in the hBMVEC cell. As a non-limiting example, AAV selection is performed across one, two and/or three or more rounds in a mouse such as, but not limited to, a BALB/C mouse. As a non-limiting example, AAV selection is performed across one, two and/or three or more rounds in an NHP such as, but not limited to, a cynomolgus macaque, as represented in FIG. 28A and FIG. 28B.

AAV selection may be performed iteratively, or repeated, any number of times, or rounds, within any number of cell- and/or subject-types, wherein the cell- and or subject-type may change, or differ, across AAV selection rounds. As a non-limiting example, the AAV selection is performed a first round in a rhesus macaque, and an additional, i.e., subsequent, one, two, and/or or three or more rounds in a Sprague-Dawley rat.

AAV selection may be performed iteratively, or repeated, any number of times, or rounds, within any number of cell- and/or subject-types, and may additionally comprise the combination and/or comparison of any AAV capsid serotype as disclosed herein, or variants or derivatives thereof, with the TRACER AAV particle pool, at any AAV selection round. As a non-limiting example, the AAV capsid serotype comprises AAVF7/HSC7 (SEQ ID NO: 8 and 27 of WO2016049230). As another non-limiting example, the AAV capsid serotype comprises AAVF15/HSC15 (SEQ ID NO: 16 and 33 of WO2016049230). As yet another non-limiting example, the AAV capsid serotype AAVF17/HSC17 (SEQ ID NO: 13 and 35 of WO2016049230). The AAV selection round may be the first, second, third, or fourth AAV selection round.

### Orthogonal Evolution

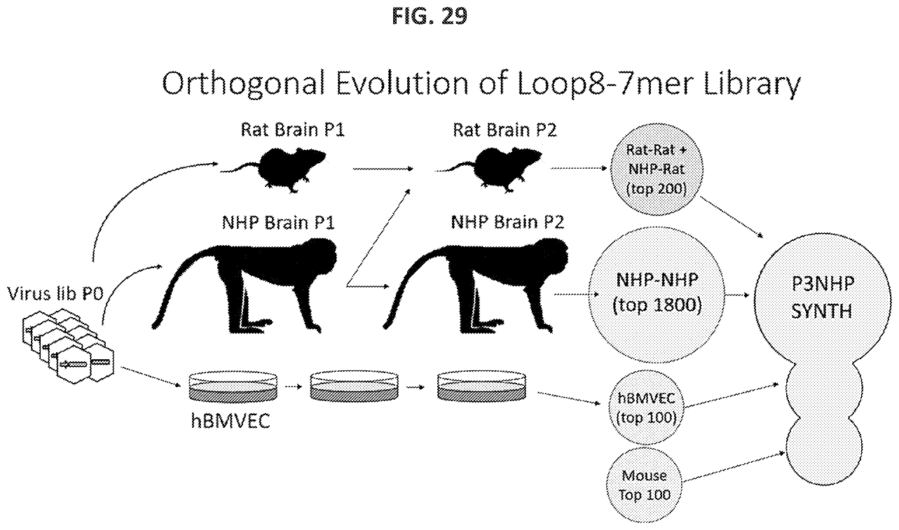

Methods of AAV selection of the present disclosure may comprise orthogonal evolution. As used herein, “orthogonal evolution” refers to a method wherein AAV particles are administered for a first round of AAV selection as described herein across a set of any number of cell- and/or subject-types that may be from different species and/or strains, and wherein any number of additional, i.e., subsequent, AAV selection rounds are performed either across a set of any number of cell- and/or subject-types that may be from different species and/or strains, or across a set of any number of cell- and/or subject-types that may be from the same species and/or strains, as represented in FIG. 29. Cell types may be, e.g., HEK293 cells, hBMVECs, and NHP BMVECs. Subject types may be, e.g., Sprague Dawley rats, Wistar Albino rats, Long-Evans rats, BALB/C mice, FVB mice, C57BL/6 mice, rhesus macaqucs, cynomolgus macaques, and humans.

### Combination

Compositions, methods, processes for the preparation, and/or use of TRACER AAV particles of the present disclosure may be used in combination with one or more other (additional) therapeutic, prophylactic, research or diagnostic agents and/or methods. By “in combination with,” it is not intended to imply that the other agents and/or methods must be used at the same time as TRACER AAV particle agents and/or methods described herein, although this combination is within the scope of the present disclosure. In combination with may refer to the use of one or more other (additional) agents and/or methods that may occur concurrently with, prior to, or subsequent to any TRACER AAV particle agents and/or methods described herein. As a non-limiting example, the present disclosure encompasses TRACER AAV particle methods of the present disclosure used in combination with one or more other methods such as, but not limited to, continuous evolutions method (e.g., phage-assisted continuous evolution or PACE) described herein.

### Phage-Assisted Continuous Evolution (PACE)

In some embodiments, methods of TRACER AAV particles may be used in combination with methods of continuous evolutions such as, but not limited, to phage-assisted continuous evolution (PACE). PACE is a continuous evolution method in which phage-based libraries are evolved over short periods of time with genomes that express proteins that may interact with a designated target (e.g. a promoter). Methods of PACE are described in Esvelt et al., (Nature, 477(7344): 499-503 (2011)), Leconte et al. (Biochemistry. 52(8):1490-9 (2013)), Fu et al. (Recent Pat DNA Gene Seq. 7(2):144-56 (2013)), Dickinson et al. (Proc Natl Acad Sci USA. 110(22):9007-12 (2013)), Carlson et al. (Nat Chem Biol.; 10(3):216-22 (2014)), Dickinson et al. (Nat Commun 5:5352 (2014)), Hubbard et al. (Nat Methods. 12(10):939-42 (2015)), Badran et al. (Nature. 533(7601):58-63 (2016)), Pu et al. (Nat Chem Biol. 13(4):432-438 (2017)), Bryson et al. (Nat Chem Biol. 13(12):1253-1260 (2017)), Suzuki et al. (Nat Chem Biol. 13(12):1261-1266 (2017)), Packer et al. (Nat Commun. 8(1):956 (2017)), Hu et al. (Nature. 556(7699):57-63 (2018)), Wang et al. (Nat Chem Biol. 14(10):972-980 (2018)), Shaver et al. (BMC Res Notes. 11(1):861 (2018)), Li et al. (J Microbiol Biotechnol. doi: 10.4014/jmb.1810.10060 (2019)), Roth et al. (ACS Synth Biol. 8(4):796-806 (2019)), and Thuronyi et al. (Nat Biotechnol. 37(9):1070-1079 (2019)), and in U.S. Pat. Nos. 9,023,594, 10,336,997, 9,790,531, and 10,392,674, and in US Publication Nos. US20190256842, US20170233708, US20180142031. US20180087046, US20180057545, US20180237758, US20180187182, US20190218532, and US20190153472, and in International Publication Nos. WO2016US43691, WO2017212400, WO2018136939, WO2019010164, WO2019023680, WO2019033095, WO2019040935, WO2019056002, WO2019125804, WO2019161251, and WO2019168953, the contents of each of which are herein incorporated by reference in their entirety. To evolve a library for PACE studies, genes with a desired function may be linked to the production of phage components needed for replication. As a result, the PACE system may select for vectors carrying the genes with desired functionality by creating an environment in which said vectors are more likely to replicate than those lacking genes with desired functionality.

PACE may be conducted with a plurality of host cells. Any cell (e.g. bacterial, fungal, insect, human, mammalian, plant, protozoan, etc.) may be used as a host cell. In some embodiments, host cells may be bacterial. Bacterial host cells may include Escherichia coli (E. coli). Additional plasmids for the PACE process (e.g. accessory plasmids, helper plasmids, and/or mutagenesis plasmids) may also be inserted into host cells.

During PACE, phage vectors termed selection phages may be prepared from phage or phagemid. As used herein, the term “selection phage” or “SP” refers to a phage vector, including but not limited to bacteriophage, phage, phagemid, and/or phage particle, that may be modified for evolution of a protein and/or nucleic acid library. Selection phage may be modified to encode members of a nucleic acid-based library. Selection phage may be further modified to lack a propagation component gene. In some embodiments, selection phage may include insertions of up to 42 kb into the phage genome. Non-limiting examples of selection phages may include M13 filamentous bacteriophage 11, VCSM13 helper phage, HP-T7RNAP A, and any other phage, phagemid, and/or phage particle described in Esvelt et al., (Nature, 472(7344): 499-503 (2011)), Leconte et al. (Biochemistry. 52(8):1490-9 (2013)), Fu et al. (Recent Pat DNA Gene Seq. 7(2):144-56 (2013)), Dickinson et al. (Proc Natl Acad Sci USA. 110(22):9007-12 (2013)). Carlson et al. (Nat Chem Biol.; 10(3):216-22 (2014)), Dickinson et al. (Nat Commun 5:5352 (2014)), Hubbard et al. (Nat Methods. 12(10):939-42 (2015)), Badran et al. (Nature. 533(7601):58-63 (2016)), Pu et al. (Nat Chem Biol. 13(4):432-438 (2017)), Bryson et al. (Nat Chem Biol. 13(12):1253-1260 (2017)). Suzuki et al. (Nat Chem Biol. 13(12):1261-1266 (2017)), Packer et al. (Nat Commun. 8(1):956 (2017)), Hu et al. (Nature. 556(7699):57-63 (2018)), Wang et al. (Nat Chem Biol. 14(10):972-980 (2018)), Shaver et al. (BMC Res Notes. 11(1):861 (2018)), Li et al. (J Microbiol Biotechnol. doi: 10.4014/jmb.1810.10060 (2019)), Roth et al. (ACS Synth Biol. 8(4):796-806 (2019)), and Thuronyi et al. (Nat Biotechnol. 37(9):1070-1079 (2019)), and in U.S. Pat. Nos. 9,023,594, 10,336,997, 9,790,531, and 10,392,674, and in US Publication Nos. US20190256842, US20170233708, US20180142031, US20180087046, US20180057545, US20180237758, US20180187182, US20190218532, and US20190153472, and in International Publication Nos. WO2016US43691, WO2017212400, WO2018136939, WO2019010164, WO2019023680, WO2019033095, WO2019040935, WO2019056002, WO2019125804, WO2019161251, and WO2019168953, the contents of each of which are herein incorporated by reference in their entirety.

For PACE studies, selection phage may be prepared such that it lacks a “propagation component gene”, a functional gene required for host infection. Propagation component genes may be any gene that encodes a protein able to facilitate host infection. Non-limiting examples of propagation component genes include gI, gII, gIII, gIV, gV, gVI, gVII, gVIII, gIX, gX, and combinations thereof. Selection phage without propagation component genes may be prepared by any method known to one of skill in the art (e.g. recombinase-mediated inversion and/or riboswitches that may be optionally small molecule dependent). In some embodiments, propagation component genes may be gene III (gIII) a gene that encodes gene III protein (pIII), a protein that facilitates phage infection of bacterial hosts via binding with F pilus. Instead of inclusion in the selection phage, propagation component genes (e.g. gIII) may be added to an “accessory plasmid”, a plasmid containing a functional gene required for viral replication which has been removed and/or altered in the selection phage. Accessory plasmids, as well as plasmids and/or phagemids encoding all phage proteins excluding the functional gene for viral replication (“helper plasmids” or “helper phagemids”), may be incorporated into the host cell population.

In some embodiments, host cells may be flowed through a vessel of fixed-volume, known as a “lagoon”. Lagoons may also contain a replicating population selection phage. Host cells may flow through a lagoon at a rate such that the residence time of host cells may be shorter than that of host cell replication, but longer than that of replication of selection phage (Esvelt et al. Nature, 472(7344): 499-503 (2011); U.S. Pat. No. 9,023,594). This flow rate may ensure that accretion of mutations, and therefore evolution, occurs in the phage population and few or no effects are seen by the host cell population.

Selection phage encoding library members may infect host cells in the lagoon. In some embodiments, expression of propagation component genes on accessory plasmids may be reliant on expression of genes from selection phage. In some embodiments, selection phage encoding genes able to generate expression of propagation component genes on accessory plasmids may be capable of generating infectious progeny and may be able to propagate. Production of infectious phage may be directly proportional to the amount of propagation component gene (e.g. gIII) expressed. Selection phage that do not generate expression of propagation component genes may be non-functional library members. Non-functional library members may fail to propagate and/or produce infection progeny, and they may wash out of the lagoon. Infectious phage may include functional library members, which may continue the cycle of propagation and evolve in the lagoon. Selection phage remaining at the end of PACE may be isolated, sequenced, and/or characterized via any method known to one of skill in the art to identify the evolved gene.

In some embodiments, accessory plasmids include promoters upstream of propagation component genes (e.g. a promoter upstream from gIII). In some embodiments, promoters are constantly active. In some embodiments, promoters are conditionally active such that they are active only when defined criteria are met (e.g. the presence of an activating agent, such as a protein linking the promoter to other transcription machinery). In some embodiments, the defined criterion needed to activate a conditionally active promoter may be the presence and/or binding of a small molecule. Such promoters may be considered induced in the presence of these small molecules (e.g. arabinose). In some embodiments, propagation component genes on accessory plasmids may be controlled via conditionally active promoters. In some embodiments, selection phage encodes elements, features, nucleic acids, and/or proteins that may interact with conditionally active promoters and activate transcription of the propagation component gene. These conditionally active promoters may serve as targets for the selection of interacting elements to drive evolution. Selection phages encoding genes interacting with a described target may be functional library members that enable the expression of the components needed for selection phage to become infectious. As a result, these selection phages may display enhanced infectiousness, and continue to infect host cells, replicate, and evolve in the lagoon, while others do not replicate and wash out. In some embodiments, rounds of replication of functional library members may result in the evolution of library members with genes encoding the desired functionality (e.g., genes encoding an optimized element, feature, nucleic acid, and/or protein for activation of conditionally active promoters of propagation component genes on accessory plasmids) (Esvelt et al. Nature, 472(7344): 499-503 (2011); Hu et al. Nature. 556(7699):57-63 (2018); U.S. Pat. Nos. 9,023,594, 10,392,674). Selection phage remaining at the end of PACE may be isolated, sequenced, and/or characterized via any method known to one of skill in the art to identify the evolved gene. Activity of the evolved genes, and subsequent evolved proteins, may be verified by any method known to one of skill in the art.

In some embodiments, evolutionary pressure may be tuned within a PACE system. In some embodiments, host cells may further include mutagenesis plasmids which may increase the error rate during DNA replication in the lagoon and enhance the rate of mutation, as described in Esvelt et al. Nature, 472(7344): 499-503 (2011). Mutagenesis plasmids may be inducible via a small molecule (e.g. arabinose). In some embodiments, the evolutionary pressure may be tuned via control of the copy number of accessory plasmids. In some embodiments, evolutionary pressure may be tuned via modulation of ribosome binding sites (RBS) on propagation component genes.

In some embodiments, selection stringency of the PACE system may be controlled via modulation of the conditionally active promoter serving as the target for selection. Starting selection phage libraries may not encode elements initially able to interact with target conditionally active promoter. Consequently, initial series of PACE may be conducted against a hybrid conditionally active promoter, followed by series of PACE against target conditionally active promoter, as described in Esvelt et al. Nature, 472(7344): 499-503 (2011). In some embodiments, selection stringency may be tuned via further modulation of expression of propagation component genes, as described in Carlson et al. (Nat Chem Biol.; 10(3):216-22 (2014) and Leconte et al. (Biochemistry. 52(8):1490-9 (2013). In some embodiments, different promoters may be used to control expression of propagation component genes. In some embodiments, additional copies of propagation component genes may be added to accessory plasmids with small molecule inducible promoters (e.g. Tet-inducible promoters), enabling small molecule concentration-based control of propagation component gene expression, which may be independent of library evolution. Higher concentrations of small molecule may lead to increased expression of propagation component genes independent of the genes expressed by selection phage. When expression of propagation component genes is increased, a greater amount of selection phage may continue to replicate in the lagoon, reducing stringency and keeping a larger library size for a longer period of time. Decreased concentrations of small molecule may reduce independent expression of propagation component genes, increasing stringency and reducing library size.

In some embodiments, these additional copies of propagation component genes may be included in the same accessory plasmid in which propagation component gene expression is linked to evolution. In some embodiments, these additional copies of propagation component genes may be included on a different accessory plasmid or any other plasmid described herein (e.g., a mutagenesis plasmid and/or helper plasmid). Plasmids including cassettes with these additional copies of propagation component genes for the control of selection stringency may be referred to as “drift plasmids”.

In some embodiments, PACE methods may include methods of negative selection, as described in Carlson et al. (Nat Chem Biol.; 10(3):216-22 (2014) and in U.S. Pat. No. 10,392,674 and in US Publication No. US20190256842. PACE methods with negative selection may select against genes in selection phage with unwanted activity. In some embodiments, PACE methods with negative selection may enable the evolution of elements, features, nucleic acids, and/or proteins that selectively generate a preferred outcome (e.g. proteins selective for one interacting partner over another). In some embodiments, PACE methods may include host cells with a first accessory plasmid. This first accessory plasmid may include at least one propagation component gene. The first accessory plasmid may further include a conditionally active promoter upstream of the at least one propagation component gene. In some embodiments, PACE methods may include host cells with a second accessory plasmid. Secondary accessory plasmids may encode a dominant negative version of a propagation component gene (e.g. a dominant negative version of pIII). The second accessory plasmid may further include a small molecule inducible promoter (e.g. an IPTG inducible promoter) upstream from the dominant negative version of a propagation component gene. Expression of the dominant negative version of said propagation component gene may be controlled via concentration of small molecule (e.g. IPTG).

In some embodiments, any of the described TRACER AAV particle compositions, methods, processes for preparation, and/or use may be used in combination with any of the described PACE compositions, methods, processes for preparation, and/or use.

In some aspects, any of the described TRACER AAV particle compositions, methods, processes for preparation, and/or use may be used concurrently with any of the described PACE compositions, methods, processes for preparation, and/or use.

In some aspects, any of the described TRACER AAV particle compositions, methods, processes for preparation, and/or use may be used prior to any of the described PACE compositions, methods, processes for preparation, and/or use.

In some aspects any of the described TRACER AAV particle compositions, methods, processes for preparation, and/or use may be used subsequent to any of the described PACE compositions, methods, processes for preparation, and/or use.

Any of the described TRACER AAV particle compositions, methods, processes for preparation, and/or use may be used a single time or iteratively any number of times, i.e. repeated, in combination with any of the described PACE compositions, methods, processes for preparation, and/or use used a single time. Any of the described TRACER AAV particle compositions, methods, processes for preparation, and/or use may be used a single time or iteratively any number of times in combination with any of the described PACE compositions, methods, processes for preparation, and/or use used iteratively.

In some embodiments, any of the described TRACER AAV particle compositions, methods, processes for preparation, and/or use may be used a single time or iteratively any number of times, in combination with any of the described PACE compositions, methods, processes for preparation, and/or use used a single time or iteratively any number of times. In such embodiments, any of the described TRACER AAV particle compositions, methods, processes for preparation, and/or use may be used concurrently, prior to, or subsequent to any of the described PACE compositions, methods, processes for preparation, and/or use.

### Therapeutic Applications

The present disclosure provides a method for treating a disease, disorder and/or condition in a mammalian subject, including a human subject, comprising administering to the subject an AAV particle described herein where the AAV particle comprises the novel capsids (“TRACER AAV particles”) defined by the present disclosure or administering to the subject any of the described compositions, including pharmaceutical compositions, described herein.

In some embodiments, the TRACER AAV particles of the present disclosure are administered to a subject prophylactically, to prevent on-set of disease. In another embodiment, the TRACER AAV particles of the present disclosure are administered to treat (lessen the effects of) a disease or symptoms thereof. In yet another embodiment, the TRACER AAV particles of the present disclosure are administered to cure (eliminate) a disease. In another embodiment, the TRACER AAV particles of the present disclosure are administered to prevent or slow progression of disease. In yet another embodiment, the TRACER AAV particles of the present disclosure are used to reverse the deleterious effects of a disease. Disease status and/or progression may be determined or monitored by standard methods known in the art.

In some embodiments, the TRACER AAV particles of the disclosure are useful in the field of medicine for the treatment, prophylaxis, palliation or amelioration of neurological diseases and/or disorders.

In some embodiments, the TRACER AAV particles of the disclosure are useful in the field of medicine for the treatment, prophylaxis, palliation or amelioration of tauopathy.

In some embodiments, the TRACER AAV particles of the disclosure are useful in the field of medicine for the treatment, prophylaxis, palliation or amelioration of Alzheimer's Disease.

In some embodiments, the TRACER AAV particles of the disclosure are useful in the field of medicine for the treatment, prophylaxis, palliation or amelioration of Friedreich's ataxia, or any disease stemming from a loss or partial loss of frataxin protein.

In some embodiments, the TRACER AAV particles of the disclosure are useful in the field of medicine for the treatment, prophylaxis, palliation or amelioration of Parkinson's Disease.

In some embodiments, the TRACER AAV particles of the disclosure are useful in the field of medicine for the treatment, prophylaxis, palliation or amelioration of Amyotrophic lateral sclerosis.

In some embodiments, the TRACER AAV particles of the disclosure are useful in the field of medicine for the treatment, prophylaxis, palliation or amelioration of Huntington's Disease.

In some embodiments, the TRACER AAV particles of the disclosure are useful in the field of medicine for the treatment, prophylaxis, palliation or amelioration of chronic or neuropathic pain.

In some embodiments, the TRACER AAV particles of the disclosure are useful in the field of medicine for treatment, prophylaxis, palliation or amelioration of a disease associated with the central nervous system.

In some embodiments, the TRACER AAV particles of the disclosure are useful in the field of medicine for treatment, prophylaxis, palliation or amelioration of a disease associated with the peripheral nervous system.

In some embodiments, the TRACER AAV particles of the present disclosure are administered to a subject having at least one of the diseases or symptoms described herein.

As used herein, any disease associated with the central or peripheral nervous system and components thereof (e.g., neurons) may be considered a “neurological disease”.

Any neurological disease may be treated with the TRACER AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorca, Choreoacanthocytosis, Chronic Inflammatory Demyclinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myclinoclastic Diffuse Sclerosis, Myelitis, Myoclonic Encephalopathy of Infants, Myoclonus, Myoclonus epilepsy, Myopathy, Myopathy—Congenital, Myopathy—Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorca, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wemicke-KorsakoffSyndrome, West Syndrome, Whiplash, Whipple's Disease, Williams Syndrome, Wilson Disease, Wolman's Disease, X-Linked Spinal and Bulbar Muscular Atrophy.

### Methods of Treatment of Neurological Disease

### TRACER AAV Particles Encoding Protein Payloads

Provided in the present disclosure are methods for introducing the TRACER AAV particles of the present disclosure into cells, the method comprising introducing into said cells any of the vectors in an amount sufficient for an increase in the production of target mRNA and protein to occur. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed in the present disclosure are methods for treating neurological disease associated with insufficient function/presence of a target protein (e.g., ApoE, FXN) in a subject in need of treatment. The method optionally comprises administering to the subject a therapeutically effective amount of a composition comprising TRACER AAV particles of the present disclosure. As a non-limiting example, the TRACER AAV particles can increase target gene expression, increase target protein production, and thus reduce one or more symptoms of neurological disease in the subject such that the subject is therapeutically treated.

In some embodiments, the composition comprising the TRACER AAV particles of the present disclosure is administered to the central nervous system of the subject via systemic administration. In some embodiments, the systemic administration is intravenous (IV) injection.

In some embodiments, the composition comprising the TRACER AAV particles of the present disclosure is administered to the central nervous system of the subject via intraventricular administration. In some embodiments, the intraventricular administration is intra-cisterna magna injection (ICM).

In some embodiments, the composition comprising the TRACER AAV particles of the present disclosure is administered to the central nervous system of the subject via intraventricular injection and intravenous injection.

In some embodiments, the composition comprising the TRACER AAV particles of the present disclosure is administered to the central nervous system of the subject via ICM injection and intravenous injection at a specific dose per subject. As a non-limiting example, the TRACER AAV particles are administered via ICM injection at a dose of 1×10⁴ VG per subject. As a non-limiting example, the TRACER AAV particles are administered via IV injection at a dose of 2×10¹³ VG per subject.

In some embodiments, the composition comprising the TRACER AAV particles of the present disclosure is administered to the central nervous system of the subject. In other embodiments, the composition comprising the TRACER AAV particles of the present disclosure is administered to a CNS tissue of a subject (e.g., putamen, thalamus or cortex of the subject).

In some embodiments, the composition comprising the TRACER AAV particles of the present disclosure is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the TRACER AAV particles of the present disclosure is administered to the central nervous system of the subject via intraparenchymal injection and intravenous injection.

In some embodiments, the composition comprising the TRACER AAV particles of the present disclosure is administered to the central nervous system of the subject via intraventricular injection, intraparenchymal injection and intravenous injection.

In some embodiments, the TRACER AAV particles of the present disclosure may be delivered into specific types of targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In some embodiments, the TRACER AAV particles of the present disclosure may be delivered to neurons in the putamen, thalamus and/or cortex.

In some embodiments, the TRACER AAV particles of the present disclosure may be used as a therapy for neurological disease.

In some embodiments, the TRACER AAV particles of the present disclosure may be used as a therapy for tauopathies.

In some embodiments, the TRACER AAV particles of the present disclosure may be used as a therapy for Alzheimer's Disease.

In some embodiments, the TRACER AAV particles of the present disclosure may be used as a therapy for Amyotrophic Lateral Sclerosis.

In some embodiments, the TRACER AAV particles of the present disclosure may be used as a therapy for Huntington's Disease.

In some embodiments, the TRACER AAV particles of the present disclosure may be used as a therapy for Parkinson's Disease.

In some embodiments, the TRACER AAV particles of the present disclosure may be used as a therapy for Friedreich's Ataxia.

In some embodiments, the TRACER AAV particles of the present disclosure may be used as a therapy for chronic or neuropathic pain.

In some embodiments, administration of the TRACER AAV particles described herein to a subject may increase target protein levels in a subject. The target protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the protein levels of a target protein by at least 50%. As a non-limiting example, the TRACER AAV particles may increase the proteins levels of a target protein by at least 40%. As a non-limiting example, a subject may have an increase of 10% of target protein. As a non-limiting example, the TRACER AAV particles may increase the protein levels of a target protein by fold increases over baseline. In some embodiments, TRACER AAV particles lead to 5-6 times higher levels of a target protein.

In some embodiments, administration of the TRACER AAV particles described herein to a subject may increase the expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the expression of a target protein by at least 50%. As a non-limiting example, the TRACER AAV particles may increase the expression of a target protein by at least 40%.

In some embodiments, intravenous administration of the TRACER AAV particles described herein to a subject may increase the CNS expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may increase the expression of a target protein in the CNS by at least 50%. As a non-limiting example, the TRACER AAV particles may increase the expression of a target protein in the CNS by at least 40%.

In some embodiments, the TRACER AAV particles of the present disclosure may be used to increase target protein expression in astrocytes in order to treat a neurological disease. Target protein in astrocytes may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in microglia. The increase of target protein in microglia may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in cortical neurons. The increase of target protein in the cortical neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 540%, 545%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in hippocampal neurons. The increase of target protein in the hippocampal neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 540%, 545%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles may be used to increase target protein in DRG and/or sympathetic neurons. The increase of target protein in the DRG and/or sympathetic neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles of the present disclosure may be used to increase target protein in sensory neurons in order to treat neurological disease. Target protein in sensory neurons may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles of the present disclosure may be used to increase target protein and reduce symptoms of neurological disease in a subject. The increase of target protein and/or the reduction of symptoms of neurological disease may be, independently, altered (increased for the production of target protein and reduced for the symptoms of neurological disease) by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles of the present disclosure may be used to reduce the decline of functional capacity and activities of daily living as measured by a standard evaluation system such as, but not limited to, the total functional capacity (TFC) scale.

In some embodiments, the TRACER AAV particles of the present disclosure may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score). Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of neurological disease.

The TRACER AAV particles encoding the target protein may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the TRACER AAV particles of the present disclosure can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the TRACER AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the TRACER AAV particles of the present disclosure for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the TRACER AAV particle described herein may be co-administered with TRACER AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In some embodiments, administration of the TRACER AAV particles to a subject will increase the expression of a target protein in a subject and the increase of the expression of the target protein will reduce the effects and/or symptoms of neurological disease in a subject.

As a non-limiting example, the target protein may be an antibody, or fragment thereof.

### TRACER AAV Particles Comprising RNAi Agents or Modulatory Polynucleotides

Provided in the present disclosure are methods for introducing the TRACER AAV particles of the disclosure, comprising a viral genome with a nucleic acid sequence encoding one or more siRNA molecules into cells, the method comprising introducing into said cells any of the vectors in an amount sufficient for degradation of a target mRNA to occur, thereby activating target-specific RNAi in the cells. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed in the present disclosure are methods for treating neurological diseases associated with dysfunction of a target protein in a subject in need of treatment. The method optionally comprises administering to the subject a therapeutically effective amount of a composition comprising TRACER AAV particles comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the TRACER AAV particles of the present disclosure comprising a viral genome encoding one or more siRNA molecules comprise an AAV capsid that allows for enhanced transduction of CNS and/or PNS cells after intravenous administration.

In some embodiments, the composition comprising the TRACER AAV particles of the present disclosure with a viral genome encoding at least one siRNA molecule is administered to the central nervous system of the subject. In other embodiments, the composition comprising the TRACER AAV particles of the present disclosure is administered to a tissue of a subject (e.g., putamen, thalamus or cortex of the subject).

In some embodiments, the composition comprising the TRACER AAV particles of the disclosure, comprising a viral genome with a nucleic acid sequence encoding one or more siRNA molecules is administered to the central nervous system of the subject via systemic administration. In some embodiments, the systemic administration is intravenous injection.

In some embodiments, the composition comprising the TRACER AAV particles of the disclosure comprising a viral genome with a nucleic acid sequence encoding one or more siRNA molecules is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the TRACER AAV particles comprising a viral genome with a nucleic acid sequence encoding one or more siRNA molecules is administered to the central nervous system of the subject via intraparenchymal injection and intravenous injection.

In some embodiments, the TRACER AAV particles comprising a viral genome with a nucleic acid sequence encoding one or more siRNA molecules may be delivered into specific types or targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In some embodiments, the TRACER AAV particles comprising a viral genome with a nucleic acid sequence encoding one or more siRNA molecules may be delivered to neurons in the putamen, thalamus, and/or cortex.

In some embodiments, the TRACER AAV particles comprising a viral genome with a nucleic acid sequence encoding one or more siRNA molecules may be used as a therapy for neurological disease.

In some embodiments, the TRACER AAV particles comprising a viral genome with a nucleic acid sequence encoding one or more siRNA molecules may be used as a therapy for tauopathies.

In some embodiments, the TRACER AAV particles comprising a viral genome with a nucleic acid sequence encoding one or more siRNA molecules may be used as a therapy for Alzheimer's Disease.

In some embodiments, the TRACER AAV particles comprising a viral genome with a nucleic acid sequence encoding one or more siRNA molecules may be used as a therapy for Amyotrophic Lateral Sclerosis.

In some embodiments, the TRACER AAV particles comprising a viral genome with a nucleic acid sequence encoding one or more siRNA molecules may be used as a therapy for Huntington's Disease.

In some embodiments, the TRACER AAV particles comprising a viral genome with a nucleic acid sequence encoding one or more siRNA molecules may be used as a therapy for Parkinson's Disease.

In some embodiments, the TRACER AAV particles comprising a viral genome with a nucleic acid sequence encoding one or more siRNA molecules may be used as a therapy for Friedreich's Ataxia.

In some embodiments, the administration of TRACER AAV particles comprising a viral genome with a nucleic acid sequence encoding one or more siRNA molecules to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the protein levels of a target protein by at least 50%. As a non-limiting example, the TRACER AAV particles may lower the proteins levels of a target protein by at least 40%.

In some embodiments, the administration of TRACER AAV particles comprising a viral genome with a nucleic acid sequence encoding one or more siRNA molecules to a subject may lower the expression of a target protein in a subject. The expression of a target protein may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the expression of a target protein by at least 50%. As a non-limiting example, the TRACER AAV particles may lower the expression of a target protein by at least 40%.

In some embodiments, the administration of TRACER AAV particles comprising a viral genome with a nucleic acid sequence encoding one or more siRNA molecules to a subject may lower the expression of a target protein in the CNS of a subject. The expression of a target protein may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the TRACER AAV particles may lower the expression of a target protein by at least 50%. As a non-limiting example, the TRACER AAV particles may lower the expression of a target protein by at least 40%.

In some embodiments, the TRACER AAV particles comprising a viral genome with a nucleic acid sequence encoding one or more siRNA molecules may be used to suppress a target protein in astrocytes in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in astrocytes may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress a target protein in microglia. The suppression of the target protein in microglia may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress target protein in cortical neurons. The suppression of a target protein in cortical neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress a target protein in hippocampal neurons. The suppression of a target protein in the hippocampal neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90°/%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress a target protein in DRG and/or sympathetic neurons. The suppression of a target protein in the DRG and/or sympathetic neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress a target protein in sensory neurons in order to treat neurological disease. Target protein in sensory neurons may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 2040%, 2045%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in the sensory neurons may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 545%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45°/%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to suppress a target protein and reduce symptoms of neurological disease in a subject. The suppression of target protein and/or the reduction of symptoms of neurological disease may be, independency, reduced or suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 1540%, 1545%, 15-50%, 15-55%, 15-60%, 15-65%, 15-70%, 15-75%, 15-80%, 15-85%, 15-90%, 15-95%, 20-30%, 20-35%, 2040%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 3545%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the TRACER AAV particles comprising a viral genome with a nucleic acid sequence encoding one or more siRNA molecules may be used to reduce the decline of functional capacity and activities of daily living as measured by a standard evaluation system such as, but not limited to, the total functional capacity (TFC) scale.

In some embodiments, the present composition is administered as a solo therapeutic or as combination therapeutic for the treatment of neurological disease.

The TRACER AAV particles comprising a viral genome with a nucleic acid sequence encoding one or more siRNA molecules may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the TRACER AAV particles comprising a viral genome with a nucleic acid sequence encoding one or more siRNA molecules can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

Compounds tested for treating neurological disease which may be used in combination with the TRACER AAV particles comprising a viral genome with a nucleic acid sequence encoding one or more siRNA molecules include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the TRACER AAV particles comprising a viral genome with a nucleic acid sequence encoding one or more siRNA molecules for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF. Colivelin, Xaliproden, Thyrotrophin-releasing hormone and ADNF, and variants thereof.

In one aspect, the TRACER AAV particle encoding the nucleic acid sequence for the at least one siRNA duplex targeting the gene of interest may be co-administered with TRACER AAV particles expressing neurotrophic factors such as AAV-IGF-I (See e.g., Vincent et al., Neuromolecular medicine, 2004, 6, 79-85: the content of which is incorporated herein by reference in its entirety) and AAV-GDNF (See e.g., Wang et al., J Neurosci., 2002, 22, 6920-6928; the contents of which are incorporated herein by reference in their entirety).

In some embodiments, administration of the TRACER AAV particles to a subject will reduce the expression of a target protein in a subject and the reduction of expression of the target protein will reduce the effects and/or symptoms of neurological disease in a subject.

### Definitions

Adeno-associated virus: As used herein, the term “adeno-associated virus” or “AAV” refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR. As used herein “AAV particles of the disclosure” are AAV particles comprising a parent capsid sequence with at least one targeting peptide insert. AAV particles of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted. In some embodiments, the AAV particle may have a targeting peptide inserted into the capsid to enhance tropism for a desired target tissue. It is to be understood that reference to the AAV particles of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Amplicon: As used herein, “amplicon” may refer to any piece of RNA or DNA formed as the product of amplification events. e.g. PCR. In some embodiments, full-length capsid amplicons may be used as templates for next generation sequencing (NGS) library generation. Full-length capsid amplicons may be used for cloning into a DNA library for any number of additional rounds of AAV selection as described herein.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biopanning: As used herein, the term “biopanning” refers to an AAV capsid library selection process comprising administration of an AAV particle with enhanced tissue- and/or cell type-specific transduction to a cell and/or subject; extraction of nucleotides encoded by said AAV particle from said transduced tissue- and/or cell type-specific; and, use of the extracted nucleotides for cloning into a nucleotide library for the generation of AAV particles for subsequent rounds of the same.

Capsid: As used herein, the term “capsid” refers to the protein shell of a virus particle.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Element: As used herein, the term “element” refers to a distinct portion of an entity. In some embodiments, an element may be a polynucleotide sequence with a specific purpose, incorporated into a longer polynucleotide sequence.

Encapsulate: As used herein, the term “encapsulate” means to enclose, surround or encase. As an example, a capsid protein often encapsulates a viral genome.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle (active ingredient) and an excipient, and/or an inactive ingredient.

Fragment: A “fragment,” as used herein, refers to a portion. For example, an antibody fragment may comprise a CDR, or a heavy chain variable region, or a scFv, etc.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation. e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Insert: As used herein the term “insert” may refer to the addition of a targeting peptide sequence to a parent AAV capsid sequence. An “insertion” may result in the replacement of one or more amino acids of the parent AAV capsid sequence. Alternatively, an insertion may result in no changes to the parent AAV capsid sequence beyond the addition of the targeting peptide sequence.

Inverted terminal repeat: As used herein, the term “inverted terminal repeat” or “ITR” refers to a cis-regulatory element for the packaging of polynucleotide sequences into viral capsids.

Library: As used herein, the term “library” refers to a diverse collection of linear polypeptides, polynucleotides, viral particles, or viral vectors. As examples, a library may be a DNA library or an AAV capsid library.

Neurological disease: As used herein, a “neurological disease” is any disease associated with the central or peripheral nervous system and components thereof (e.g., neurons).

Naturally Occurring: As used herein, “naturally occurring” or “wild-type” means existing in nature without artificial aid, or involvement of the hand of man.

Orthogonal evolution: As used herein, the term “orthogonal evolution” refers to a method wherein AAV particles are administered for a first round of AAV selection as described herein across a set of any number of cell- and/or subject-types that may be from different species and/or strains, and wherein any number of additional, i.e., subsequent, AAV selection rounds are performed either across a set of any number of cell- and/or subject-types that may be from different species and/or strains, or across a set of any number of cell- and/or subject-types that may be from the same species and/or strain.

Open reading frame: As used herein, “open reading frame” or “ORF” refers to a sequence which does not contain a stop codon in a given reading frame.

Parent sequence: As used herein, a “parent sequence” is a nucleic acid or amino acid sequence from which a variant is derived. In some embodiments, a parent sequence is a sequence into which a heterologous sequence is inserted. In other words, a parent sequence may be considered an acceptor or recipient sequence. In some embodiments, a parent sequence is an AAV capsid sequence into which a targeting sequence is inserted.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein. “prophylactic” refers to a therapeutic or course of action used to prevent the spread of disease.

Prophylaxis: As used herein, a “prophylaxis” refers to a measure taken to maintain health and prevent the spread of disease.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

Sample: As used herein, the term “sample” or “biological sample” refers to a subset of its tissues, cells or component parts (e.g. body fluids, including but not limited to blood, serum, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a self-complementary viral genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Targeting peptide: As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism. It is to be understood that a targeting peptide is encoded by a targeting polynucleotide which may similarly be inserted into a parent polynucleotide sequence. Therefore, a “targeting sequence” refers to a peptide or polynucleotide sequence for insertion into an appropriate parent sequence (amino acid or polynucleotide, respectively).

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral Genome: As used herein, the terms “viral genome” or “vector genome” refer to the nucleic acid sequence(s) encapsulated in an AAV particle. A viral genome comprises a nucleic acid sequence with at least one payload region encoding a payload and at least one ITR.

### EQUIVALENTS AND SCONE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. TRACER Proof of Concept

Proof-of-concept experiments were conducted by placing the genes encoding an AAV9 peptide display capsid library under the control of either the neuron-specific synapsin promoter (SYN or Syn) or the astrocyte-specific GFAP promoter. Following intravenous administration to C57BL/6 mice, RNA was recovered from brain tissue and used for further library evolution. Next-generation sequencing (NGS) showed sequence convergence between animals after only two rounds of selection. Interestingly, several variants highly similar to the PHP.eB capsid were recovered, suggesting that our method allowed a rapid selection of high-performance capsids. A subset of capsids having peptide sequences with high CNS enrichment was selected for further study. It is understood that any promoter may be selected depending on the desired tropism. Examples of such promoters are found in Table 2.

TABLE 2
Promoters, tissue and cell type

| Promoter same | Tissue | Cell type |
|---|---|---|
| B29 promoter | Blood | B cells |
| Immunoglobulin heavy | Blood | B cells |
| chain promoter | | |
| CD45 promoter | Blood | Hematopoietic |
| Mouse INF-β promoter | Blood | Hematopoietic |
| CD45 SV40/CD45 promoter | Blood | Hematopoietic |
| WASP promoter | Blood | Hematopoietic |
| CD43 promoter | Blood | Leuko & Platelets |
| CD43 SV40/CD43 promoter | Blood | Leuko & Platelets |
| CD68 promoter | Blood | Macrophages |
| GPIIb promoter | Blood | Megakaryocyte |
| CD14 promoter | Blood | Monocytes |
| CD2 promoter | Blood | T cells |
| Osteocalcin | Bone | Osteoblasts |
| Bone sialoprotein | Bone | Osteoblasts |
| OG-2 promoter | Bone | Osteoblasts, odontoblasts |
| GFAP promoter | Brain | Astrocytes |
| Vga | Brain | GABAergic neurons |
| Vglut2 | Brain | glutamatergic neurons |
| NSE/RU5′ promoter | Brain | Neurons |
| SYN1 promoter | Brain | Neurons |
| Neurofilament light chain | Brain | Neurons |
| VGF | Brain | Neurons |
| Nestin | Brain | NSC |
| Chx10 | Eye | All retinal neurons |
| PrP | Eye | All retinal neurons |
| Dkk3 | Eye | All retinal neurons |
| Math5 | Eye | Amacrine and horizontal |
| | | cells |
| Ptf1a | Eye | Amacrine and horizontal |
| | | cells |
| Pcp2 | Eye | Bipolar cells |
| Nefh | Eye | Ganglion cells |
| gamma-synuclein gene | Eye | ganglion cells |
| (SNCG) | | |
| Grik4 | Eye | GC |
| Pdgfra | Eye | GC and ONL Müller cells |
| Chat | Eye | GC/Amacrine cells |
| Thy 1.2 | Eye | GC/neural retina |
| hVmd2 | Eye | INL Müller cells |
| Thy 1 | Eye | INL Müller cells |
| Modified αA-crystallin | Eye | Lens/neural retina |
| hRgp | Eye | M- and S-cone |
| mMo | Eye | M-cone |
| Opn4 | Eye | Melanopsin-expressing GC |
| RLBP1 | Eye | Muller cells |
| Glast | Eye | Müller cells |
| Foxg1 | Eye | Müller cells |
| hVmd2 | Eye | Müller cells/optic nerve/INL |
| Trp1 | Eye | Neural retina |
| Six | Eye | Neural retina |
| cx36 | Eye | Neurons |
| Grm6-SV40 eukaryotic | Eye | ON bipolar |
| promoter | | |
| hVmd2 | Eye | Optic nerve |
| Dct | Eye | Pigmented cells |
| Rpc65 | Eye | Retinal pigment epithelium |
| mRho | Eye | Rod |
| Irbp | Eye | Rod |
| bRho | Eye | Rod |
| Pcp2 | Eye | Rod bipolar cells |
| Rhodopsin | Eye | Rod Photoreceptors |
| mSo | Eye | S-cone |
| MLC2v promoter | Heart | Cardiomyocyte |
| aMHC promoter | Heart | Cardiomyocyte |
| rat troponin T (Tnnt2) | Heart | Cardiomyocyte |
| Tie2 | Heart | Endothelial |
| Tcf21 | Heart | Fibroblasts |
| ECAD | Kidney | Collecting duct |
| NKCC2 | Kidney | Loop of Henle |
| KSPC | Kidney | Nephron |
| NPHS1 | Kidney | Podocyte |
| SGLT2 | Kidney | Proximal tubular cells |
| SV40/bAlb promoter | Liver | hepatocytes |
| SV40/hAlb promoter | Liver | hepatocytes |
| Hepatitis B virus core | Liver | hepatocytes |
| promoter | | |
| Alpha fetoprotein | Liver | hepatocytes |
| Surfactant protein B promoter | Lung | AT II cells and Clara cells |
| Surfactant protein C promoter | Lung | AT II cells and Clara cells |
| Desmin | Muscle | Muscle stem cells + |
| | | Myocytes |
| Mb promoter | Muscle | Myocyte |
| Myosin | Muscle | Myocyte |
| Dystrophin | Muscle | Myocyte |
| dMCK and tMCK | Muscle | Myocytes |
| Elastase-1 promoter | Pancreas | Acinar cells |
| PDX1 promoter | Pancreas | Beta cells |
| Insulin promoter | Pancreas | langherans |
| Slco1c1 | Vasculature | BBB Endothelial |
| tie | Vasculature | Endothelial |
| cadherin | Vasculature | Endothelial |
| ICAM-2 | Vasculature | Endothelial |
| claudin 1 | Vasculature | Endothelial |
| Cldn5 | Vasculature | Endothelial |
| Flt-1 promoter | Vasculature | Endothelial |
| Endoglin promoter | Vasculature | Endothelial |

Capsid pools were injected into three rodent species, followed by RNA enrichment analysis for characterization of transduction efficiency in neurons or astrocytes and cross-species performance. Top-ranking capsids were then individually tested and several variants showed CNS transduction similar to or higher than the PHP.eB benchmark. These results suggest that the TRACER platform allows rapid in vivo evolution of AAV capsids in non-transgenic animals with a high degree of tropism improvement. The following examples illustrate the findings in more detail.

### Example 2. Generation of an AAV Vectors Capable of Capsid mRNA Expression in the Absence of Helper Virus

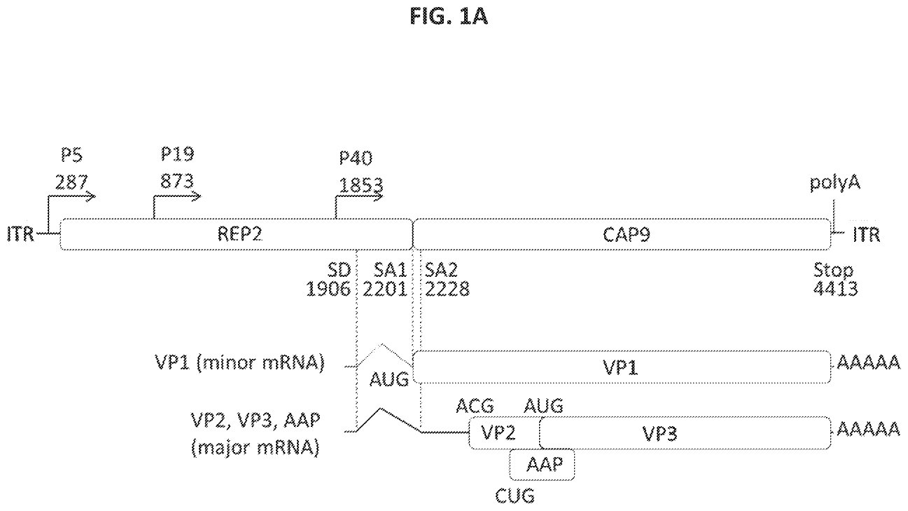

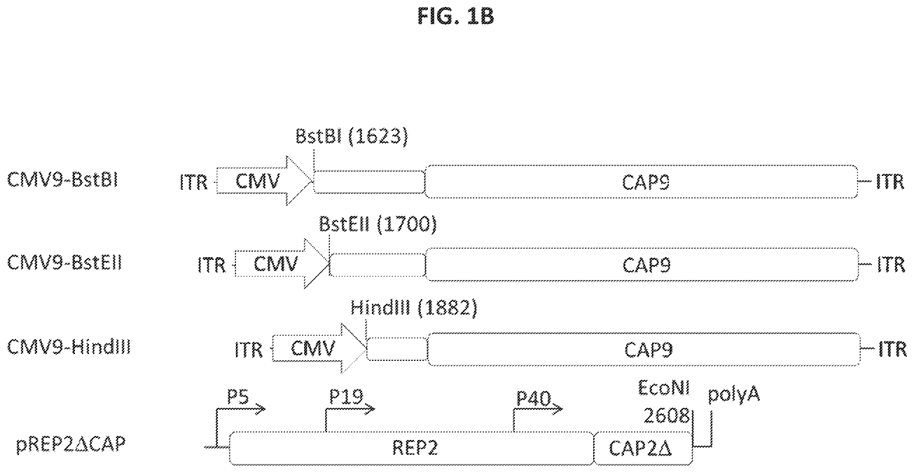

In order to perform cell type- and transduction-restricted in vivo evolution of AAV capsid libraries, a capsid library system was engineered in which the capsid mutant gene can be transcribed in the absence of a helper virus, in a specific cell type. In the wild-type AAV virus, the mRNA encoding the capsid proteins VP1, VP2 and VP3, as well as the AAP accessory protein, are expressed by the P40 promoter located in the 3′ region of the REP gene (FIG. 1A), that is only active in the presence of the REP protein as well as the helper virus functions (Berns et al., 1996, the contents of which are incorporated herein by reference in their entirety). In order to allow expression of the capsid mRNA in animal tissue or in cultured cells, another promoter must be inserted upstream or downstream of the CAP gene. Because of the limited packaging capacity of the AAV capsid, a portion of the REP gene must be deleted to accommodate the extra promoter insertion, and the REP gene must be provided in trans by another plasmid to allow virus production. The minimal viral sequence required for high titer AAV production was determined by introducing a CMV promoter at various locations upstream of the CAP gene of AAV9 (FIG. 1B). The REP protein was provided in trans by the pREP2 plasmid obtained by deleting the CAP gene from a REP2-CAP2 packaging vector using EcoNI and ClaI (SEQ ID NO: 4). For small-scale virus production test, HEK-293T cells grown in DMEM supplemented with 5% FBS and 1× pen/strep were plated in 15-cm dishes and co-transfected with 15 ug of pHelper (pFdelta6) plasmid, 10 ug pREP2 plasmid and 1 ug ITR-CMV-CAP plasmid using calcium phosphate transfection. After 72 hours, cells were harvested by scraping, pelleted by a brief centrifugation and suspended in 1 ml of a buffer containing 10 mM Tris and 2 mM MgCl₂. Cells were lysed by addition of triton X-100 to 0.1% final concentration and treated with 50 U of benzonase for 1 hour. Virus from the supernatants was precipitated with 8% polyethylene glycol and 0.5M NaCl, suspended in 1 ml of 10 mM TRIS-2 mM MgCl₂ and combined with the cell lysate. The pooled virus was adjusted to 0.5M NaCl, cleared by centrifugation for 15 minutes at 4,000×g and fractionated on a step iodixanol gradient of 15%, 25%, 40% and 60% for 3 hours at 40,000 rpm (Zolotukhin et al., 1999, the contents of which are incorporated herein by reference in their entirety). The 40% fraction containing the purified AAV particles was harvested and viral titers were measured by real-time PCR using a Taqman primer/probe mix specific for the 3′-end of REP, shared by all the constructs. Virus yields were significantly lower than the fully wild-type ITR-REP2-CAP9-ITR used as a reference (1.7% to 8.8%), but the CMV-BstEII construct allowed the highest yields of all three CMV constructs. The CMV-HindIII construct, in which most of the P40 promoter sequence is deleted, generated the lowest yield (1.7% of wtAAV9), indicating that even the potent CMV promoter cannot replace the P40 promoter without a severe drop in virus yields. Following these observations, the BstEII architecture (SEQ ID NO: 5), which preserves the minimal P40 sequence and the CAP mRNA splice donor, was used in all further experiments.

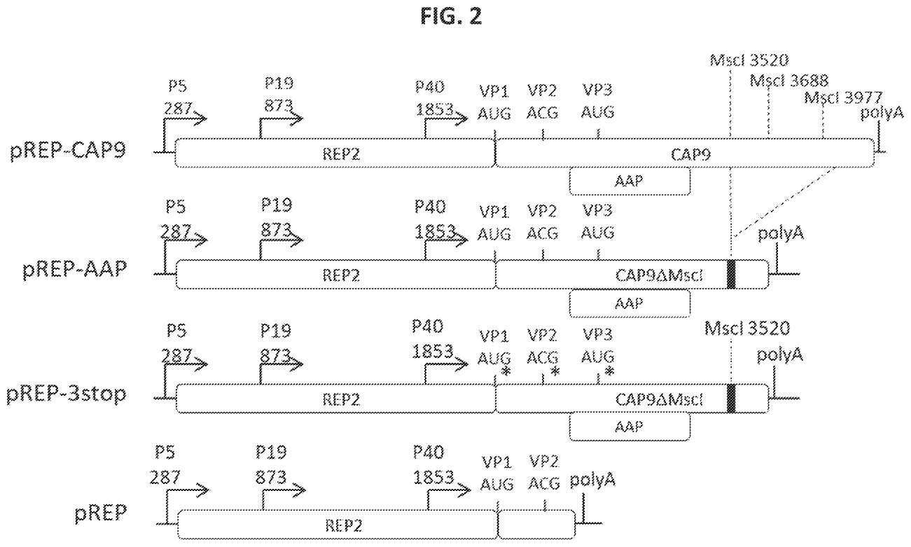

The REP-expressing plasmid was then improved by preserving the AAP reading frame together with a large portion of the capsid gene from the REP2-CAP9 helper vector, which may contain sequences necessary for the regulation of CAP transcription and/or splicing. In order to eliminate the capsid coding potential of the vector, a C-terminus fragment of the capsid gene was deleted by a triple cut with the MscI restriction enzyme followed by self-ligation, in order to obtain the pREP-AAP plasmid (FIG. 2, SEQ ID NO: 6).

An iteration of this construct was engineered by introducing premature stop codons immediately after the start codons of VP1, VP2 and VP3, without perturbing the amino acid sequence of the colinear AAP reading frame (FIG. 2). This construct was named pREP-3stop (SEQ ID NO: 7). A neuron-specific syn-CAP9 vector (SEQ ID NO: 8) was derived from the CMV9-BstEII plasmid by swapping the CMV promoter with the neuron-specific human synapsin 1 promoter.

Production efficiency of this Syn-CAP9 was tested as described previously using pREP, pREP-AAP or pREP-3stop plasmid to supply REP in trans. The REP plasmids harboring a longer capsid sequence as well as AAP increased virus yields by approximately 3-fold compared to the pREP plasmid. Virus titers obtained with the pREP-AAP or pREP-3stop vectors reached ˜30% of wild-type AAV9. An important concern with plasmids harboring long homologous regions is the potential for unwanted recombination with the ITR-CAP vector, that would reconstitute a wild-type ITR-REP-CAP vector and contaminate combinatorial libraries.

To evaluate the risk of wild-type virus reconstitution, viral preparations were subjected to real-time PCR with a Taqman probe located in the N terminus of REP. The percentage of capsids containing a detectable full-length REP was less than 0.03% of wild-type virus, which was even lower than the routinely detected 0.1% illegitimate REP-CAP packaging occurring in most recombinant AAV preparations obtained from 293T cell transfection. Because the premature stop codons of the pREP-3stop vector offer an extra layer of safety against potential reconstitution of wild-type capsids and prevents the translation of truncated capsid proteins, the 3stop plasmid was used for all subsequent studies.

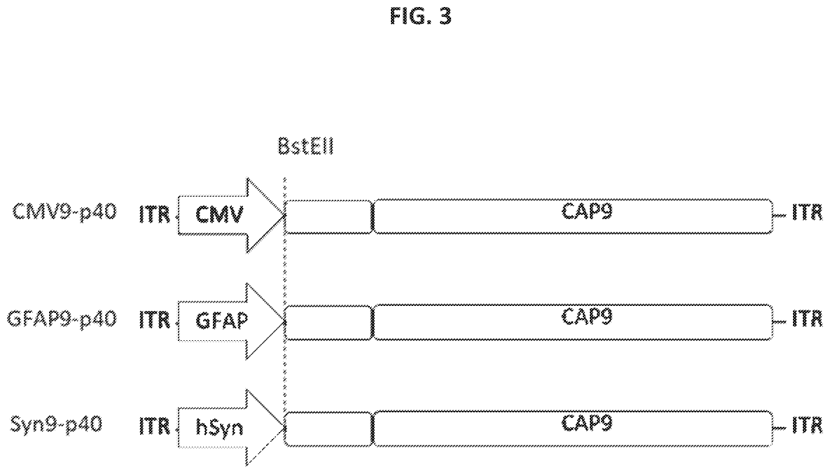

Following this, the feasibility of RNA-driven biopanning in C57BL/6 mice using AAV9-packaged vectors where the AAV9 capsid gene is driven by the CMV promoter, the Synapsin promoter or the astrocyte-specific GFabc1D promoter (SEQ ID NO: 9), hereinafter referred to as GFAP promoter (Brenner et al., 2008, the contents of which are incorporated herein by reference in their entirety) was tested (FIG. 3). The three vectors were produced in HEK-293T cells as previously described and analyzed by PAGE-silver stain. All vectors showed a normal ratio of VP1, VP2 and VP3 capsid proteins, indicating that the promoter architecture does not disrupt the balance of capsid protein expression. Six-week old male C57BL/6 mice were injected intravenously with 1e12 VG per mouse and sacrificed after 28 days. DNA biodistribution and capsid mRNA expression were tested in the brain, liver and heart tissues.

Total DNA was extracted from brain, liver and heart tissues using Qiagen DNeasy Blood and Tissue columns, and viral DNA was quantified by real-time PCR using a Taqman probe located in the VP3 N-terminal region. DNA abundance was normalized using a pre-designed probe detecting the single-copy transferrin receptor gene (Life Technologies ref. 4458366). Viral DNA was highly abundant in the liver and to a lower extent in the heart. The DNA distribution did not show any noticeable difference between the three vectors. RNA was extracted with Qiagen RNeasy plus universal kit following manufacturer's instructions, then treated with ezDNAse (Qiagen) to remove residual DNA, and reverse transcribed with Superscript IV (Life technologies).

RNA expression was evaluated using the same VP3 probe used to quantify viral DNA and normalized using TBP as a reference RNA (Life technologies Mm01277042_m1). In the brain, the GFAP promoter showed the strongest expression level, and the Synapsin (Syn) promoter showed comparable expression to the potent CMV promoter. In the liver, all promoters resulted in a similar expression level, which could be the result of leaky expression at a very high copy number. In the heart, the cell type specificity of the Syn and GFAP promoters was evident, since they allowed only ˜3 and 10% of CMV expression, respectively, despite similar DNA biodistribution.

Overall, the experiment showed that mRNA from transduction-competent capsids could be recovered from various animal organs, including weakly transduced tissues such as the brain.

### Example 3. AAV Vector Configuration

Various vector configurations were explored toward increasing RNA expression to maximize library recovery. The CMV promoter was replaced by a hybrid CMV enhancer/Chicken beta-actin promoter sequence (Niwa et al., 1991, the contents of which are incorporated herein by reference in their entirety) and a potent cytomegalovirus-beta-globin hybrid intron derived from the AAV-MCS cloning vector (Stratagene) was inserted between the promoter sequence and the capsid gene, as introns have been shown to increase mRNA processing and stability (Powell et al., 2015, the contents of which are incorporated herein by reference in their entirety). This resulted in the constructs CAG9 (SEQ ID NO: 10), SYNG9 (SEQ ID NO: 11) and GFAPG (SEQ ID NO: 12).

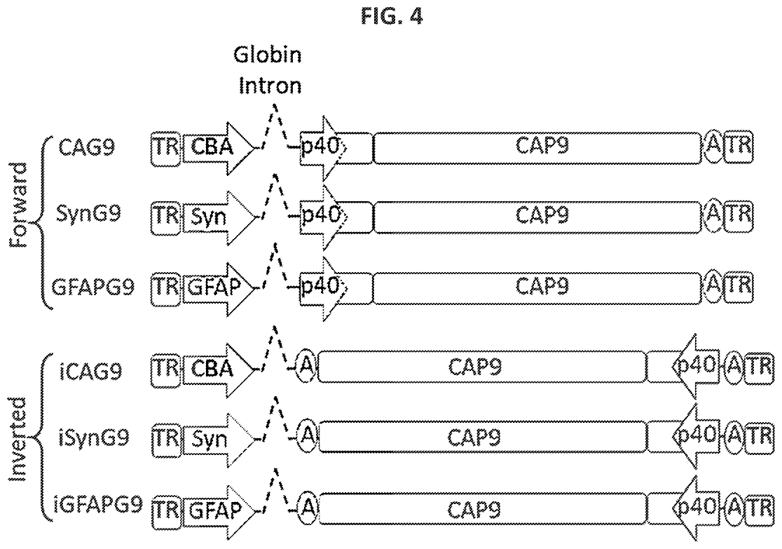

An inverted vector configuration was also tested where the helper-independent promoter was placed downstream of the capsid gene in reverse orientation, in order to avoid potential interference with the P40 promoter (FIG. 4). This configuration allows the expression of an antisense capsid transcript in animal tissue. Because most polyadenylation signals (AATAAA) are orientation-dependent, it was hypothesized that the natural AAV capsid polyA would not prematurely terminate transcription when placed in reverse orientation. All constructs were co-transfected with pHelper and pREP-3stop plasmids to generate AAV9-packaged virions that were used to transduce HEK-293T cells at a MOI of 1e4 VG per cell. RNA was extracted 48 hours post-transfection and reverse transcribed using the Quantitect kit (Qiagen).

PCR was performed with primers allowing amplification of the full-length capsid or a partial sequence localized close to the C-terminus. Overall, the presence of an intron had little influence on the expression from low-activity promoters Syn and GFAP, which indicates that mRNA splicing did not alleviate promoter repression in nonpermissive cells. The combination of the CMV enhancer with a Chicken beta-actin promoter and the hybrid intron allowed a significantly higher (>10-fold) mRNA expression compared to CMV promoter alone.

When comparing endpoint PCR amplification between forward and inverted intronic vectors, a discrepancy was obvious between full-length and partial capsid amplicons, which led us to question the integrity of capsid RNA. When cDNA from inverted iCAG9 genome was amplified using primers flanking the full-length capsid, multiple low-molecular weight bands were detected, whereas the forward orientation vector allowed amplification of a single product with the expected length. Sanger sequencing of low-molecular weight amplicons showed that each band corresponded to an illegitimate splicing product from the antisense capsid RNA.

In light of these results, the forward tandem promoter architecture was used for subsequent experiments.

Splice-specific PCR amplification was tested to avoid amplification of residual DNA present in RNA preparations. Two candidate PCR primers overlapping the CMV/Globin exon-exon junction were designed and tested for amplification of cDNA (spliced) or plasmid DNA (still containing the intron sequence): GloSpliceF4 primer (SEQ ID NO: 26) and GloSpliceF6 primer (SEQ ID NO: 13). The GloSpliceF6 primer (SEQ ID NO: 13) allowed a fully specific amplification from cDNA without producing a detectable amplicon from the plasmid DNA sequence. This primer was used in subsequent assays to ascertain the absence of amplification from contaminating DNA.

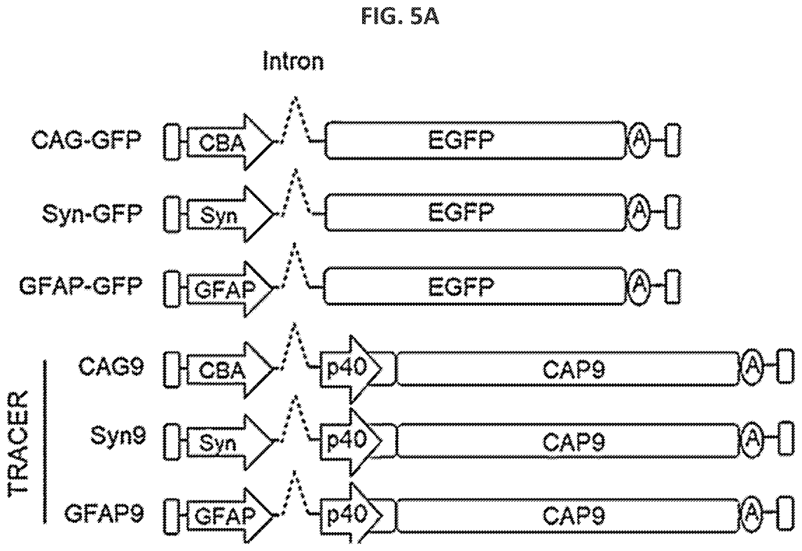

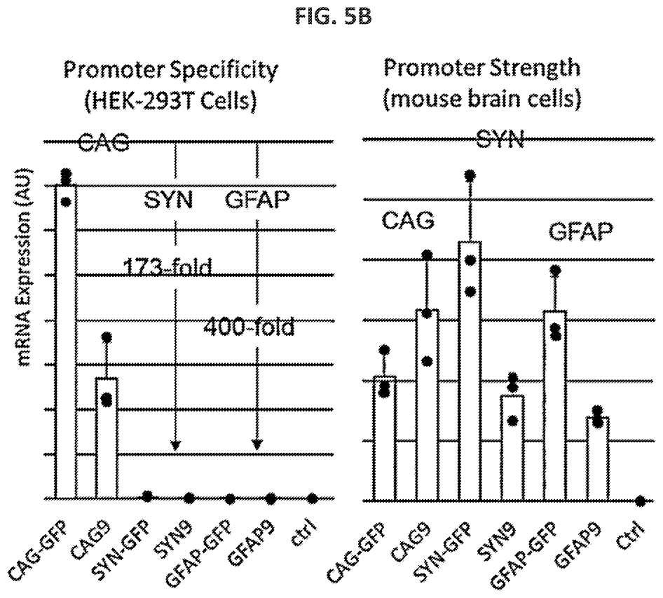

Tandem constructs were then tested for potential interference of the P40 promoter with the cell-specific promoter placed upstream. For this, two series of AAV genomes were tested for transgene mRNA expression in HEK-293T cells. A series of transgenes where the GFP gene was placed immediately downstream of the CAG, SYNG or GFAPG promoter without the P40 sequence were tested, and compared to the library constructs where the AAV9 capsid was placed downstream of the P40 promoter (FIG. 5). All genomes were packaged into the AAV9 capsid and used to infect HEK-293T at a MOI of 1e4 VG per cell. RNA was extracted 48 hours post-infection and transgene RNA was quantified by using a Taqman primer/probe mix specific for the spliced globin exon-exon junction. The expression from the CAG promoter was similar between the GFP and the P40-CAP9 constructs (2-fold lower in p40-CAP9, within the error margin of AAV titration). Expression from the synapsin promoter was drastically lower with both constructs and even lower for GFAP-driven mRNA. This was expected since HEK-293T cells are not permissive to Synapsin or GFAP promoter expression. Overall, this experiment confirmed that the presence of the P40 sequence did not alter the cell type specificity of synapsin or GFAP promoters.

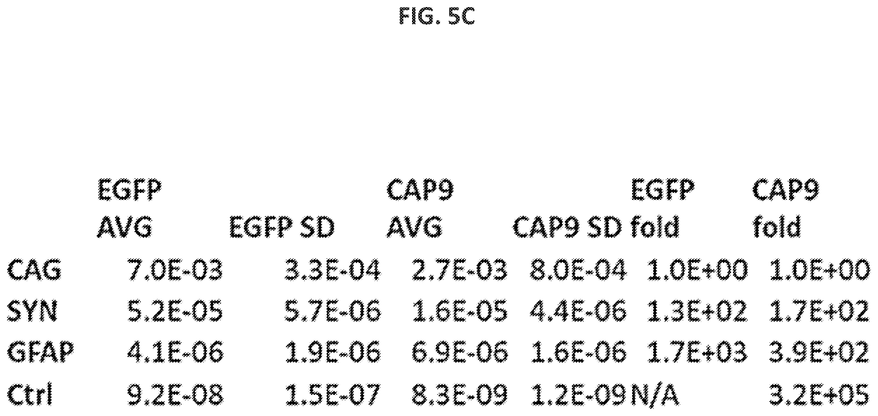

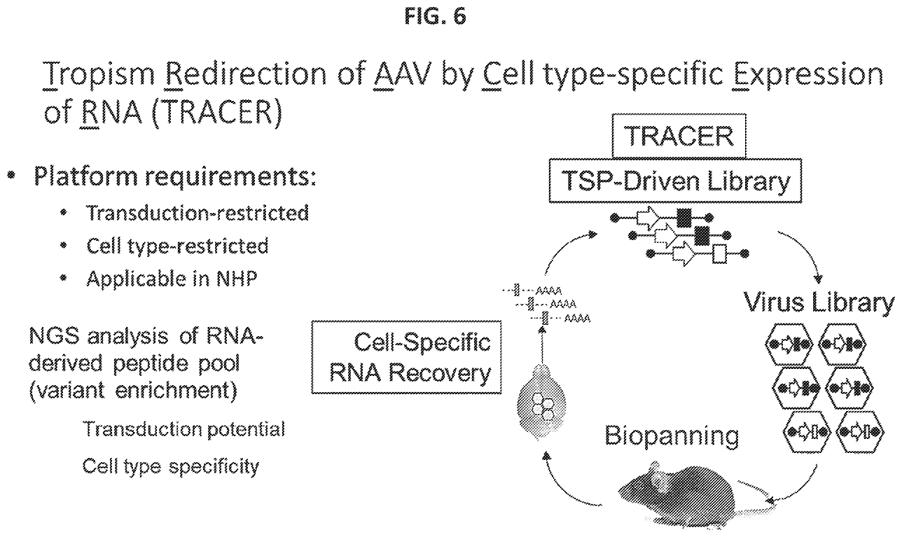

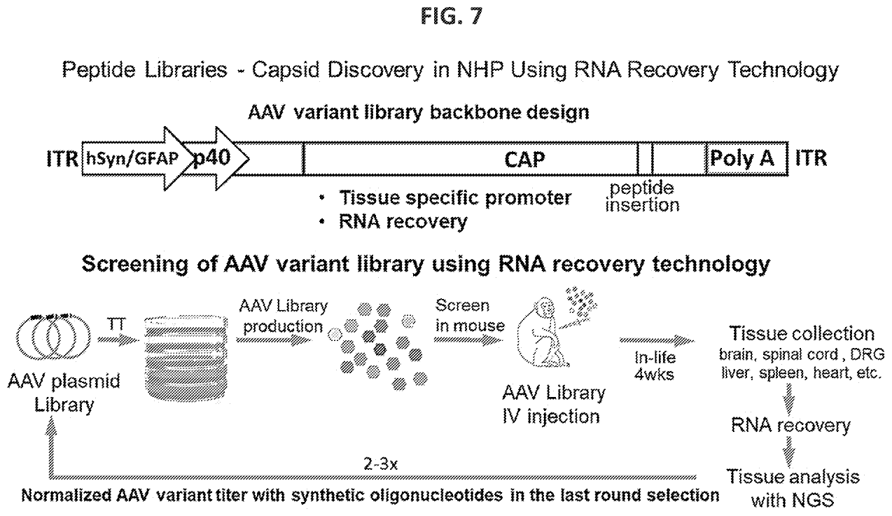

This novel platform was termed TRACER (Tropism Redirection of AAV by Cell type-specific Expression of RNA). The TRACER platform solves the problems of standard methods including transduction and cell-type restrictions (FIG. 6). Use of the TRACER system is well suited to capsid discovery where targeting peptide libraries are utilized. Screening of such a library may be conducted as outlined in FIG. 7.

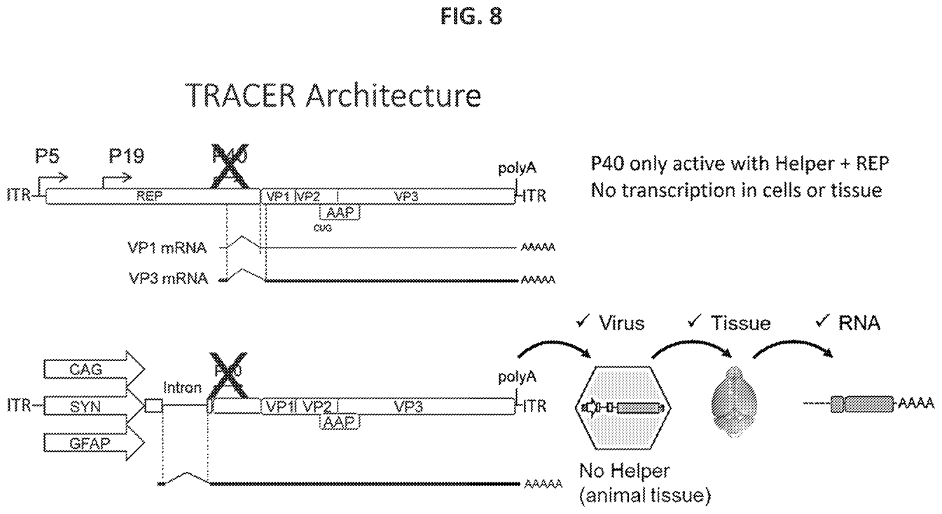

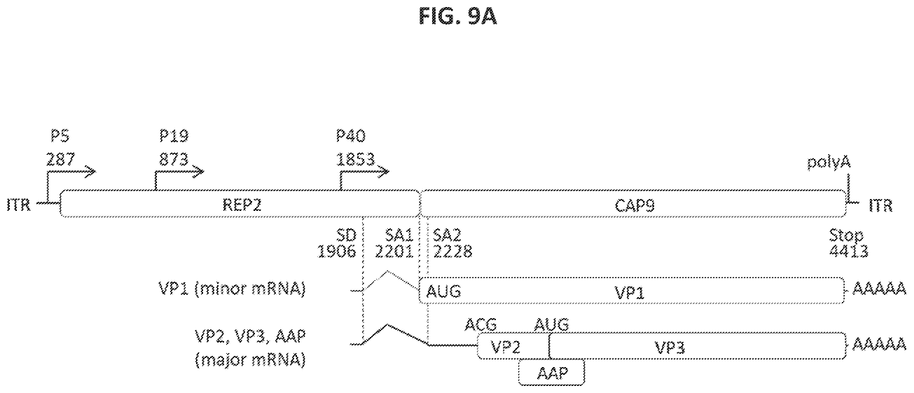

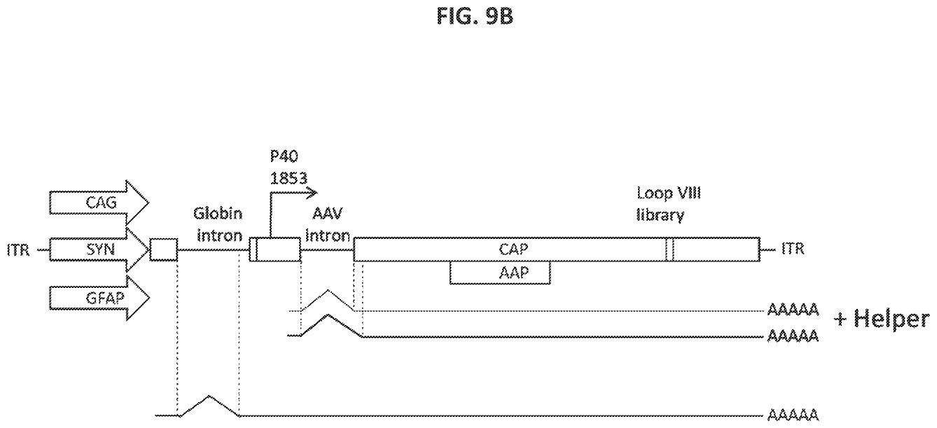

While several variations of the AAV vectors which encode the capsids as payloads are taught herein, one canonical design is shown in FIG. 8 and in FIG. 9A and FIG. 9B.

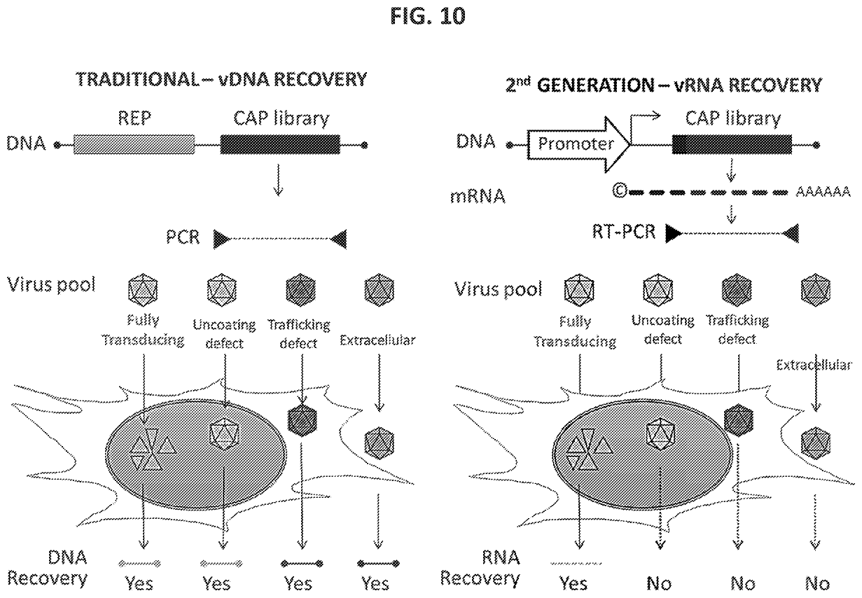

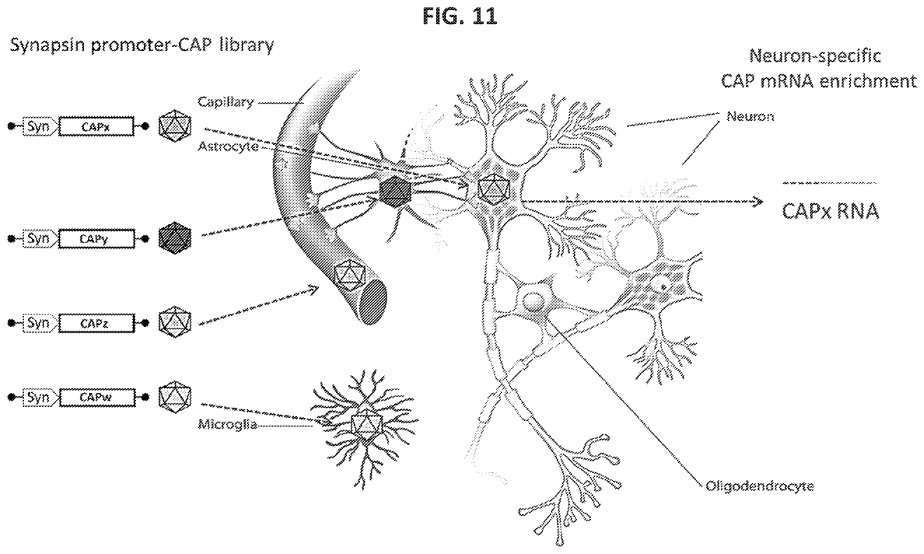

Further advantages of the TRACER platform relate to the nature of the virus pool and the recovery of RNA only from fully transduced cells (FIG. 10). Consequently, capsid discovery can be accelerated in a manner that results in cell and/or tissue specific tropism (FIG. 11).

### Example 4. Generation of Peptide Display Libraries and Cloning-Free Amplification

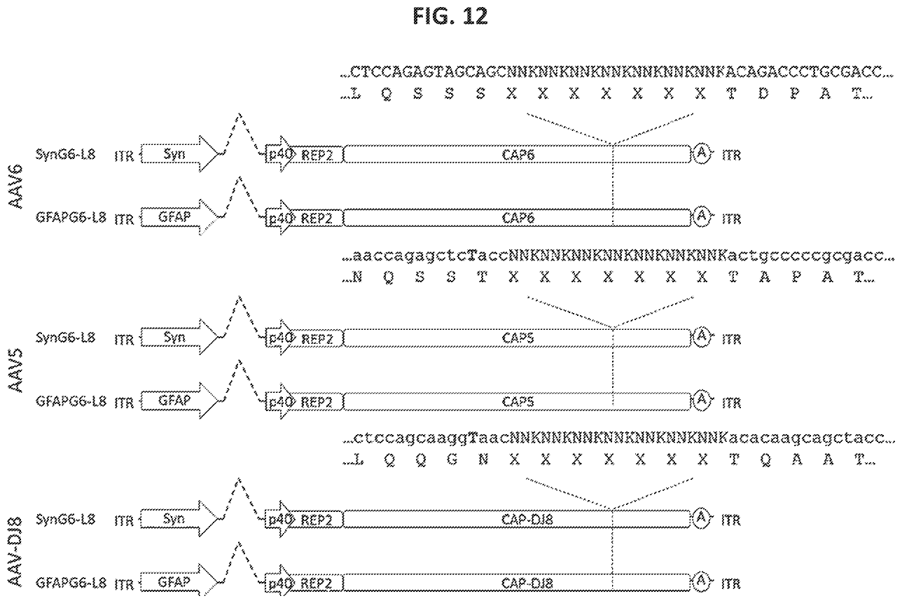

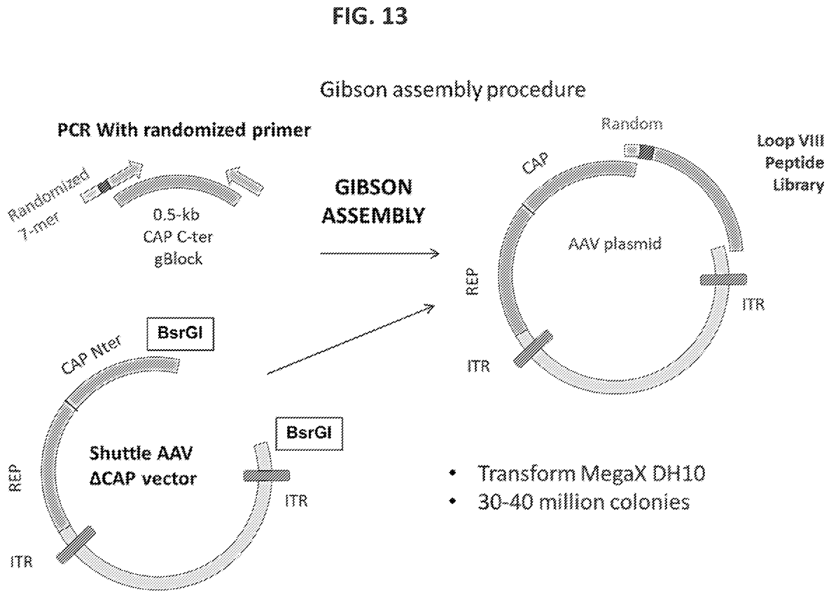

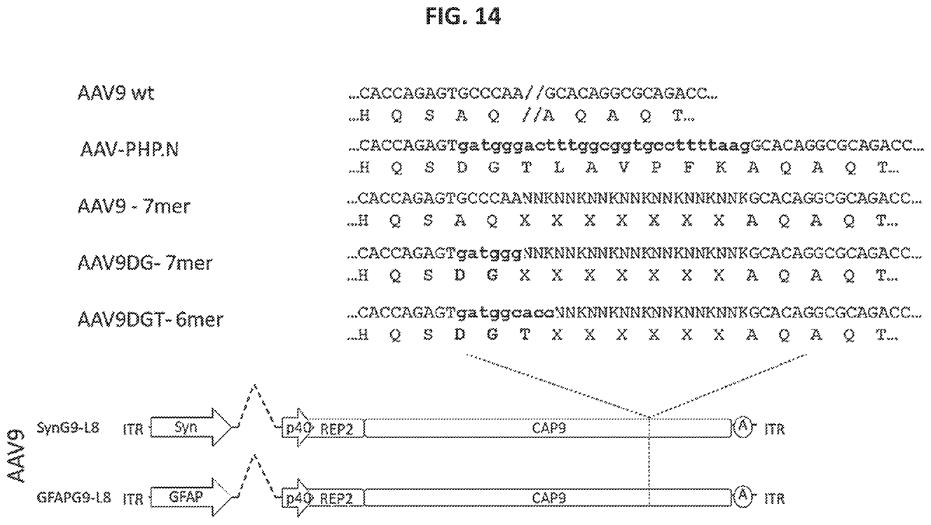

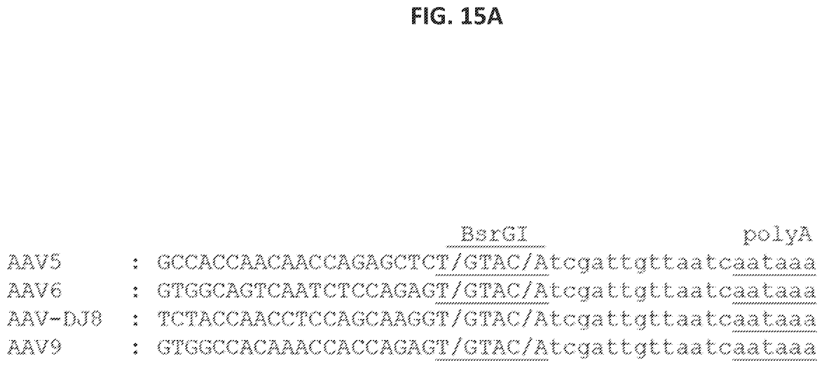

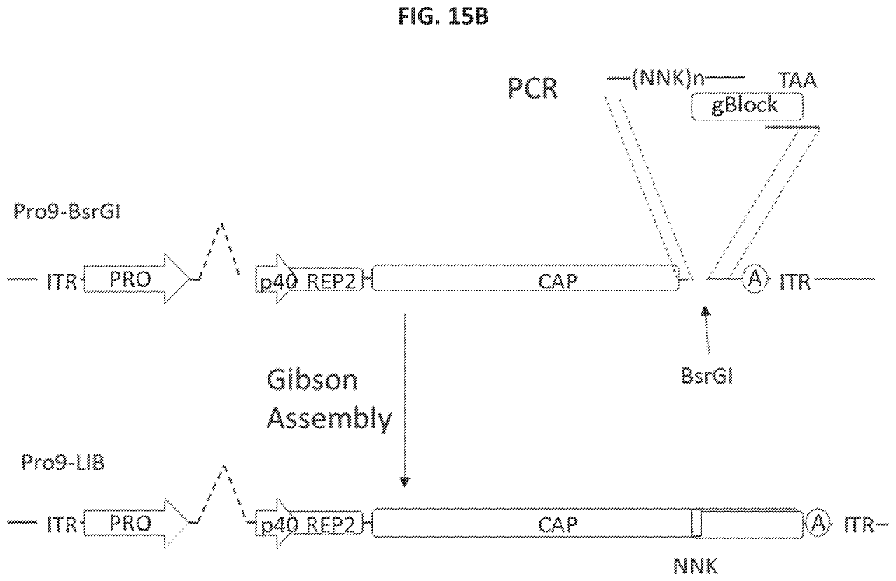

Several peptide display capsid libraries were generated by insertion of seven contiguous randomized amino acids into the surface-exposed hypervariable loop VIII region of AAV5, AAV6, or AAV-DJ8 capsids (FIG. 12 and FIG. 13) as well as AAV9 (FIG. 14). For AAV9 libraries, two extra libraries by modifying residues at positions −2, −1 and +1 of the insertion to match the flanking sequence of the highly neurotrophic PHP.eB vector (Chan et al., 2018, the contents of which are incorporated herein by reference in their entirety). In order to facilitate the insertion of various loops and to prevent contamination by wild-type capsids, defective shuttle vectors were generated in which the C-terminal region of the capsid gene comprised between the loop VIII and the stop codon was deleted and replaced by a unique BsrGI restriction site (FIG. 15A, B). Degenerate primers containing randomized NNK (K=T or G) sequences able to encode all amino acids were synthesized by IDT and used to amplify the missing capsid fragment using gBlock (IDT) double-stranded linear DNA as templates (SEQ ID NO 14, 15, 16, 17). Linear PCR templates were preferred to plasmids in order to completely prevent the possibility of plasmid carryover in the PCR reaction. Amplicons containing the random library sequence (500 ng) were inserted in the shuttle plasmid linearized by BsrGI (2 ug) using 100 ul of NEBuilder HiFi DNA assembly master mix (NEB) during 30 minutes at 50° C. Unassembled linear templates were eliminated by addition of 5 ul of T5 exonuclease to the reaction and digestion for 30 minutes at 37° C. The entire reaction was purified with DNA Clean and Concentrator-5 and quantified with a nanodrop to estimate the efficiency of assembly. This method routinely allows the recovery of 0.5-1 ug assembled material.

gBlock templates were engineered by introducing silent mutations to remove unique restriction sites, to allow selective elimination of wild-type virus contaminants from the libraries by restriction enzyme treatment. As an example, AAV9 gBlock was engineered to remove BamHI and AfeI sites present in the parental sequence (SEQ ID NO 17).

### Example 5. Cloning Free Amplification

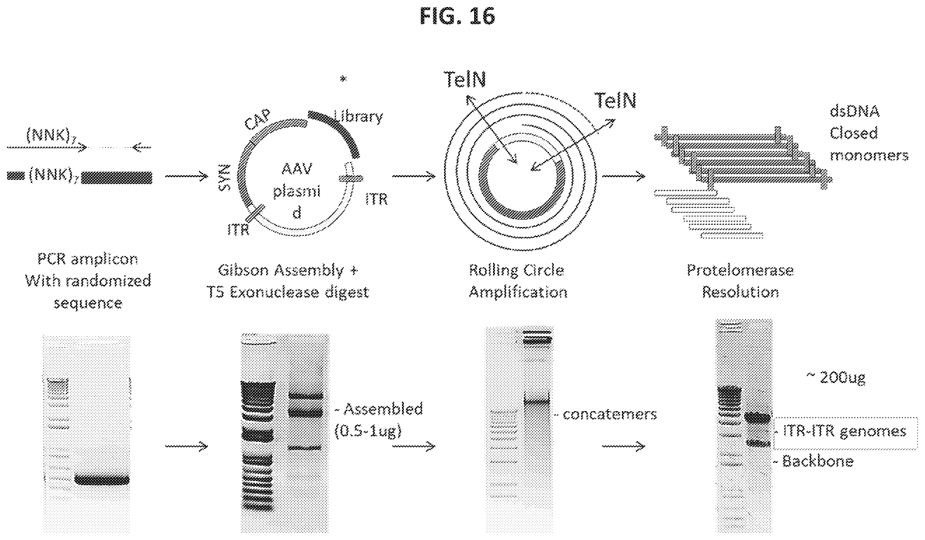

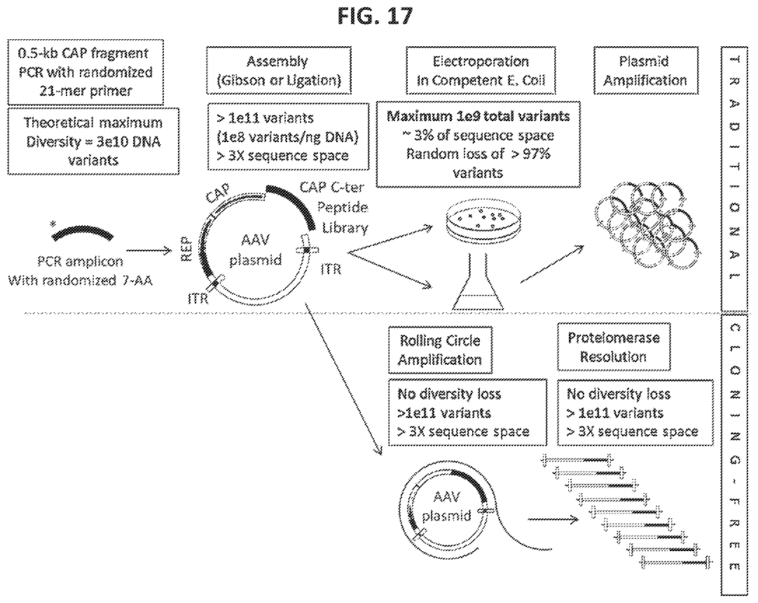

Transformation of assembled library DNA into competent bacteria represents a major bottleneck in library diversity, since even highly competent strains rarely exceed 1e7-1e8 colonies per transformation. By comparison, 100 nanograms of a 6-kilobase plasmid contain 1.5e10 DNA molecules. Therefore, bacterial transformation arbitrarily eliminates more than 99% of DNA species in a given pool. A cloning-free method was therefore created that allows >100-fold amplification of Gibson-assembled DNA while bypassing the bacterial transformation bottleneck (FIG. 16 and FIG. 17). A protocol based on rolling-circle amplification was optimized, which allows unbiased exponential amplification of circular DNA templates with an extremely low error rate (Hutchinson et al., 2005, the contents of which are incorporated herein by reference in their entirety). One issue with rolling circle amplification is that it produces very large (˜70 kilobases on average) heavily branched concatemers that must be cleaved into monomers for efficient cell transfection. This process can be accomplished by several methods, for example by using restriction enzymes to generate open-ended linear templates (Hutchinson et al., 2005, Huovinen, 2012, the contents of each of which are incorporated herein by reference in their entirety), or CRE-Lox recombination to generate self-ligated circular templates (Huovinen et al., 2011, the contents of which are incorporated herein by reference in their entirety). However, open-ended DNA is sensitive to degradation by cytoplasmic exonucleases, and the CRE recombination method showed relatively low efficiency (our unpublished observations). Therefore, an alternative monomer resolution method was chosen based on the use of TelN protelomerase (Rybchin et al., 1999, the contents of which are incorporated herein by reference in their entirety), an enzyme that catalyzes the formation of closed-ended linear “dogbone” DNA monomers that are highly suitable for mammalian cell transfection (Heinrich et al., 2002, the contents of which are incorporated herein by reference in their entirety).

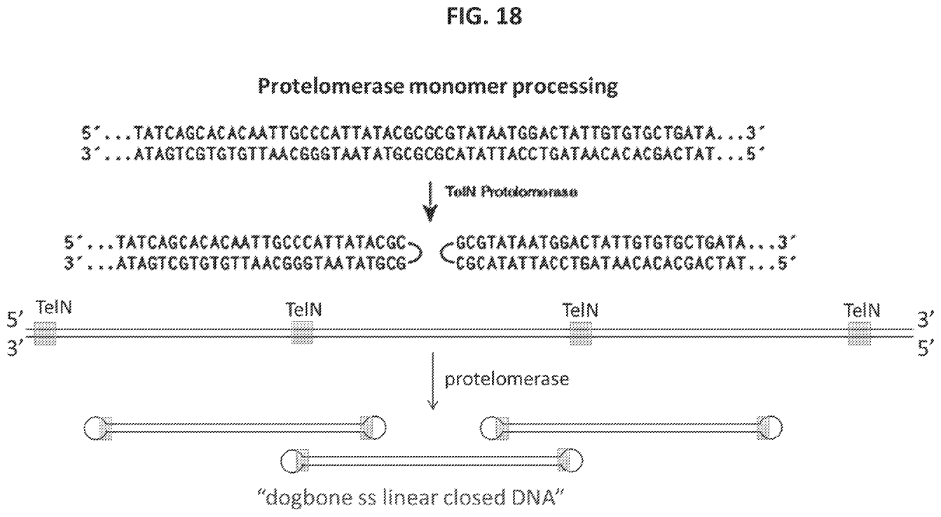

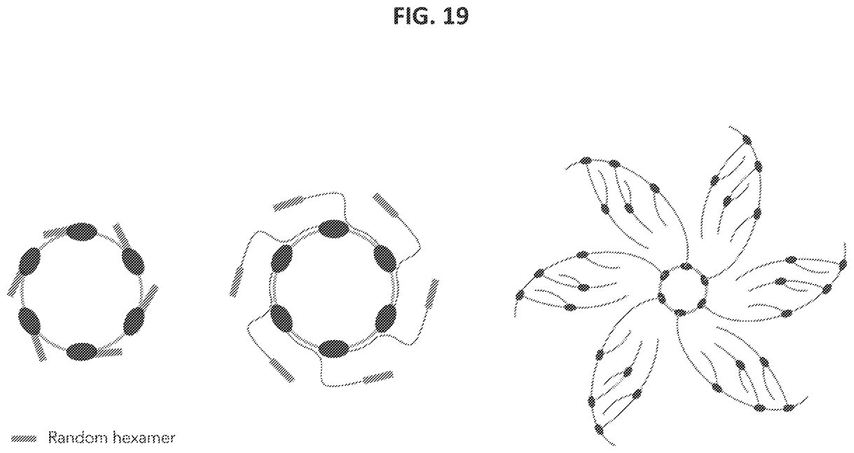

To that end, a protelomerase recognition sequence (SEQ ID NO: 59) was introduced outside both ITRs in all the BsrGI shuttle vectors used for capsid library insertion (the asterisk depicts the position were the two complementary strands are covalently linked to each other), in order to obtain the following plasmids: TelN-Syn9-BsrGI (SEQ ID NO: 18), TelN-GFAP9-BsrGI (SEQ ID: NO 19), TelN-Syn5-BsrGI (SEQ ID NO: 20), TelN-GFAP5-BsrGI (SEQ ID NO: 21), TelN-Syn6-BsrGI (SEQ ID NO: 22), TelN-GFAP6-BsrGI (SEQ ID NO: 23), TelN-SynDJ8-BsrGI (SEQ ID: NO 24), TelN-GFAPDJ8-BsrGI (SEQ ID NO: 25) (FIG. 18). Several methods for rolling circle amplification were tested, and the best results (high yield and low non-specific amplification) were obtained with the TruePrime technology (Expedeon), which relies on primerless amplification (Picher et al., 2016, the contents of which are incorporated herein by reference in their entirety). An exemplary diagram of cloning-free DNA amplification by rolling circle amplification is presented in FIG. 19.

Briefly, the entire column-purified assembly reaction was used in a 900-ul TruePrime reaction following the manufacturer's instructions and incubated overnight at 30° C. The following day, the rolling circle reaction product was incubated 10 minutes at 65° C. to inactivate the enzymes and was diluted 5-fold in 1× thermoPol buffer with 50 ul protelomerase (NEB) in a 4.5-ml reaction. After 1 hour at 30° C., the reaction was heat-treated for 10 minutes at 70° C. to inactivate the protelomerase, and a 4.5-ul aliquot was run on an agarose gel. The entire reaction was then purified on multiple (10-12) Qiagen QiaPrep 2.0 columns following manufacturer's instructions. The typical yield obtained with this method was 160-180 ug DNA, which indicates >100-fold amplification of the starting material (typically 0.5-1 ug) and provides enough DNA for transfection of 200 cell culture dishes (FIG. 16 and FIG. 17).

The composition of all libraries was tested by next-gen sequencing (NGS) with an Illumina NextSeq sequencing platform to estimate the number of variants and the eventual contamination by wild-type viruses. Amplicons were generated by PCR with Q5 polymerase (NEB) using primers containing Illumina TruSeq adapters and index barcodes. Amplicons were obtained by low-cycle PCR amplification (15 cycles), ran on 3% agarose gels and purified using Zymo gel extraction reagents. Libraries were quantified using a nanodrop, pooled into equimolar mixes and re-quantified with a KAPA library quantification kit following manufacturer's instruction. Libraries were mixed with 20-40% of PhiX control library to increase sequence diversity.

All DNA libraries generated by rolling circle showed a high sequence diversity (typically >1e8 unique variants, beyond the limits of NextSeq sequencing).

### Example 6. Prevention and/or Reduction of Contamination

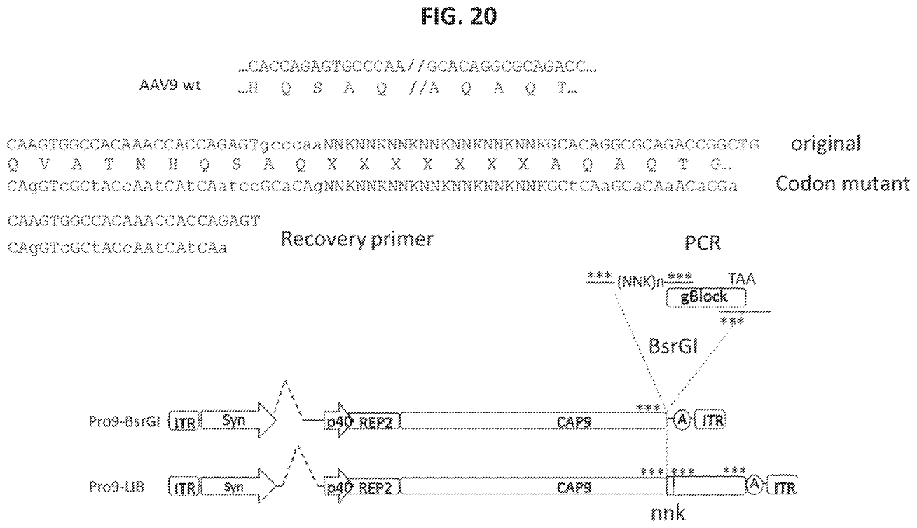

A primer/vector system aimed at completely preventing contamination of AAV9 libraries by wild-type virus, possibly recovered from environmental contamination or from naturally infected primate animal tissues, was created. This was achieved by introducing a maximum number of silent mutations in the sequences surrounding the library insertion site, as well as the sequence immediately before the CAP stop codon, used for PCR amplification (FIG. 20). These libraries showed an extremely low number of wild-type AAV9 detection by NGS (<2 AAV9 reads per 5e7 total reads), suggesting that the alteration of codons surrounding the library amplification and cloning sites is a very efficient way to preserve libraries from environmental or experimental contaminations.

Libraries were produced as described previously by calcium phosphate transfection of HEK-293T cells, dual iodixanol gradient fractionation and membrane ultrafiltration using 100,000 Da MWCO Amicon-15 membranes (Millipore), quantified by real-time PCR and an aliquot was used for NGS amplicon generation and NextSeq sequencing. The diversity of viral libraries was significantly lower than that of DNA libraries (typically ˜1-2e7 unique variants) and showed a very strong counter-selection of variants containing stop codons (from 20% in DNA libraries to ˜1% in virus libraries), evincing a very high rate of cis-packaging, as observed in previous studies (Nonnenmacher et al., 2015, the contents of which are incorporated herein by reference in their entirety).

### Example 7. In Vivo Selection of AAV9 Libraries for Mouse Brain Transduction

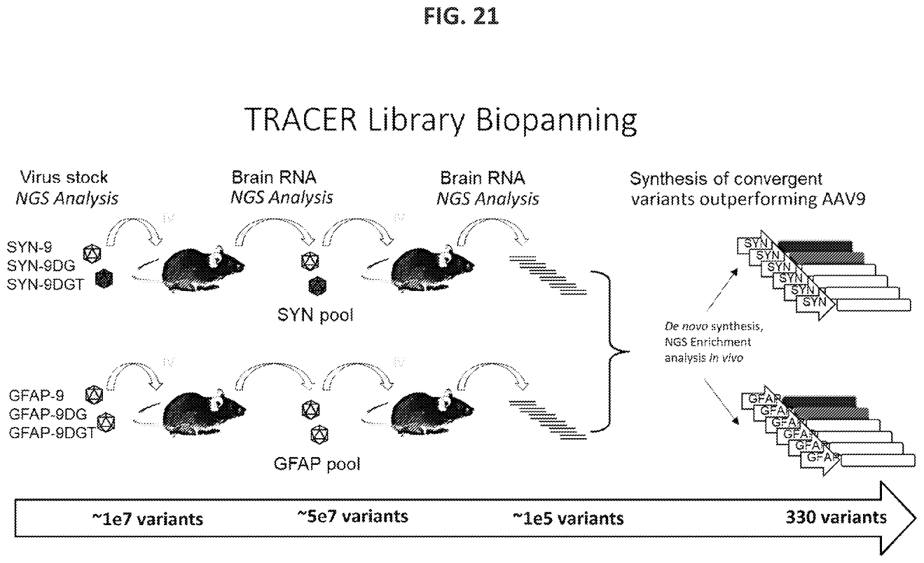

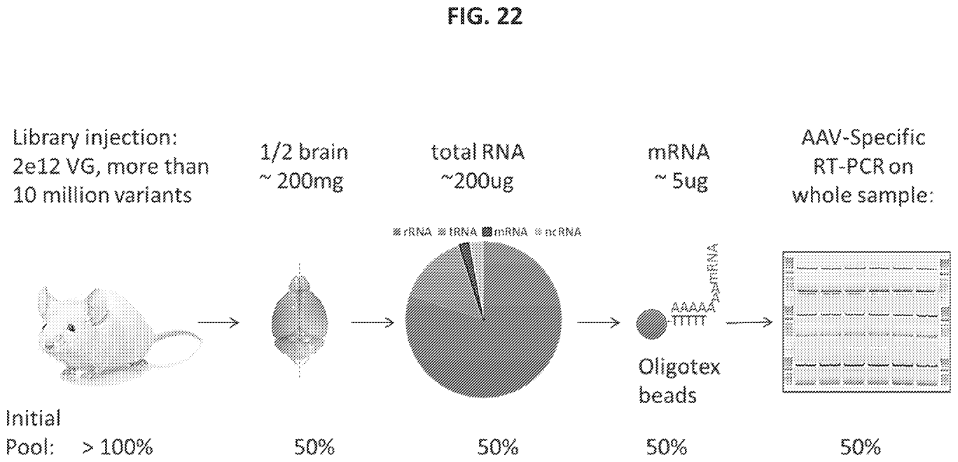

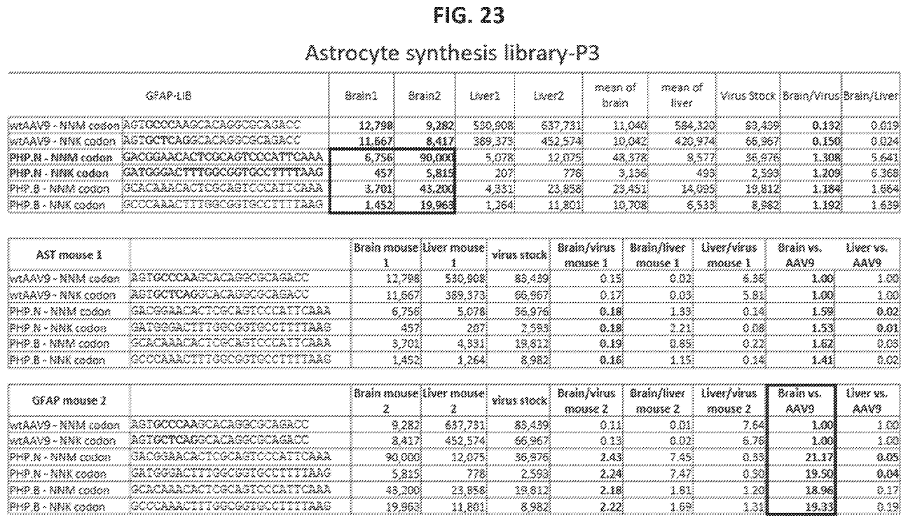

An RNA-driven library selection for increased brain transduction in a murine model was then developed. AAV9 libraries generated as described above were intravenously injected to male C57BL/6 mice at a dose of 2e12 VG per mouse. Two groups of mice were injected with a single SYN-driven or GFAP-driven libraries derived from wild-type AAV9 flanking sequences, and two other groups received pooled libraries containing wild-type and PHP.eB-derived flanking sequences (FIG. 21). After one month, RNA was extracted from 200 mg of brain tissue corresponding to a whole hemisphere using RNeasy Universal Plus procedure (Qiagen). In order to minimize the possibility of RNA under sampling, the entire RNA preparation (˜200 ug) was subjected to mRNA enrichment using Oligotex beads (Qiagen) as recommended by the manufacturer. The entire preparation of enriched mRNA (˜5 ug, equivalent to 2% of total RNA) was then reverse transcribed in a 40-ul Superscript IV reaction (Life Technologies) using a library-specific primer (SEQ ID NO: 67 (FIG. 22)). The entire pool of cDNA was then amplified 30 cycles with 55° C. annealing temperature and 2 minutes elongation in a 500-ul PCR reaction assembled with Q5 master mix, GloSpliceF6 forward primer and a CAP9-specific reverse primer (SEQ ID NO: 68). This method allowed recovery of abundant amplicons from all brain samples.

Full-length capsid amplicons were then used as templates for NGS library generation, as well as cloning into a P1 DNA library for the next round of biopanning, using the exact same assembly and cloning-free procedure. NGS analysis performed on PCR amplicons indicated that the library diversity dropped ˜25-fold (from 1e7 to 4e5) after the first round of biopanning for both Syn-driven and GFAP-driven libraries. The number of 1^{st} pass variants (P1) recovered was too high to show any significant sequence convergence at this point, and there was very little overlap between the composition of pools recovered from individual animals. Therefore, a second round of selection was performed. After the second biopanning (P2), the total number of unique variants further dropped by 4-5-fold, down to <1e5 peptides. Importantly, some libraries recovered after the first round of biopanning showed significant counts of wild-type AAV9 and AAV-PHP.eB sequences, presumably from environmental contamination. These later became useful benchmarks in the second round of enrichment.

Following RNA recovery and PCR amplification, a systematic enrichment analysis by NGS was performed by calculating the ratio of P2/P1 reads and comparing it to AAV9 or PHP.eB P2/P1 ratio. Several capsids showed a higher enrichment ratio than the benchmark PHP.eB in both Syn-driven and GFAP-driven libraries, and sequence convergence was observed.

Importantly, there was also a strong sequence convergence between different animals, suggesting an efficient selection after only two passages.

### Example 8. Multiplexing Selections

For the final multiplex in vivo screen by individual variant pooling in equimolar library, a subpopulation of variants with promising properties (such as, but not limited to, enrichment factor, liver detargeting, high counts in more than one mouse, etc.) may be selected and then an equimolar pool of primers encoding all the 7-mers (microchip solid-phase synthesis, up to 3,800 primers per chip) can be synthesized. The limited diversity library may be produced including internal controls such as, but not limited to, PHP.N, PHP.B, wild-type AAV9 (wtAAV9) and/or any other serotype including those taught herein. The mice are injected and then the RNA enrichment is compared to internal controls in a similar manner to a barcoding study, which is known in the art and described herein.

### Example 9. Codon Optimization

Codon variants may be used to improve data strength when using synthesized libraries. A listing of NNK codons, NNM codons and the most favorable NNM codons in mammals for various amino acids is provided in Table 3. In Table 3, * means that no NNM codon was available and ** means “avoid homopolymeric stretches if possible.”

TABLE 3
Codon Variants

| | | | Most |
|---|---|---|---|
| | | | favorable |
| Amino | | | NNM codon |
| acid | NNK codon | NNM codons | in mammals |
| F | TTT | TTC | TTC |
| L | TTG, CTT, CTG | TTA, CTC, CTA | CTC |
| S | TCT, TCG, AGT | TCC, TCA, AGC | AGC |
| Y | TAT | TAC | TAC |
| C | TGT | TGC | TGC |
| W | TGG | | TGG* |
| p | CCT, CCG | CCC, CCA | CCA** |
| H | CAT | CAC | CAC |
| Q | CAG | CAA | CAA |
| R | CGT, CGG, AGG | CGC, CGA, AGA | AGA |
| I | ATT | ATC, ATA | ATC |
| M | ATG | | ATT* |
| T | ACT, ACG | ACC, ACA | ACC |
| N | AAT | AAC | AAC |
| K | AAG | AAA | AAA |
| V | GTT, GTG | GTC, GTA | GTC |
| A | GCT, GCG | GCC, GCA | GCC |
| D | GAT | GAC | GAC |
| E | GAG | GAA | GAA |
| G | GGT, GGG | GGC, GGA | GGC |
| stop | TAG | TAC, TAA | n/a |

*no NNM codon available
**avoid homopolymetic stretches if possible

In order to have a balanced library it is recommended to establish a list of potential candidates. Then, using Table 3, a pooled primer library containing every peptide variant encoded by NNK codons (original from library) and non-NNK codons (maximum variation). If similar behavior is seen between the two variants of the same peptide, this would strengthen the analysis of that peptide. Additionally, it is recommended to choose the most favorable NNM codons (M=A or C).

### Example 10. Library Generation

The top-ranking peptide variants from SYN-driven and GFAP-driven libraries that showed enhanced performance relative to the parental AAV9 were selected. A de novo library by pooled primer synthesis of the peptide sequences plus AAV9, AAV-PHP.B and AAV-PHP.eB controls was generated. In order to exclude potential artifacts due to the DNA sequence and to increase the robustness of the assay, each peptide variant was encoded by two different DNA sequences, one where all amino acids were encoded by NNK codons (identical to the original library) and another one where NNM codons were used whenever possible (M=C or A).

Primer pools were produced by Twist biosciences using solid-phase synthesis and were used to generate a balanced library of nucleotide variants by PCR amplification of CAP C-terminus and Gibson assembly. Primers were provided at 1 fmole each, resulting in 0.6 pmole (regular PCR requires ˜25 pmole of primer). Primerless amplification on capsid gBlock template was performed over 10 cycles. Forward and reverse primers were added, followed by an additional 10, 15 or 20 PCR cycles. Constructs were then cloned into AAV9 backbone plasmids by Gibson/RCA (like regular libraries).

NGS analysis of SYN- and GFAP-driven AAV libraries produced with the pooled DNA showed a good correlation between the codon variants of each peptide, suggesting that the DNA sequence itself had little influence on virus production. The pooled synthetic library was injected intravenously to C57BL/6 mice (5e11 VG per mouse, N=9), BALB/C mice (5e11 VG per mouse, N=6) and to rats (5e12 VG per rat, N=6), and after one month in-life RNA was extracted from the brain and spinal cord, and DNA was extracted from liver and heart tissue samples for biodistribution analysis. Because the Synapsin and GFAP promoters are not fully active in non-CNS tissue, DNA was analyzed instead of RNA in peripheral organs. The initial focus was on the C57BL/6 mouse analysis because this is the mouse strain in which library evolution was performed.

The enrichment score of each capsid was determined by NGS analysis and defined as the ratio of reads per million (RPM) in the target tissue versus RPM in the inoculum. As expected from the published data, the PHP.B and PHP.eB (aka, PHP.N) capsids allowed significantly higher RNA expression in neurons compared to the AAV9 parental capsid (8-fold and 25-fold, respectively). There was a very high correlation between the codon variants of each peptide species in each animal (r=0.92, 0.93 and 0.95), confirming the robustness of the NGS assay.

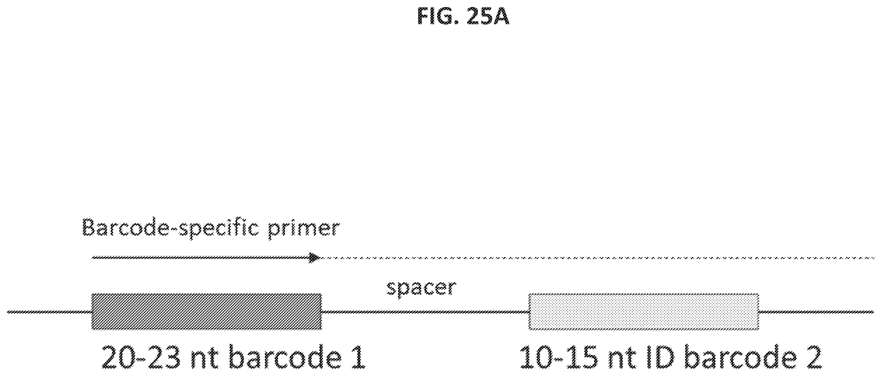

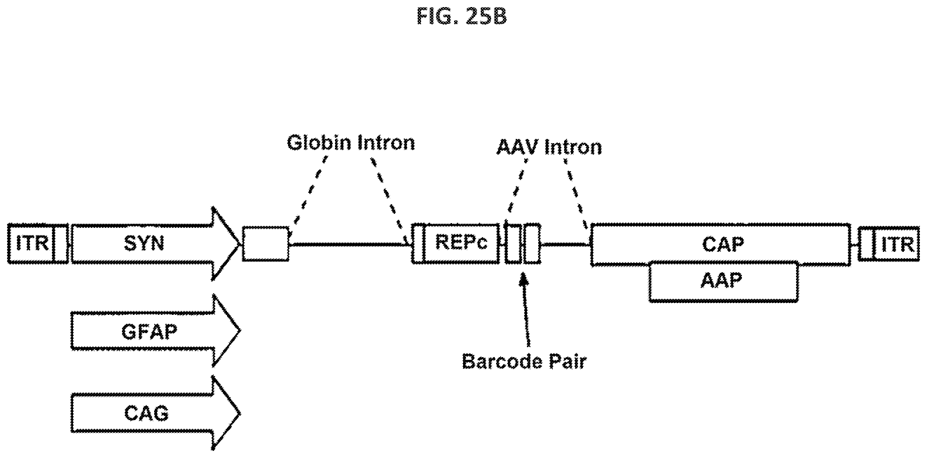

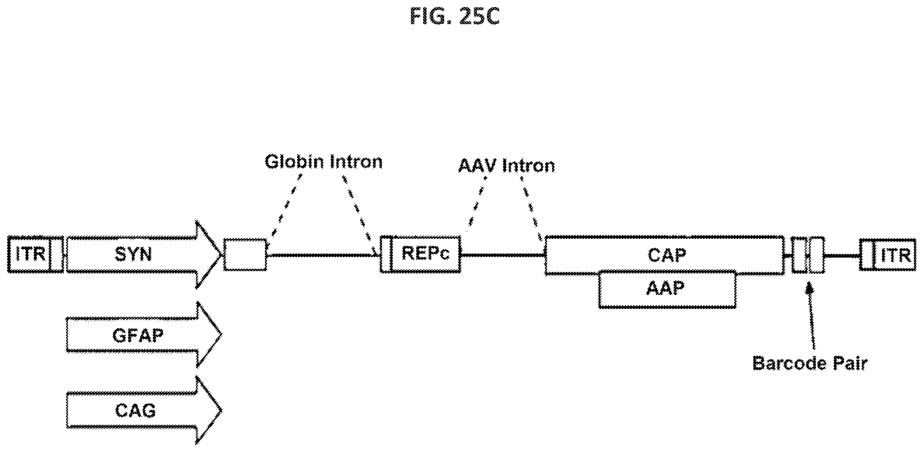

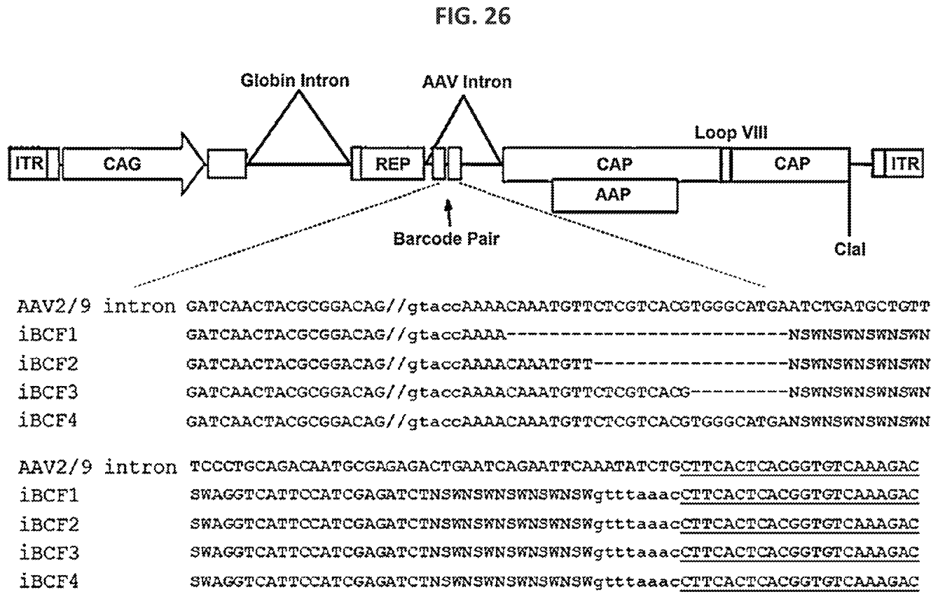

The capsid variants were ranked by average brain enrichment score from all animals A group of novel variants showed a higher enrichment score than the PHP.eB benchmark capsid in both neurons (Syn-driven) and astrocytes (GFAP-driven). Interestingly, many variants showed a different enrichment score in neurons vs. astrocytes, as indicated by the medium level of correlation between Syn- and GFAP-driven RNA. This suggests that certain capsids display an enhanced tropism for neurons, and others for astrocytes (FIG. 25).

### Example 11. Phylogenetic Grouping

Phylogenetic grouping of peptide sequences showed an evident correlation between sequence homology clusters and capsid phenotypes.

### Example 12. Capsid Testing

Capsid variants representative of distinct sequence clusters were chosen for individual transduction analysis in C57BL/6 mice. Each capsid was produced as a recombinant AAV packaging a self-complementary EGFP transgene driven by the ubiquitous promoter. Mouse groups (N=3) were injected intravenously with 6e10 VG and transduction efficiency was assessed after 1 month by quantifying EGFP mRNA in the brain, spinal cord, and liver tissue. EGFP mRNA expression was normalized using mouse TBP as a housekeeping gene, and DNA biodistribution was normalized to the single-copy mouse TfR gene. Reverse transcription was performed with the Quantitect kit and included a DNA removal treatment. All capsid variants showed a significant improvement in brain and spinal cord mRNA expression by comparison to the parent AAV9 capsid, and 3 out of 7 variants showed similar or higher transduction than the PHP.eB benchmark capsid. The viral DNA biodistribution showed a very strong tropism of several variants for the brain and spinal cord, but all the variants showed a 40- to 260-fold increase of biodistribution compared to AAV9.

Expected cellular tropism was tested using an NGS screen by labeling the neuronal NeuN marker. Within the cortex, the top capsids in the GFAP screen showed mostly GFP expression in NeuN-negative cells with glial morphology. Conversely, top capsids in the SYN screen showed a very high transduction of NeuN-positive cells, and the dual-specificity capsids—ranking high in both assays—showed mixed cell preference with multiple NeuN+ cells and glial cells.

Cellular tropism was also tested using mouse brain microvascular EC (mBMVEC) binding relative to AAV9.

Fluorescent EGFP expression in tissues of whole brain, cerebellum, cortex, and hippocampus revealed transduction patterns across a spectrum and demonstrated the identification of tissue-specific capsids.

The liver transduction, measured by mRNA expression and by whole tissue GFP expression, showed that several variants outperformed AAV9, which was unexpected in light of the NGS results. Some variants showed similar liver detargeting to AAV9.

### Example 13. Multi-Rodent Testing (Cross Species)

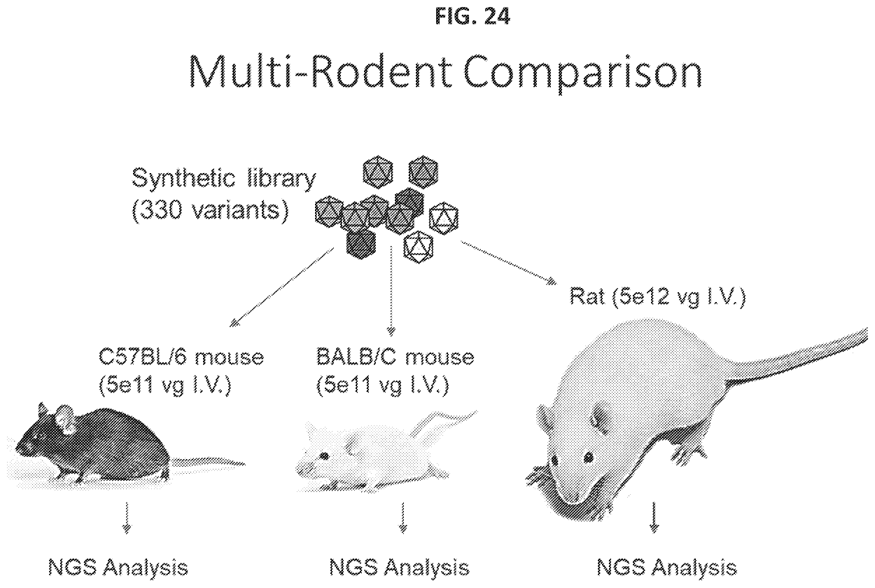

The efficacy of the capsid variants to transduce CNS was tested in other rodent strains or species (FIG. 24). Side-by-side comparison of neuron and astrocyte transduction in C57BL/6 mice, BALB/C mice and rats showed major differences in the enrichment scores of multiple variants between the two mouse strains, and even more pronounced differences between mice and rats. Strikingly, the most efficient capsid for rat brain transduction was the parental AAV9, which suggests that directed evolution “bottlenecks” capsid variants that are highly performant in one given species, as opposed to the versatility of wild-type AAV capsids.

Correlation analysis showed that some capsids shared high CNS transduction between C57BL/6 and BALB/C mice, whereas others were restricted to only one strain.

Interestingly, the PHP.B and PHP.eB capsid showed poor brain transduction in BALB/C mice, in line with a recent publication (Hordeaux et al., 2018, the contents of which are incorporated herein by reference in their entirety). When focusing on the capsids that showed >10-fold increase in brain transduction, 62 variants were improved only in C57BL/6 mice, 28 variants were improved only in BALB/C mice and 30 variants showed improved brain transduction in both strains. Consensus sequence analysis showed a “C57BL/6 signature” closely resembling the PHP.eB peptide whereas the BALB/C signature showed a different consensus, suggesting the use of a different cellular receptor.

The efficacy of the capsid variants to transduce CNS was also compared for C57BL/6 mice BMVEC and Human BMVEC.

### Example 14. Engineering of a NGS-Driven Selection System for Full-Length Capsid Variants

A barcode system was engineered to allow enrichment studies with full capsid length modifications. While the TRACER platform described here was initially developed for the use of peptide display libraries, where the randomized peptide sequence itself can be used for Illumina NGS analysis due to its short size, the Illumina sequencing technology does not typically allow sequencing of more than 300 contiguous bases, and therefore our platform cannot be used for NGS analysis of full-length capsid variants, such as those generated by DNA shuffling technology or error-prone PCR.

An alternative RNA-driven platform for full-length capsid libraries in which a unique molecular identifier (UMI) is placed outside the capsid gene and can be used for NGS enrichment analysis was designed (FIG. 25A-FIG. 25C). Once the variants with desired properties are identified by UMI enrichment analysis from animal tissue, the UMI sequence must allow highly specific recovery of the full-length capsid from the starting material with a minimal error rate. The system should have one or more of the following properties to be effective: 1) the UMI should be transcribed under control of a cell type-specific promoter, 2) the UMI should not interfere with capsid expression or splicing during virus production, 3) the UMI should be short enough for Illumina NGS sequencing (typically less than 60 nt for standard single-end 75 nt sequencing), and 4) the UMI should allow sequence-specific recovery of full-length capsids of interest from the starting DNA/virus library with a minimal error rate.

To address these properties: 1) the UMI was placed in the transcribed region of capsid library (i.e., anywhere between the transcription start site and the polyadenylation signal), 2) the UMI was placed either in various locations of the AAV intron (which mostly unspliced in the absence of helper functions) or between the capsid stop codon and the polyadenylation signal, 3) the UMI cassette was composed of two randomized 21-nt sequences separated by a 15-nt spacer, for a total length of 57 nt, which allows 18 extra nucleotides for primer annealing, and 4) the UMI randomized sequences were formed of NSW triplets (N=A, T, G, C; S=G, C; W=A, T) to prevent large variations in annealing temperature with amplification primers, avoid homopolymeric stretches and prevent the formation of a premature polyA signal (AATAAA).

Importantly, the UMI cassette contained two random sequences in tandem. The first sequence (outermost) is used to design a matching capsid recovery primer, and the second sequence (innermost) to confirm the identity of the capsid amplicon after cloning. This method should allow for the elimination of all clones containing non-specific amplification products. In an alternative embodiment, the innermost sequence can also be used to design a nested PCR primer in order to increase the specificity of amplification (FIG. 25A-FIG. 25C).

Several insertion sites of the tandem barcode to test the impact on virus viability and titers were explored. A series of constructs were engineered with the barcode inserted in the AAV intron of the CAG9 plasmid. Since AAV intron is spliced during virus production, the presence of the barcode should have only a minimal impact on the yields. Conversely, the AAV splicing is very ineffective in the absence of helper functions (Mouw et al., 2000, the contents of which are herein incorporated by reference in its entirety), therefore the barcode sequence will be preserved in the RNA recovered from animal tissue. All intronic barcode constructs were tested for their ability to produce high titer AAV progeny by co-transfecting them with pHelper and pREP3stop plasmids. All constructs allowed high titer AAV production going from 50% to 80% of non-barcoded CAG9 virus.

RNA splicing analysis from transfected cells showed that the rate of AAV intron splicing was slightly different between constructs and was more efficient when the intronic barcode was inserted after a conserved intervening sequence downstream of the splice donor.

Globin intron splicing was 100% effective in all tested conditions. As expected, AAV intron splicing was almost undetectable in the absence of helper functions.

An alternative platform was tested where the tandem barcode was placed between the capsid stop codon and the polyadenylation signal (FIG. 25C). Titers produced by the 3-barcoded constructs were identical to the non-barcoded CAG9 construct.

Overall, external barcoding of full-length capsid allows highly efficient AAV production, and the novel tandem barcode platform allows NGS-driven sequence-specific recovery from library preparations with high confidence.

### Example 15. Peptide Display Capsid Library Configuration

Peptide display capsid libraries are configured by insertion of randomized n-mer amino acids such as, but not limited to, 5-mer, 6-mer, 7-mer and/or 9-mer amino acids, into the surface-exposed hypervariable loop I, loop IV, loop VI, and/or loop VIII region of any AAV capsid serotype, including AAV5, AAV6, or AAV-DJ8, as well as AAV9 capsids, and/or variants thereof. The genes encoding the peptide display capsid library are under the control of any promotor, depending on the desired tropism, e.g., a neuron-specific synapsin promoter (SYN or Syn), or an astrocyte-specific GFAP promoter. Examples of such promoters are found in Table 2.

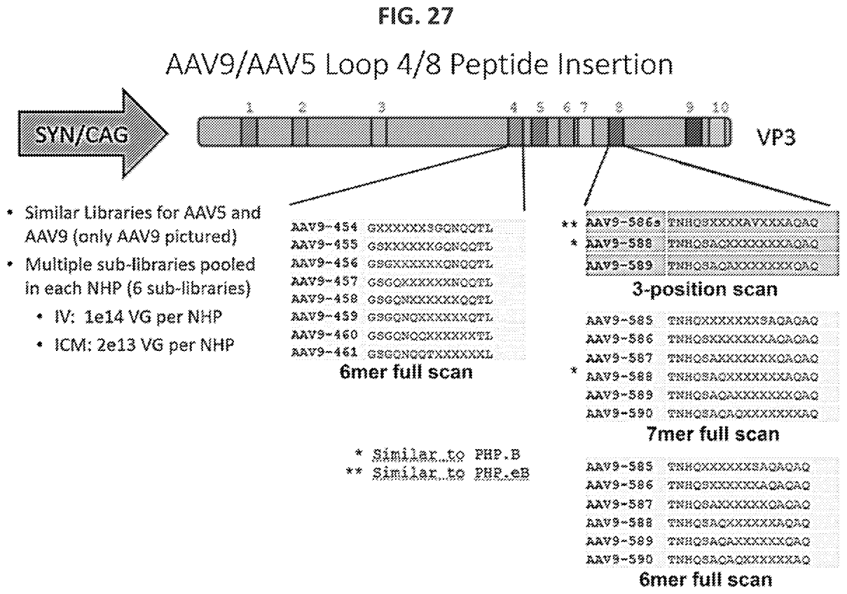

Peptide display capsid libraries are further configured such that the n-mer peptide insertion(s) follows a contiguous (or continuous) design and/or a noncontiguous (or noncontinuous), or split design, or combination thereof, with insertion position(s) mapped using a sliding window algorithm, as schematically represented in FIG. 27. As a non-limiting example, the peptide insertion may be an AAV9 6-mer contiguous peptide insertion with a sliding window originating at any amino acid position, e.g., amino acids 454-461. As another non-limiting example, the peptide insertion may be an AAV9 3-mer peptide split design or contiguous peptide insertion with a sliding window originating at any amino acid position, e.g., amino acids 586-588. As yet another non-limiting example, the peptide insertion may be an AAV9 6-mer and/or 7-mer peptide contiguous peptide insertion with a sliding window originating at any amino acid position, e.g., amino acids 585-590.

Any number of such configured peptide display capsid libraries may be pooled in a cell and/or subject, including a non-human primate (NHP) cell and/or subject, and administered to any tissue (e.g., central nervous system tissue) via any route, including but not limited to IV and/or ICM injection, at any VG/cell and/or VG/subject dose. As a non-limiting example, six configured peptide display capsid libraries are pooled and administered to the central nervous system of an NHP via intravenous administration of dose 1×10¹⁴ VG/NHP. As another non-limiting example, six libraries are pooled and administered to the central nervous system of NHP via an intraventricular administration, such as, but not limited to intraventricular administration that is an intra-cisterna magna injection (ICM) of dose 2×10¹³ VG/NHP.

### Example 16. Identification and Design of Non-Human Primate AAV Capsid Libraries

An RNA-driven library selection for increased nervous system tissue transduction in a non-human primate (NHP) is developed and carried out in accordance with methods similar, or equivalent, to those described in Example 7.

AAV libraries, e.g., AAV9 libraries, generated are administered by any route to NHPs at a given VG dose(s) per animal. A number of groups of NHPs are administered promoter-driven (e.g., SYN-driven or GFAP-driven) libraries derived from wild-type AAV9 flanking sequences, while other groups receive pooled libraries containing wild-type, PHP.eB-derived, or other AAV serotype flanking sequences. After a period, RNA is extracted from a tissue, such as but not limited to spinal cord and brain tissue. The RNA preparation is subjected to mRNA enrichment. The enriched mRNA is reverse transcribed to cDNA. The cDNA is amplified. This method allows recovery of abundant amplicons from tissue samples.

Full-length capsid amplicons are used as templates for NGS library generation, as well as cloning into DNA library for the next, or subsequent, round(s) of biopanning (FIG. 28A and FIG. 28B). Any number of rounds of AAV selection is performed. The total number of unique capsid variants may drop by a fold amount across AAV selection rounds. Capsid libraries may be pooled or combined at any step with any other capsid libraries described herein (FIG. 28B).

Following RNA recovery and PCR amplification, a systematic enrichment analysis by NGS is performed. Capsid enrichment ratio including comparison to a benchmark and sequence convergence is evaluated.

Peptide library candidates are evaluated and optimized using any of the methods described herein and are carried out, e.g., using methods similar, or equivalent, to those described in Example 8, Example 9, and/or Example 11. The top-ranking peptide variants are generated similar to as in Example 10, and transduction efficacy evaluated similar to as in Example 12 and Example 13.

### Example 17. Identification and Design of Orthogonal Evolution AAV Capsid Libraries

This study involves the use of orthogonal evolution wherein AAV particles may be administered for a first round of AAV selection across a set of any number of cell- and/or subject-types that may be from different species and/or strains; and, wherein any number of additional, i.e., subsequent, AAV selection rounds are performed either across a set of any number of cell- and/or subject-types that may be from different species and/or strains, or across a set of any number of cell- and/or subject-types that may be from the same species and/or strains, as represented in FIG. 29.

An RNA-driven library selection for increased nervous system tissue transduction to a set of any number of cell- and/or subject-types that may be from different species and/or strain is developed and carried out in accordance with methods similar, or equivalent, to those described in Example 7. AAV libraries, e.g., AAV9 libraries, generated are administered for a first round of AAV selection (biopanning) by any route to a non-human primate (NHP), a rodent (e.g., a rat), and/or a cell (e.g., a human brain microvascular endothelial cell, or hBMVEC) at a given VG dose(s) per subject and/or cell. A number of groups of NHPs, rodents, and/or cells are administered promoter-driven (e.g., SYN-driven or GFAP-driven) libraries derived from wild-type AAV9 flanking sequences, while other groups receive pooled libraries containing wild-type, PHP.eB-derived, or other AAV serotype flanking sequences. After a period, RNA is extracted from a tissue, such as but not limited to spinal cord and brain tissue. The RNA preparation is subjected to mRNA enrichment. The enriched mRNA is reverse transcribed to cDNA. The cDNA is amplified. This method allows recovery of abundant amplicons from tissue samples.

Full-length capsid amplicons are used as templates for NGS library generation, as well as cloning into DNA library for the next, or subsequent round(s) of biopanning. Subsequent rounds of biopanning are performed either across a set of any number of cell- and/or subject-types that may be from different species and/or strain as used in the above-described first round, or across a set of any number of cell- and/or subject-types that may be from the same species and/or strain as used in the above-described first round. Any number of rounds of selection is performed. The total number of unique capsid variants may drop by a fold amount across AAV selection rounds. Capsid libraries may be pooled or combined at any step with any other capsid libraries described herein (FIG. 29)

Following RNA recovery and PCR amplification, a systematic enrichment analysis by NGS is performed. Capsids enrichment ratio including comparison to a benchmark and sequence convergence is evaluated.

Peptide library candidates are evaluated and optimized using any of the methods described herein and are carried out, e.g., using methods similar, or equivalent, to those described in Example 8, Example 9, and/or Example 11. The top-ranking peptide variants are generated similar to as in Example 10, and transduction efficacy evaluated similar to as in Example 12 and Example 13.

We claim:

1. A method for generating a variant AAV capsid polypeptides, wherein relative to a parental AAV capsid polypeptide said variant AAV capsid polypeptides exhibit at least one of improved transduction or increased cell or tissue specificity, said method comprising:
a) generating a library of variant AAV capsid polypeptides, wherein said library comprises
i) a plurality of capsid polypeptides having a region of randomized sequence of 2, 3, 4, 5, 6, 7, 8, or 9 consecutive amino acids, or
ii) a plurality of capsid polypeptides from more than one parental AAV capsid polypeptide;
b) generating an AAV vector library by cloning the capsid polypeptides of libraries (i) or (ii) into AAV vectors, wherein said AAV vectors comprise a first promoter and a second promoter, wherein said second promoter drives capsid mRNA expression in the absence of helper virus co-infection.

2. A method of making a variant AAV capsid polypeptide, comprising:
(a) providing a plurality of vectors, e.g., a vector library, comprising nucleic acid molecules encoding:
(i) a plurality of variant AAV capsid polypeptides having a region of randomized sequence of at least 2, 3, 4, 5, 6, 7, 8, or 9 consecutive amino acids, or
(ii) a plurality of variant AAV capsid polypeptides from more than one parental AAV capsid polypeptide;
wherein the vectors comprise a first promoter and a second promoter, wherein the second promoter expresses capsid RNA, e.g., mRNA, in the absence of helper virus co-infection;
(b) generating a plurality of AAV particles comprising the plurality of vectors, e.g., the vector library, of (a); and
(c) administering the plurality of AAV particles to an NHP.

3. The method of claim 2, wherein the step (b) of generating plurality of AAV particles, comprises: (i) providing a host cell comprising the vector library; and (ii) incubating the host cell under conditions suitable to enclose the vectors in a capsid polypeptide, optionally wherein the host cell is an insect cell (e.g., a Sf9 cell) or a mammalian cell (e.g., a HEK293 cell).

4. The method of claim 2 or 3, wherein the plurality of vectors, e.g., vector library, comprises nucleic acid molecules encoding a plurality of variant AAV capsid polypeptides having a region of randomized sequence of at least 5, 6, 7, 8, or 9 consecutive amino acids.

5. The method of any one of the preceding claims, wherein the region of randomized sequence comprises a peptide insert of at least 4, 5, 6, 7, 8, or 9 consecutive amino acids.

6. The method of claim 5, wherein the insert is present in a surface-exposed hypervariable loop chosen from loop I, loop IV, loop VI, and/or loop VIII.

7. The method of claim 5 or 6, wherein the insert is present in loop IV and/or loop VIII of the parental AAV capsid polypeptide, optionally wherein the parental AAV capsid poly peptide comprises an AAV5 capsid polypeptide or an AAV9 capsid polypeptide.

8. The method of any one of claims 5-7, wherein:
(i) the insert is present immediately subsequent to a position selected from 454-461 of the parental sequence; and/or
(ii) the insert is present immediately subsequent to a position selected from 586-588 of the parental sequence.

9. The method of any one of claims 2-8, wherein the plurality of vectors, e.g., vector library, comprises nucleic acid molecules encoding a plurality of variant AAV capsid polypeptides from more than one parental AAV capsid polypeptide.

10. The method of any one the of preceding claims, wherein the parental AAV capsid polypeptide comprises an AAV5 capsid polypeptide or an AAV9 capsid polypeptide, e.g., an AAV9 capsid polypeptide of SEQ ID NO: 2.

11. The method of any one of claims 2-10, wherein the AAV particle or plurality of particles is administered via intravenous administration, intraventricular administration, or intra-cisternal magna (ICM) injection.

12. The method of any one of claims 2-11, wherein the method further comprises (d) collection and/or isolation of a target cell or tissue from the NHP, optionally wherein the target cell or tissue is collected and/or isolated at least about 5 to 21 days, e.g., about 5-10 days, 5-14 days, 7-10 days, 7-14 days, 7-21 days, 10-14 days, 10-21 days, 14-17 days, 5 days, 7 days, 10 days, 14 days, or 21 days, following administration of the AAV particles.

13. The method of am one of claims 2-12, wherein the method further comprises (e) recovery of RNA and/or antisense RNA encoding the variant AAV capsid polypeptides from the target cell or tissue, e.g., as described in Examples 15-17.

14. The method of claim 13, wherein the RNA encoding the variant AAV capsid polypeptides is enriched and/or reverse transcribed to cDNA, optionally wherein the cDNA is amplified, e.g., by PCR, e.g., as described in Examples 15-17.

15. The method of any one of claims 2-14, wherein the method further comprises (f) determination of the sequence of the variant AAV capsid polypeptides, e.g., by next generation sequence (NGS), e.g., as described in Examples 15-17.

16. The method of any one of claims 2-15, wherein the method further comprises (g) evaluating, e.g., measuring, the amount of the variant AAV capsid polypeptides (e.g., the amount of DNA encoding the variant AAV capsid polypeptides, the amount of RNA encoding the variant AAV capsid polypeptides, or the amount of the variant AAV capsid polypeptides), e.g., by NGS, e.g., as described in Examples 15-17, in a target cell, or tissue.

17. The method of claim 16, wherein the amount of the variant AAV capsid polypeptide in the target cell or tissue is increased relative to a reference level, wherein the reference level comprises the amount of a wild-type AAV capsid polypeptide (e.g., a wild-type AAV9 or AAV5 capsid polypeptide), or a parental capsid polypeptide (e.g., a parental capsid polypeptide comprising SEQ ID NO: 2) in the target cell or tissue.

18. The method of claim 16 or 17, wherein an increase in the amount of the variant AAV capsid polypeptide in the target cell or tissue is indicative of or predictive of:
(i) an increased level of transduction of the target cell or tissue, relative to a wild-type AAV capsid polypeptide (e.g., a wild-type AAV9 or AAV5 capsid polypeptide), or parental capsid polypeptide (e.g., a parental capsid polypeptide comprising SEQ ID NO: 2); and/or
(ii) increased tropism for the target cell or tissue, relative to a wild-type AAV capsid polypeptide (e.g., a wild-type AAV9 or AAV5 capsid polypeptide), or parental capsid polypeptide (e.g., a parental capsid polypeptide comprising SEQ ID NO: 2).

19. The method of any one of claims 2-18, which comprises repeating one, two, three, four, five, six, or all of steps (a)-(g), at least 1-5 times, e.g., at least 1-3 times, 2-3 times, 2-4 times, 3-5 times, 4-5 times, 1 time, 2 times, or 3 times.

20. The method of any one of claims 2-19, wherein responsive to an increase in one, two, or all of:
(i) the amount of the variant AAV capsid polypeptide in the target cell or tissue;
(ii) the level of transduction of the variant AAV capsid polypeptide in the target cell or tissue;
(iii) the tropism of the variant AAV capsid polypeptide for the target cell or tissue as compared to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2),
(f) selecting the variant AAV capsid polypeptide, e.g., for use in an AAV particle for delivering a payload to the target cell or tissue, e.g., of a subject, e.g. a human subject.

21. The method of any one of the preceding claims, wherein:
(i) the first and/or second promoter is located 5′ relative to a transgene encoding the variant AAV capsid polypeptide;
(ii) the first and/or second promoter is located 3′ relative to a transgene encoding the variant AAV capsid polypeptide;
(iii) the first promoter is located 5′ relative to a transgene encoding the variant AAV capsid polypeptide and the second promoter is located 3′ relative to the transgene encoding the variant AAV capsid polypeptide; or
(iv) the first promoter is located 3′ relative to a transgene encoding the variant AAV capsid polypeptide and the second promoter is located 5′ relative to the transgene encoding the variant AAV capsid polypeptide.

22. The method of any one of the preceding claims, wherein the first promoter is AAV2 P40.

23. The method of any one of the preceding claims, wherein the second promoter is:
(i) a ubiquitous promoter, a cell-type-specific promoter, or a tissue-specific promoter, or a functional variant thereof;
(ii) selected from any of those listed in Table 2, or a functional variant thereof;
(iii) a human elongation factor 1α-subunit (EF1α) promoter, cytomegalovirus (CMV) immediate-early enhancer and/or promoter, a chicken β-actin (CBA)) and/or its derivative CAG promoter, a β glucuronidase (GUSB) promoter; a ubiquitin C (UBC) promoter; or a functional variant thereof;
(iv) is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof; and/or
(v) a neuron specific promoter or an astrocyte-specific promoter, optionally wherein:
(i) the neuron specific promoter is a synapsin promoter, and/or
(ii) the astrocyte-specific promoter is a GFAP promoter.

24. The method of any one of the preceding claims, wherein:
(i) the first promoter is AAV2 P40 and the second promoter is a ubiquitous promoter, e.g., a human elongation factor 1α-subunit (EF1α) promoter, cytomegalovirus (CMV) immediate-early enhancer and/or promoter, a chicken β-actin (CBA)) and/or its derivative CAG promoter, a β glucuronidase (GUSB) promoter, a ubiquitin C (UBC) promoter, or a functional variant thereof; or
(ii) the first promoter is AAV2 P40 and the second promoter is a cell-type-specific promoter, e.g., a neuron-specific promoter or an astrocyte-specific promoter, or functional variant thereof.

25. The method of any one of claims 1-23, wherein:
(i) the first promoter is AAV2 P40 and the second promoter is a neuron-specific promoter;
(ii) the first promoter is AAV2 P40 and the second promoter is a synapsin promoter;
(iii) the first promoter is AAV2 P40 and the second promoter is an astrocyte-specific promoter; and/or
(iv) the first promoter is AAV2 P40 and the second promoter is a GFAP promoter.

26. The method of any one of the preceding claims, wherein the first and/or second promoters is operably linked to a transgene encoding the variant AAV capsid polypeptide.

27. The method of am one of claims 2-26, wherein the plurality of vectors further comprise an inverted terminal repeat (ITR) sequence, optionally wherein:
(i) the ITR sequence is positioned 5′ relative to the encoded variant AAV capsid polypeptide; and/or
(ii) the ITR sequence is positioned 3′ relative to the encoded variant AAV capsid polypeptide.

28. The method of any one of claims 2-27, wherein the plurality of vectors comprise an ITR sequence positioned 5′ relative to the encoded variant AAV capsid polypeptide, and an ITR sequence positioned 3′ relative to the encoded variant AAV capsid polypeptide.

29. The method of any one of 2-28, wherein the plurality of vectors further comprise a poly A signal sequence.

30. The method of any one of claims 2-29, wherein the plurality of vectors comprise in 5′ to 3′ order:
(i) a 5′ adeno-associated (AAV) ITR;
(ii) a ubiquitous promoter or a tissue specific promoter, optionally wherein:
(a) the ubiquitous promoter or the tissue specific promoter is selected from any of those listed in Table 2, or a functional variant thereof;
(b) the ubiquitous promoter is a human elongation factor 1α-subunit (EF1α) promoter; cytomegalovirus (CMV) immediate-early enhancer and/or promoter; a chicken β-actin (CBA)) and/or its derivative CAG promoter; a β glucuronidase (GUSB) promoter; a ubiquitin C (UBC) promoter; or a functional variant thereof;
(c) the tissue-specific promoter is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof;
(d) the tissue-specific promoter is a neuron-specific promoter, optionally a synapsin promoter; and/or
(e) the tissue-specific promoter is an astrocyte-specific promoter, optionally a GFAP promoter;
(iii) an AAV2 P40 promoter
(iv) a transgene encoding the variant AAV capsid polypeptide;
(v) a poly A signal sequence; and
(vi) a 3′ AAV ITR.

31. The method of any one of claims 2-29, wherein the vector or plurality of vectors comprise in 5′ to 3′ order:
(i) a 5′ adeno-associated (AAV) ITR;
(ii) an AAV2 P40 promoter;
(iii) a ubiquitous promoter or a tissue specific promoter, optionally wherein:
(a) the ubiquitous promoter or the tissue specific promoter is selected from any of those listed in Table 2, or a functional variant thereof;
(b) the ubiquitous promoter is a human elongation factor 1α-subunit (EF1α) promoter; cytomegalovirus (CMV) intermediate-early enhancer and/or promoter, a chicken β-actin (CBA)) and/or its derivative CAG promoter; a β glucuronidase (GUSB) promoter; a ubiquitin C (UBC) promoter; or a functional variant thereof;
(c) the tissue-specific promoter is a muscle specific promoter, a B cell promoter, a monocyte promoter, a leukocyte promoter, a macrophage promoter, a pancreatic acinar cell promoter, a endothelial cell promoter, a lung tissue promoter, an astrocyte-specific promoter, a nervous system-specific promoter, or functional variant thereof;
(d) the tissue-specific promoter is a neuron-specific promoter, optionally a synapsin promoter, and/or
(e) the tissue-specific promoter is an astrocyte-specific promoter, optionally a GFAP promoter;
(iv) a transgene encoding the variant AAV capsid polypeptide;
(v) a poly A signal sequence; and
(vi) a 3′ AAV ITR.

32. A variant AAV capsid polypeptide or plurality of variant AAV capsid polypeptides made by the method of any one of the preceding claims.

33. The variant AAV capsid polypeptide or plurality of variant AAV capsid polypeptides of claim 32, which:
(i) transduces a target cell or tissue at an increased level relative to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2); and/or
(ii) has increased tropism for a target cell or tissue relative to a reference level, e.g. a wild-type AAV capsid polypeptide (e.g., a wild-type AAV9 or AAV5 capsid polypeptide), or a parental capsid polypeptide (e.g., a parental capsid polypeptide comprising SEQ ID NO: 2).

34. The method of any one of claims 12-31, or the variant AAV capsid polypeptide or plurality of variant AAV capsid polypeptides of claim 32 or 33, wherein:
(i) the target cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell; and/or
(ii) the target tissue is:
(a) a CNS tissue, a PNS tissue, and/or a peripheral tissue; and/or
(b) a brain tissue (e.g., a cortex (e.g., frontal, parietal, occipital, temporal), thalamus, hypothalamus, striatum, putamen, caudate nucleus, hippocampus, entorhinal cortex, basal ganglia, deep cerebellar nuclei), a spinal cord tissue, a dorsal root ganglion, a muscle tissue, a liver tissue, a heart tissue, a gastrocnemius muscle tissue, a soleus muscle tissue, a pancreas tissue, a kidney tissue, a spleen tissue, a lung tissue, an adrenal glands tissue, a stomach tissue, a sciatic nerve tissue, a saphenous nerve tissue, a thyroid gland tissue, an eye tissue (with or without optic nerve), a pituitary gland tissue, a skeletal muscle tissue (rectus femoris), a colon tissue, a duodenum tissue, an ileum tissue, a jejunum tissue, a skin tissue of the leg, a superior cervical ganglia tissue, a urinary bladder tissue, an ovary tissue, a uterus tissue, a prostate gland tissue, and/or a testes tissue.

35. The method or variant AAV capsid polypeptide or plurality of variant AAV capsid polypeptides of any one of the preceding claims, wherein the variant AAV capsid polypeptide further comprises:
(i) an amino acid substitution at position K449, e.g., a K449R substitution, numbered according to SEQ ID NO: 2;
(i) the amino acid sequence of SEQ ID NO: 2, or an amino acid sequence at least 90% (e.g., at least 92, 95, 96, 97, 98, or 99%) identical thereto;
(ii) an amino acid sequence encoded by the nucleotide sequence of SEQ ID NO: 1, or a nucleotide at least 90% (e.g., at least 92, 95, 96, 97, 98, or 99%) identical thereto; and/or
(iii) the amino acid sequence of SEQ ID NO: 3, or an amino acid sequence at least 90% (e.g., at least 92, 95, 96, 97, 98, or 99%) identical thereto, optionally provided that position 449 is not K, e.g., is R.

36. A library comprising a plurality of variant AAV capsid polypeptides generated according to the method of any one of claims 1-31, or 34-35.

37. A cell comprising the variant AAV capsid polypeptide or plurality of variant AAV capsid polypeptides of any one of claims 32-35, or the library of variant AAV capsid polypeptides of claim 36, optionally wherein:
(i) the cell is an insect cell (e.g., an Sf9 cell), prokaryotic cell, or a eukaryotic cell (e.g., a mammalian cell, a human cell, an NHP cell, an HEK293 cell, an hBMVEC, and/or an NHP BMVEC); and/or
(ii) the cell is a neuronal cell, a neural stem cell, an astrocyte, an oligodendrocyte, a microglia cell, a retinal cell, a tumor cell, a hematopoietic stem cell, an insulin producing beta cell, a lung epithelium cell, an endothelial cell, a liver cell, a skeletal muscle cell, a muscle stem cell, a muscle satellite cell, or a cardiac muscle cell.


SEQUENCE LISTING (89 sequences)

SEQ ID NO: 1 - DNA, 2211 bp, Unknown
  source: /note=“Description of Unknown adeno-associated virus, human clone 9”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 2 - protein, 736 aa, Unknown
  source: /note=“Description of Unknown capsid of hu.14/AAV9”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  source: /note=“Description of Artificial Sequence Synthetic AAV9 Capsid Sequence”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 2470 bp, Artificial Sequence
  source: /note=“Description of Artificial Sequence Synthetic polynucleotide”
cgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggtccaccataccttcgattatccgatttgcttgttaatcaataaaccgtttaattcgtttcagttgaactttggtctctgcgtatttctttcttatctagtttccatgctctagagcggccgccaccgcggtggagctccagcttttgt

SEQ ID NO: 5 - DNA, 3681 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatcgtttaaaccgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgggagcggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - DNA, 3755 bp, Artificial Sequence
  source: /note=“Description of Artificial Sequence Synthetic polynucleotide”
gtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctc

SEQ ID NO: 7 - DNA, 3755 bp, Artificial Sequence
  source: /note=“Description of Artificial Sequence Synthetic polynucleotide”
gtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttagcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagtagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataactaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctc

SEQ ID NO: 8 - DNA, 3710 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatctagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 9 - DNA, 3852 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatcgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 4425 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 11 - DNA, 4480 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 12 - DNA, 4338 bp, Artificial Sequence
  source: /note=“Description of Artificial Sequence Synthetic polynucleotide”
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgttagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 20 bp, Artificial Sequence
  source: /note=“Description of Artificial Sequence Synthetic oligonucleotide”
gtgccaagagtgacctcctg

SEQ ID NO: 14 - DNA, 444 bp, Artificial Sequence
  source: /note=“Description of Artificial Sequence Synthetic polynucleotide”
actgcccccgcgaccggcacgtacaacctccaggaaatcgtgcccggcagcgtgtggatggagagggacgtgtacctccaaggacccatctgggccaagatcccagagacgggggcgcactttcacccctctccggctatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 15 - DNA, 440 bp, Artificial Sequence
  source: /note=“Description of Artificial Sequence Synthetic polynucleotide”
accggagatgtgcatgttatgggagccttacctggaatggtgtggcaagacagggacgtctacctgcagggtcctatttgggccaaaattcctcacacggatggacactttcacccatctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcaatatacatctaactatgcaaaatctgccaacgttgatttcactgtggacaacaatggactttatactgagcctcgccccattggcacccgttacctcacccgtcccctgtaatcgat

SEQ ID NO: 16 - DNA, 447 bp, Artificial Sequence
  source: /note=“Description of Artificial Sequence Synthetic polynucleotide”
acacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggaccctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 17 - DNA, 447 bp, Artificial Sequence
  source: /note=“Description of Artificial Sequence Synthetic polynucleotide”
gcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 18 - DNA, 4314 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgttagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 19 - DNA, 4456 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 20 - DNA, 4283 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgttagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcacaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctctgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 21 - DNA, 4426 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctctgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 22 - DNA, 4313 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgttagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcacaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 23 - DNA, 4456 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 24 - DNA, 4319 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgttagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcacaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcaaggtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 25 - DNA, 4462 bp, Artificial Sequence
  source: /note=“Description of Artificial Sequence Synthetic polynucleotide”
tatcagcacacaatagtccattatacgcgcgtataatgggcaattgtgtgctgatacagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctagcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgatatccatgcgtcgacataacgcgtgatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtcgccaccggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcaaggtgtacatcgattgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagcatgcaattaactggccgtcgttttacaacgtcgtgactgggaaaaccctggcgttacccaacttaatcgccttgcagcacatccccctttcgccagctgtatcagcacacaattgcccattatacgcgcgtataatggactattgtgtgctgata

SEQ ID NO: 26 - DNA, 20 bp, Artificial Sequence
  source: /note=“Description of Artificial Sequence Synthetic oligonucleotide”
ccgtgccaagagtgacctcc

SEQ ID NO: 27 - DNA, 51 bp, Artificial Sequence
  source: /note=“Description of Artificial Sequence Synthetic oligonucleotide”
  CDS (1)..(51)
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
ctccagagtagcagcnnknnknnknnknnknnknnkacagaccctgcgacc

SEQ ID NO: 28 - protein, 17 aa, Artificial Sequence
  source: /note=“Description of Artificial Sequence Synthetic peptide”
  MOD_RES (6)..(12): Any naturally occurring amino acid
LQSSSXXXXXXXTDPAT

SEQ ID NO: 29 - DNA, 51 bp, Artificial Sequence
  source: /note=“Description of Artificial Sequence Synthetic oligonucleotide”
  CDS (1)..(51)
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
aaccagagctctaccnnknnknnknnknnknnknnkactgcccccgcgacc

SEQ ID NO: 30 - protein, 17 aa, Artificial Sequence
  source: /note=“Description of Artificial Sequence Synthetic peptide”
  MOD_RES (6)..(12): Any naturally occurring amino acid
NQSSTXXXXXXXTAPAT

SEQ ID NO: 31 - DNA, 51 bp, Artificial Sequence
  source: /note=“Description of Artificial Sequence Synthetic oligonucleotide”
  CDS (1)..(51)
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
ctccagcaaggtaacnnknnknnknnknnknnknnkacacaagcagctacc

SEQ ID NO: 32 - protein, 17 aa, Artificial Sequence
  source: /note=“Description of Artificial Sequence Synthetic peptide”
  MOD_RES (6)..(12): Any naturally occurring amino acid
LQQGNXXXXXXXTQAAT

SEQ ID NO: 33 - DNA, 30 bp, Artificial Sequence
  source: /note=“Description of Artificial Sequence Synthetic oligonucleotide”
  CDS (1)..(30)
caccagagtgcccaagcacaggcgcagacc

SEQ ID NO: 34 - protein, 10 aa, Artificial Sequence
  source: /note=“Description of Artificial Sequence Synthetic peptide”
HQSAQAQAQT

SEQ ID NO: 35 - DNA, 51 bp, Artificial Sequence
  source: /note=“Description of Artificial Sequence Synthetic oligonucleotide”
  CDS (1)..(51)
caccagagtgatgggactttggcggtgccttttaaggcacaggcgcagacc

SEQ ID NO: 36 - protein, 17 aa, Artificial Sequence
  source: /note=“Description of Artificial Sequence Synthetic peptide”
HQSDGTLAVPFKAQAQT

SEQ ID NO: 37 - DNA, 51 bp, Artificial Sequence
  source: /note=“Description of Artificial Sequence Synthetic oligonucleotide”
  CDS (1)..(51)
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
caccagagtgcccaannknnknnknnknnknnknnkgcacaggcgcagacc

SEQ ID NO: 38 - protein, 17 aa, Artificial Sequence
  source: /note=“Description of Artificial Sequence Synthetic peptide”
  MOD_RES (6)..(12): Any naturally occurring amino acid
HQSAQXXXXXXXAQAQT

SEQ ID NO: 39 - DNA, 51 bp, Artificial Sequence
  source: /note=“Description of Artificial Sequence Synthetic oligonucleotide”
  CDS (1)..(51)
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
caccagagtgatgggnnknnknnknnknnknnknnkgcacaggcgcagacc

SEQ ID NO: 40 - protein, 17 aa, Artificial Sequence
  source: /note=“Description of Artificial Sequence Synthetic peptide”
  MOD_RES (6)..(12): Any naturally occurring amino acid
HQSDGXXXXXXXAQAQT

SEQ ID NO: 41 - DNA, 51 bp, Artificial Sequence
  source: /note=“Description of Artificial Sequence Synthetic oligonucleotide”
  CDS (1)..(51)
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
caccagagtgatggcaccnnknnknnknnknnknnkgcacaggcgcagacc

SEQ ID NO: 42 - protein, 17 aa, Artificial Sequence
  source: /note=“Description of Artificial Sequence Synthetic peptide”
  MOD_RES (7)..(12): Any naturally occurring amino acid
HQSDGTXXXXXXAQAQT

SEQ ID NO: 43 - DNA, 45 bp, Artificial Sequence
  source: /note=“Description of Artificial Sequence Synthetic oligonucleotide”
gccaccaacaaccagagctctgtacatcgattgttaatcaataaa

SEQ ID NO: 44 - DNA, 45 bp, Artificial Sequence
  source: /note=“Description of Artificial Sequence Synthetic oligonucleotide”
gtggcagtcaatctccagagtgtacatcgattgttaatcaataaa

SEQ ID NO: 45 - DNA, 45 bp, Artificial Sequence
  source: /note=“Description of Artificial Sequence Synthetic oligonucleotide”
tctaccaacctccagcaaggtgtacatcgattgttaatcaataaa

SEQ ID NO: 46 - DNA, 45 bp, Artificial Sequence
  source: /note=“Description of Artificial Sequence Synthetic oligonucleotide”
gtggccacaaaccaccagagtgtacatcgattgttaatcaataaa

SEQ ID NO: 47 - DNA, 21 bp, Artificial Sequence
  source: /note=“Description of Artificial Sequence Synthetic oligonucleotide”
  modified_base (1)..(2): a, c, t, g, unknown or other
  modified_base (4)..(5): a, c, t, g, unknown or other
  modified_base (7)..(8): a, c, t, g, unknown or other
  modified_base (10)..(11): a, c, t, g, unknown or other
  modified_base (13)..(14): a, c, t, g, unknown or other
  modified_base (16)..(17): a, c, t, g, unknown or other
  modified_base (19)..(20): a, c, t, g, unknown or other
nnknnknnknnknnknnknnk

SEQ ID NO: 48 - DNA, 71 bp, Artificial Sequence
  source: /note=“Description of Artificial Sequence Synthetic oligonucleotide”
  CDS (1)..(69)
  modified_base (31)..(32): a, c, t, g, unknown or other
  modified_base (34)..(35): a, c, t, g, unknown or other
  modified_base (37)..(38): a, c, t, g, unknown or other
  modified_base (40)..(41): a, c, t, g, unknown or other
  modified_base (43)..(44): a, c, t, g, unknown or other
  modified_base (46)..(47): a, c, t, g, unknown or other
  modified_base (49)..(50): a, c, t, g, unknown or other
caagtggccacaaaccaccagagtgcccaannknnknnknnknnknnknnkgcacaggcgcagaccggctg

SEQ ID NO: 49 - protein, 23 aa, Artificial Sequence
  source: /note=“Description of Artificial Sequence Synthetic peptide”
  MOD_RES (11)..(17): Any naturally occurring amino acid
QVATNHQSAQXXXXXXXAQAQTG

SEQ ID NO: 50 - DNA, 69 bp, Artificial Sequence
  source: /note=“Description of Artificial Sequence Synthetic oligonucleotide”
  CDS (1)..(69)
  modified_base (31)..(32): a, c, t, g, unknown or other
  modified_base (34)..(35): a, c, t, g, unknown or other
  modified_base (37)..(38): a, c, t, g, unknown or other
  modified_base (40)..(41): a, c, t, g, unknown or other
  modified_base (43)..(44): a, c, t, g, unknown or other
  modified_base (46)..(47): a, c, t, g, unknown or other
  modified_base (49)..(50): a, c, t, g, unknown or other
caggtcgctaccaatcatcaatccgcacagnnknnknnknnknnknnknnkgctcaagcacaaacagga

SEQ ID NO: 51 - DNA, 24 bp, Artificial Sequence
  source: /note=“Description of Artificial Sequence Synthetic oligonucleotide”
caagtggccacaaaccaccagagt

SEQ ID NO: 52 - DNA, 21 bp, Artificial Sequence
  source: /note=“Description of Artificial Sequence Synthetic oligonucleotide”
caggtcgctaccaatcatcaa

SEQ ID NO: 53 - DNA, 24 bp, Artificial Sequence
  source: /note=“Description of Artificial Sequence Synthetic oligonucleotide”
agtgcccaagcacaggcgcagacc

SEQ ID NO: 54 - DNA, 24 bp, Artificial Sequence
  source: /note=“Description of Artificial Sequence Synthetic oligonucleotide”
agtgctcaggcacaggcgcagacc

SEQ ID NO: 55 - DNA, 27 bp, Artificial Sequence
  source: /note=“Description of Artificial Sequence Synthetic oligonucleotide”
gacggaacactcgcagtcccattcaaa

SEQ ID NO: 56 - DNA, 27 bp, Artificial Sequence
  source: /note=“Description of Artificial Sequence Synthetic oligonucleotide”
gatgggactttggcggtgccttttaag

SEQ ID NO: 57 - DNA, 27 bp, Artificial Sequence
  source: /note=“Description of Artificial Sequence Synthetic oligonucleotide”
gcacaaacactcgcagtcccattcaaa

SEQ ID NO: 58 - DNA, 27 bp, Artificial Sequence
  source: /note=“Description of Artificial Sequence Synthetic oligonucleotide”
gcccaaactttggcggtgccttttaag

SEQ ID NO: 59 - DNA, 56 bp, Unknown
  source: /note=“Description of Unknown protelomerase recognition sequence”
tatcagcacacaattgcccattatacgcgcgtataatggactattgtgtgctgata

SEQ ID NO: 60 - DNA, 56 bp, Unknown
  source: /note=“Description of Unknown protelomerase sequence”
tatcagcacacaattgcccattatacgcgcgtataatgggcaattgtgtgctgata

SEQ ID NO: 61 - DNA, 56 bp, Unknown
  source: /note=“Description of Unknown protelomerase sequence”
tatcagcacacaatagtccattatacgcgcgtataatggactattgtgtgctgata

SEQ ID NO: 62 - DNA, 133 bp, Artificial Sequence
  source: /note=“Description of Artificial Sequence Synthetic polynucleotide”
gatcaactacgcggacaggtaccaaaacaaatgttctcgtcacgtgggcatgaatctgatgctgtttccctgcagacaatgcgagagactgaatcagaattcaaatatctgcttcactcacggtgtcaaagac

SEQ ID NO: 63 - DNA, 107 bp, Artificial Sequence
  source: /note=“Description of Artificial Sequence Synthetic polynucleotide”
  modified_base (28)..(28): a, c, t, g, unknown or other
  modified_base (31)..(31): a, c, t, g, unknown or other
  modified_base (34)..(34): a, c, t, g, unknown or other
  modified_base (37)..(37): a, c, t, g, unknown or other
  modified_base (40)..(40): a, c, t, g, unknown or other
  modified_base (63)..(63): a, c, t, g, unknown or other
  modified_base (66)..(66): a, c, t, g, unknown or other
  modified_base (69)..(69): a, c, t, g, unknown or other
  modified_base (72)..(72): a, c, t, g, unknown or other
  modified_base (75)..(75): a, c, t, g, unknown or other
gatcaactacgcggacaggtaccaaaanswnswnswnswnswaggtcattccatcgagatctnswnswnswnswnswgtttaaaccttcactcacggtgtcaaagac

SEQ ID NO: 64 - DNA, 115 bp, Artificial Sequence
  source: /note=“Description of Artificial Sequence Synthetic polynucleotide”
  modified_base (36)..(36): a, c, t, g, unknown or other
  modified_base (39)..(39): a, c, t, g, unknown or other
  modified_base (42)..(42): a, c, t, g, unknown or other
  modified_base (45)..(45): a, c, t, g, unknown or other
  modified_base (48)..(48): a, c, t, g, unknown or other
  modified_base (71)..(71): a, c, t, g, unknown or other
  modified_base (74)..(74): a, c, t, g, unknown or other
  modified_base (77)..(77): a, c, t, g, unknown or other
  modified_base (80)..(80): a, c, t, g, unknown or other
  modified_base (83)..(83): a, c, t, g, unknown or other
gatcaactacgcggacaggtaccaaaacaaatgttnswnswnswnswnswaggtcattccatcgagatctnswnswnswnswnswgtttaaaccttcactcacggtgtcaaagac

SEQ ID NO: 65 - DNA, 124 bp, Artificial Sequence
  source: /note=“Description of Artificial Sequence Synthetic polynucleotide”
  modified_base (45)..(45): a, c, t, g, unknown or other
  modified_base (48)..(48): a, c, t, g, unknown or other
  modified_base (51)..(51): a, c, t, g, unknown or other
  modified_base (54)..(54): a, c, t, g, unknown or other
  modified_base (57)..(57): a, c, t, g, unknown or other
  modified_base (80)..(80): a, c, t, g, unknown or other
  modified_base (83)..(83): a, c, t, g, unknown or other
  modified_base (86)..(86): a, c, t, g, unknown or other
  modified_base (89)..(89): a, c, t, g, unknown or other
  modified_base (92)..(92): a, c, t, g, unknown or other
gatcaactacgcggacaggtaccaaaacaaatgttctcgtcacgnswnswnswnswnswaggtcattccatcgagatctnswnswnswnswnswgtttaaaccttcactcacggtgtcaaagac

SEQ ID NO: 66 - DNA, 133 bp, Artificial Sequence
  source: /note=“Description of Artificial Sequence Synthetic polynucleotide”
  modified_base (54)..(54): a, c, t, g, unknown or other
  modified_base (57)..(57): a, c, t, g, unknown or other
  modified_base (60)..(60): a, c, t, g, unknown or other
  modified_base (63)..(63): a, c, t, g, unknown or other
  modified_base (66)..(66): a, c, t, g, unknown or other
  modified_base (89)..(89): a, c, t, g, unknown or other
  modified_base (92)..(92): a, c, t, g, unknown or other
  modified_base (95)..(95): a, c, t, g, unknown or other
  modified_base (98)..(98): a, c, t, g, unknown or other
  modified_base (101)..(101): a, c, t, g, unknown or other
gatcaactacgcggacaggtaccaaaacaaatgttctcgtcacgtgggcatganswnswnswnswnswaggtcattccatcgagatctnswnswnswnswnswgtttaaaccttcactcacggtgtcaaagac

SEQ ID NO: 67 - DNA, 38 bp, Artificial Sequence
  source: /note=“Description of Artificial Sequence Synthetic primer”
gaaacgaattaaacggtttattgattaacaatcgatta

SEQ ID NO: 68 - DNA, 45 bp, Artificial Sequence
  source: /note=“Description of Artificial Sequence Synthetic primer”
cggtttattgattaacaatcgattacagattacgagtcaggtatc

SEQ ID NO: 69 - protein, 15 aa, Artificial Sequence
  source: /note=“Description of Artificial Sequence Synthetic peptide”
  MOD_RES (2)..(7): Any amino acid
GXXXXXXSGQNQQTL

SEQ ID NO: 70 - protein, 15 aa, Artificial Sequence
  source: /note=“Description of Artificial Sequence Synthetic peptide”
  MOD_RES (3)..(8): Any amino acid
GSXXXXXXGQNQQTL

SEQ ID NO: 71 - protein, 15 aa, Artificial Sequence
  source: /note=“Description of Artificial Sequence Synthetic peptide”
  MOD_RES (4)..(9): Any amino acid
GSGXXXXXXQNQQTL

SEQ ID NO: 72 - protein, 15 aa, Artificial Sequence
  source: /note=“Description of Artificial Sequence Synthetic peptide”
  MOD_RES (5)..(10): Any amino acid
GSGQXXXXXXNQQTL

SEQ ID NO: 73 - protein, 15 aa, Artificial Sequence
  source: /note=“Description of Artificial Sequence Synthetic peptide”
  MOD_RES (6)..(11): Any amino acid
GSGQNXXXXXXQQTL

SEQ ID NO: 74 - protein, 15 aa, Artificial Sequence
  source: /note=“Description of Artificial Sequence Synthetic peptide”
  MOD_RES (7)..(12): Any amino acid
GSGQNQXXXXXXQTL

SEQ ID NO: 75 - protein, 15 aa, Artificial Sequence
  source: /note=“Description of Artificial Sequence Synthetic peptide”
  MOD_RES (8)..(13): Any amino acid
GSGQNQQXXXXXXTL

SEQ ID NO: 76 - protein, 15 aa, Artificial Sequence
  source: /note=“Description of Artificial Sequence Synthetic peptide”
  MOD_RES (9)..(14): Any amino acid
GSGQNQQTXXXXXXL

SEQ ID NO: 77 - protein, 18 aa, Artificial Sequence
  source: /note=“Description of Artificial Sequence Synthetic peptide”
  MOD_RES (6)..(9): Any amino acid
  MOD_RES (12)..(14): Any amino acid
TNHQSXXXXAVXXXAQAQ

SEQ ID NO: 78 - protein, 18 aa, Artificial Sequence
  source: /note=“Description of Artificial Sequence Synthetic peptide”
  MOD_RES (8)..(14): Any amino acid
TNHQSAQXXXXXXXAQAQ

SEQ ID NO: 79 - protein, 18 aa, Artificial Sequence
  source: /note=“Description of Artificial Sequence Synthetic peptide”
  MOD_RES (9)..(15): Any amino acid
TNHQSAQAXXXXXXXQAQ

SEQ ID NO: 80 - protein, 18 aa, Artificial Sequence
  source: /note=“Description of Artificial Sequence Synthetic peptide”
  MOD_RES (5)..(11): Any amino acid
TNHQXXXXXXXSAQAQAQ

SEQ ID NO: 81 - protein, 18 aa, Artificial Sequence
  source: /note=“Description of Artificial Sequence Synthetic peptide”
  MOD_RES (6)..(12): Any amino acid
TNHQSXXXXXXXAQAQAQ

SEQ ID NO: 82 - protein, 18 aa, Artificial Sequence
  source: /note=“Description of Artificial Sequence Synthetic peptide”
  MOD_RES (7)..(13): Any amino acid
TNHQSAXXXXXXXQAQAQ

SEQ ID NO: 83 - protein, 18 aa, Artificial Sequence
  source: /note=“Description of Artificial Sequence Synthetic peptide”
  MOD_RES (10)..(16): Any amino acid
TNHQSAQAQXXXXXXXAQ

SEQ ID NO: 84 - protein, 17 aa, Artificial Sequence
  source: /note=“Description of Artificial Sequence Synthetic peptide”
  MOD_RES (5)..(10): Any amino acid
TNHQXXXXXXSAQAQAQ

SEQ ID NO: 85 - protein, 17 aa, Artificial Sequence
  source: /note=“Description of Artificial Sequence Synthetic peptide”
  MOD_RES (6)..(11): Any amino acid
TNHQSXXXXXXAQAQAQ

SEQ ID NO: 86 - protein, 17 aa, Artificial Sequence
  source: /note=“Description of Artificial Sequence Synthetic peptide”
  MOD_RES (7)..(12): Any amino acid
TNHQSAXXXXXXQAQAQ

SEQ ID NO: 87 - protein, 17 aa, Artificial Sequence
  source: /note=“Description of Artificial Sequence Synthetic peptide”
  MOD_RES (8)..(13): Any amino acid
TNHQSAQXXXXXXAQAQ

SEQ ID NO: 88 - protein, 17 aa, Artificial Sequence
  source: /note=“Description of Artificial Sequence Synthetic peptide”
  MOD_RES (9)..(14): Any amino acid
TNHQSAQAXXXXXXQAQ

SEQ ID NO: 89 - protein, 17 aa, Artificial Sequence
  source: /note=“Description of Artificial Sequence Synthetic peptide”
  MOD_RES (10)..(15): Any amino acid
TNHQSAQAQXXXXXXAQ